(19) United States
(12) Patent Application Publication
(10) Pub. No.: US 2024/0158390 A1
(43) Pub. Date: May 16, 2024

(54) OTHER HETEROAROMATIC COMPOUNDS HAVING ACTIVITY AGAINST RSV

(71) Applicant: Janssen Sciences Ireland Unlimited Company, Co. Cork (IE)

(72) Inventors: Guillaume Jean Maurice MERCEY, Montaure (FR); David Francis Alain LANCOIS, Louviers (FR); Antoine Benjamin MICHAUT, LeVaudreuil (FR); Tony Félicien BOUISSET, Louviers (FR); Jérôme Émile Georges GUILLEMONT, Andé (FR); Pierre Jean-Marie Bernard RABOISSON, Wavre (FR)

(21) Appl. No.: 17/595,468

(22) PCT Filed: May 20, 2020
(86) PCT No.: PCT/EP2020/064030

§ 371 Date: Nov. 17, 2021

(30) Foreign Application Priority Data
May 23, 2019 (EP) 19176264.0

(51) Int. Cl. C07D 471/04; A61K 45/06; A61P 31/14; C07D 401/06; C07D 401/14; C07D 405/14; C07D 413/14; C07D 417/14; C07D 491/048; C07D 495/04; C07D 498/04; C07D 513/04

(52) U.S. Cl. CPC C07D 471/04; A61K 45/06; A61P 31/14; C07D 401/06; C07D 401/14; C07D 405/14; C07D 413/14; C07D 417/14; C07D 491/048; C07D 495/04; C07D 498/04; C07D 513/04

(57) ABSTRACT

The invention concerns compounds of formula (I) having antiviral activity, in particular, having an inhibitory activity on the replication of the respiratory syncytial virus (RSV). The invention further concerns pharmaceutical compositions comprising these compounds and the compounds for use in the treatment of respiratory syncytial virus infection.

### FIELD OF THE INVENTION

The invention concerns compounds having antiviral activity, in particular having an inhibitory activity on the replication of the respiratory syncytial virus (RSV). The invention further concerns pharmaceutical compositions comprising these compounds and the compounds for use in the treatment of respiratory syncytial virus infection.

### BACKGROUND

Human RSV or Respiratory Syncytial Virus is a large RNA virus, member of the family of Pneumoviridae, genus Orthopneumovirus together with bovine RSV virus. Human RSV is responsible for a spectrum of respiratory tract diseases in people of all ages throughout the world. It is the major cause of lower respiratory tract illness during infancy and childhood. Over half of all infants encounter RSV in their first year of life, and almost all within their first two years. The infection in young children can cause lung damage that persists for years and may contribute to chronic lung disease in later life (chronic wheezing, asthma). Older children and adults often suffer from a (bad) common cold upon RSV infection. In old age, susceptibility again increases, and RSV has been implicated in a number of outbreaks of pneumonia in the aged resulting in significant mortality.

Infection with a virus from a given subgroup does not protect against a subsequent infection with an RSV isolate from the same subgroup in the following winter season. Re-infection with RSV is thus common, despite the existence of only two subtypes, A and B.

Today only two drugs have been approved for use against RSV infection. A first one is ribavirin, a nucleoside analogue that provides an aerosol treatment for serious RSV infection in hospitalized children. The aerosol route of administration, the toxicity (risk of teratogenicity), the cost and the highly variable efficacy limit its use. Synagis® (palivizumab a monoclonal antibody, is used for passive immunoprophylaxis. Although the benefit of Synagis® has been demonstrated, the treatment is expensive, requires parenteral administration and is restricted to children at risk for developing severe pathology.

Clearly there is a need for an efficacious non-toxic and easy to administer drug against RSV replication. It would be particularly preferred to provide drugs against RSV replication that could be administered perorally.

Compounds that exhibit anti-RSV activity are disclosed in WO-2016/174079.

### DETAILED DESCRIPTION OF THE INVENTION

The present invention relates to compounds of formula (I)

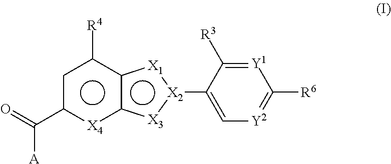

including any stereochemically isomeric form thereof, wherein
   A is

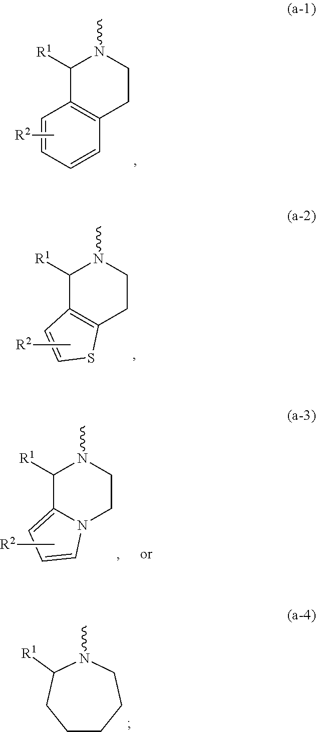

X₁, X₂, X₃, and X₄ are each independently selected from C, CH, N, NR⁵, 0 or S with the proviso that none of X₁, X₂, X₃, and X₄ are all C or CH;
Y¹ and Y² are each independently selected from CH, CF and N;
R¹ is CH₃ or CH₂CH₃;
R² is hydrogen, halo or C₁₋₄alkyl;
R³ is halo;
R⁴ is C₁₋₆alkyl; C₃₋₆cycloalkyl; di(C₁₋₄alkyl)amino; pyrrolidinyl; phenyl; pyridine; or phenyl or pyridine substituted with 1, 2 or 3 substituents each individually selected from halo, hydroxy, cyano, C₁₋₄alkyl, polyhaloC₁₋₄alkyl, and C₁₋₄alkyloxy;
R⁵ is hydrogen or C₁₋₄alkyl;
R⁶ is NH₂ or a substituent selected from substituent (a) or (b); wherein
   (a) is —NR⁷—(CO)-Heterocycle wherein said Heterocycle is substituted with one, two or three substituents each independently selected from halo, hydroxy, C₁₋₄alkyl of C₁₋₄alkyloxy; or
   (b) is C₃₋₆cycloalkyl or Heterocycle, wherein said C₃₋₆cycloalkyl and Heterocycle is substituted with one, two or three substituents each independently selected from
      C₁₋₆alkyl;
      C₁₋₆alkyl substituted with one, two or three substituents each independently selected from halo, hydroxy, hydroxycarbonyl, and aminocarbonyl;
      hydroxy;
      halo;
      —(CO)—OH;
      —(CO)—NR¹⁰R¹¹;
      —(CO)—NR⁸—SO₂—R⁹;
      —NR⁸R⁹;
      —NR⁸—(CO)—C₁₋₄alkyl;
      —NR⁸—(CO)—C₃₋₆cycloalkyl;
      —NR⁸—SO₂—R⁹;
      —SO₂—NR¹⁰R¹¹; or
      —SO₂—NR⁸—(CO)—R⁹;
      wherein
         R⁷ is hydrogen or C₁₋₄alkyl;
         each R⁸ is independently selected from hydrogen, C₁₋₄alkyl, or hydroxyC₁₋₄alkyl;
         R⁹ is C₁₋₄alkyl, polyhaloC₁₋₄alkyl, or C₃₋₆cycloalkyl;
         R¹⁰ and R¹¹ are each independently selected from hydrogen; C₁₋₄alkyl;
         polyhaloC₁₋₄alkyl; C₃₋₆cycloalkyl; C₃₋₆cycloalkyl substituted with C₁₋₄alkyl; or C₁₋₄alkyl substituted with hydroxy or cyano;
            Heterocycle is azetidinyl, pyrrolodinyl, piperidinyl, or homopiperidinyl;
            or a pharmaceutically acceptable acid addition salt thereof.

As used in the foregoing definitions:
halo is generic to fluoro, chloro, bromo and iodo;
C₁₋₄alkyl defines straight and branched chain saturated hydrocarbon radicals having from 1 to 4 carbon atoms such as, for example, methyl, ethyl, propyl, butyl, 1-methylethyl, 2-methyl-propyl and the like;
C₁₋₆alkyl is meant to include C₁₋₄alkyl and the higher homologues thereof having 5 or 6 carbon atoms, such as, for example, 2 methylbutyl, pentyl, hexyl and the like;
C₃₋₆cycloalkyl is generic to cyclopropyl, cyclobutyl, cyclopentyl, and cyclohexyl;
polyhaloC₁₋₄alkyl is defined as polyhalosubstituted C₁₋₄alkyl, in particular C₁₋₄alkyl (as hereinabove defined) substituted with 2 to 6 halogen atoms such as difluoromethyl, trifluoromethyl, trifluoroethyl, and the like;
—(CO)— or (CO) means carbonyl.

The term “compounds of the invention” as used herein, is meant to include the compounds of formula (I), and the salts and solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the terms “compound of formula (I)” and “intermediates of synthesis of formula (I)” are meant to include the stereoisomers thereof and the tautomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers. Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. Substituents on bivalent cyclic (partially) saturated radicals may have either the cis- or trans-configuration; for example, if a compound contains a disubstituted cycloalkyl group, the substituents may be in the cis or trans configuration.

The term “stereoisomers” also includes any rotamers, also called conformational isomers, the compounds of formula (I) may form.

Therefore, the invention includes enantiomers, diastereomers, racemates, E isomers, Z isomers, cis isomers, trans isomers, rotamers, and mixtures thereof, whenever chemically possible.

The meaning of all those terms, i.e. enantiomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

Some of the compounds according to formula (I) may also exist in their tautomeric form. Such forms in so far as they may exist, although not explicitly indicated in the above formula (I) are intended to be included within the scope of the present invention.

It follows that a single compound may exist in both stereoisomeric and tautomeric form.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

Conversely said salt forms can be converted by treatment with an appropriate base into the free base form.

The compounds of formula (I) may exist in both unsolvated and solvated forms. The term ‘solvate’ is used herein to describe a molecular association comprising a compound of the invention and one or more pharmaceutically acceptable solvent molecules, e.g. water or ethanol. The term ‘hydrate’ is used when said solvent is water.

For the avoidance of doubt, compounds of formula (I) may contain the stated atoms in any of their natural or non-natural isotopic forms. In this respect, embodiments of the invention that may be mentioned include those in which (a) the compound of formula (I) is not isotopically enriched or labelled with respect to any atoms of the compound; and (b) the compound of formula (I) is isotopically enriched or labelled with respect to one or more atoms of the compound. Compounds of formula (I) that are isotopically enriched or labelled (with respect to one or more atoms of the compound) with one or more stable isotopes include, for example, compounds of formula (I) that are isotopically enriched or labelled with one or more atoms such as deuterium, ¹³C, ¹⁴C, ¹⁴N, ¹⁵O or the like.

A first group of compounds are compounds of formula (I) wherein X₁, X₂, X₃, and X₄ are selected from

| X₁ | X₂ | X₃ | X₄ | |
|---|---|---|---|---|
| CH | C | NR⁵ | CH | (b-1) |
| N | C | NR⁵ | CH | (b-2) |
| N | C | NR⁵ | N | (b-3) |
| NR⁵ | C | N | N | (b-4) |
| CH | N | N | CH | (b-5) |
| N | N | CH | CH | (b-6) |
| N | C | O | CH | (b-7) |
| N | N | CH | N | (b-8) |
| N | C | S | CH | (b-9) |
| O | C | CH | N | (b-10) |
| N | C | O | N | (b-11) |
| O | C | CH | CH | (b-12) |
| CH | C | S | N | (b-13) |
| S | C | N | N | (b-14). |

A second group of compounds are compounds of formula (I) wherein radical A is of formula (a-1).

A third group of compounds are compounds of formula (I) wherein R⁶ is a substituent (a).

A fourth group of compounds are compounds of formula (I) wherein R⁶ is a substituent (b).

A fifth group of compounds are compounds of formula (I) wherein Y¹ and Y² are each independently selected from CH.

Interesting compounds of formula (I) are those compounds of formula (I) wherein one or more of the following restrictions apply:
a) A is a radical of formula (a-1) wherein R¹ is CH₃; or
b) R² is hydrogen; or
c) R³ is fluoro; or
d) R⁴ is C₃₋₆cycloalkyl, in particular cyclopropyl; or
e) R⁴ is C₁₋₄alkyl, in particular ethyl; or
f) R⁴ is phenyl; or
g) R⁶ is substituent (a) of formula —NR⁷—(CO)-Heterocycle wherein said Heterocycle is pyrrolidinyl substituted with hydroxy; or
h) R⁶ is substituent (b) and substituent (b) is C₃₋₆cycloalkyl substituted with one or two substituents each independently selected from —(CO)—OH or —(CO)—NR¹⁰R¹¹ wherein R¹⁰ and R¹¹ are each hydrogen; and
i) R⁶ is substituent (b) and substituent (b) is Heterocycle wherein said Heterocycle is pyrrolidinyl substituted with one or two substituents each independently selected from hydroxy, —(CO)—OH or —(CO)—NR¹⁰R¹¹ wherein R¹⁰ and R¹¹ are each hydrogen.

In an embodiment, the present invention relates to compounds of formula (I)
including any stereochemically isomeric form thereof, wherein

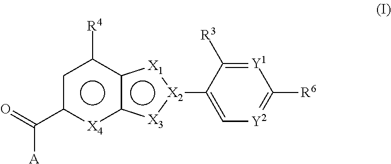

A is

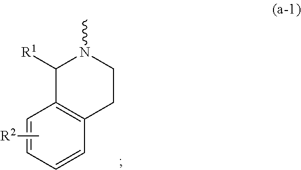

X₁, X₂, X₃, and X₄ are selected from

| X₁ | X₂ | X₃ | X₄ | |
|---|---|---|---|---|
| CH | C | NR⁵ | CH | (b-1) |
| N | C | NR⁵ | CH | (b-2) |
| N | C | NR⁵ | N | (b-3) |
| NR⁵ | C | N | N | (b-4) |
| CH | N | N | CH | (b-5) |
| N | N | CH | CH | (b-6) |
| N | C | O | CH | (b-7) |
| N | N | CH | N | (b-8) |
| N | C | S | CH | (b-9) |
| O | C | CH | N | (b-10) |
| N | C | O | N | (b-11) |
| O | C | CH | CH | (b-12) |
| CH | C | S | N | (b-13) |
| S | C | N | N | (b-14); |

Y¹ and Y² are each independently selected from CH;
R¹ is CH₃;
R² is hydrogen;
R³ is halo;
R⁴ is C₁₋₆alkyl, C₃₋₆cycloalkyl, or phenyl;
R⁵ is hydrogen or C₁₋₄alkyl;
R⁶ is NH₂ or a substituent selected from substituent (a) or (b); wherein
   (a) is —NR⁷—(CO)-Heterocycle wherein said Heterocycle is substituted with hydroxy and R⁷ is hydrogen; or
   (b) is C₃₋₆cycloalkyl or Heterocycle, wherein said C₃₋₆cycloalkyl and Heterocycle is substituted with one or two substituents each independently selected from hydroxy, —(CO)—OH or —(CO)—NR¹⁰R¹¹ wherein R¹⁰ and R¹¹ are each hydrogen; and
      Heterocycle is pyrrolodinyl;
      or a pharmaceutically acceptable acid addition salt thereof.

In general compounds of formula (I) can be prepared by reacting an intermediate of formula (II) with an alkylboronate intermediate of formula (III) in at least one reaction-inert solvent and optionally in the presence of at least one transition metal coupling reagent and/or at least one suitable ligand, the said process further optionally comprising converting a compound of formula (I) into an addition salt thereof. Suitable metal coupling reagents and/or suitable ligands for this reaction are, e.g. palladium compounds such as palladium tetra(triphenylphosphine), tris(dibenzylidene-acetone dipalladium, 2,2′-bis(diphenylphosphino)-1,1′-binaphtyl and the like.

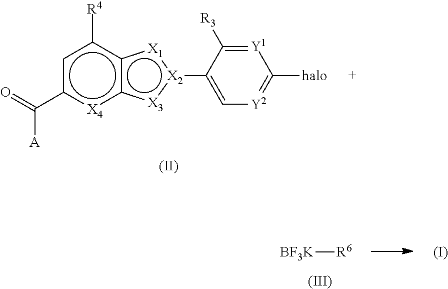

Compounds of formula (I) can generally also be prepared by reacting an intermediate of formula (IV) with an intermediate of formula (V) in a reaction-inert solvent, such as dichloromethane or DMF, in the present of a suitable reagent, such as HATU (1-[bis(dimethylamino)methylene]-1H-1,2,3-triazolo[4,5-b]-pyridinium 3-oxid hexafluorophosphate), and a base such as triethylamine.

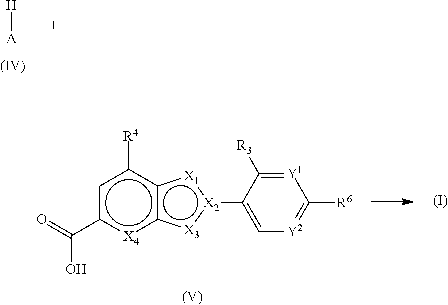

Compounds of formula (I) can also be prepared by reacting an intermediate of formula (VI) with an intermediate of formula (VII) in a reaction-inert solvent and optionally in the presence of at least one transition metal coupling reagent and/or at least one suitable ligand.

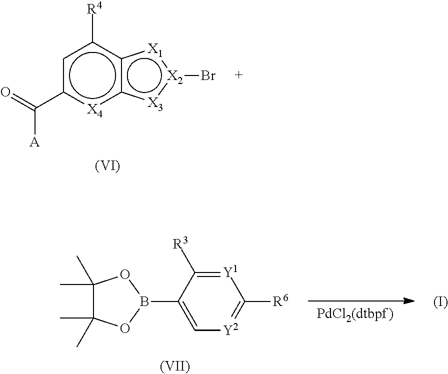

Other synthetic pathways for preparing compounds of formula (I) have been described in the experimental party as general methods of preparation and specific working examples.

The compounds of formula (I) may further be prepared by converting compounds of formula (I) into each other according to art-known group transformation reactions.

The starting materials and some of the intermediates are known compounds and are commercially available or may be prepared according to conventional reaction procedures generally known in the art.

The compounds of formula (I) as prepared in the hereinabove described processes may be synthesized in the form of racemic mixtures of enantiomers which can be separated from one another following art-known resolution procedures. Those compounds of formula (I) that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

The compounds of formula (I) show antiviral properties. Viral infections treatable using the compounds and methods of the present invention include those infections brought on by ortho- and paramyxoviruses and in particular by human and bovine respiratory syncytial virus (RSV). A number of the compounds of this invention moreover are active against mutated strains of RSV. Additionally, many of the compounds of this invention show a favorable pharmacokinetic profile and have attractive properties in terms of bioavailability, including an acceptable half-life, AUC and peak values and lacking unfavourable phenomena such as insufficient quick onset and tissue retention.

The in vitro antiviral activity against RSV of the present compounds was tested in a test as described in the experimental part of the description, and may also be demonstrated in a virus yield reduction assay. The in vivo antiviral activity against RSV of the present compounds may be demonstrated in a test model using cotton rats as described in Wyde et al. in Antiviral Research, 38, p. 31-42 (1998).

Additionally the present invention provides pharmaceutical compositions comprising at least one pharmaceutically acceptable carrier and a therapeutically effective amount of a compound of formula (I). Also provided are pharmaceutical compositions comprising a pharmaceutically acceptable carrier, a therapeutically active amount of a compound of formula (I), and another antiviral agent, in particular a RSV inhibiting compound.

In order to prepare the pharmaceutical compositions of this invention, an effective amount of the particular compound, in base or acid addition salt form, as the active ingredient is combined in intimate admixture with at least one pharmaceutically acceptable carrier, which carrier may take a wide variety of forms depending on the form of preparation desired for administration. These pharmaceutical compositions are desirably in unitary dosage form suitable, preferably, for oral administration, rectal administration, percutaneous administration or parenteral injection.

For example in preparing the compositions in oral dosage form, any of the usual liquid pharmaceutical carriers may be employed, such as for instance water, glycols, oils, alcohols and the like in the case of oral liquid preparations such as suspensions, syrups, elixirs and solutions; or solid pharmaceutical carriers such as starches, sugars, kaolin, lubricants, binders, disintegrating agents and the like in the case of powders, pills, capsules and tablets. Because of their easy administration, tablets and capsules represent the most advantageous oral dosage unit form, in which case solid pharmaceutical carriers are obviously employed. For parenteral injection compositions, the pharmaceutical carrier will mainly comprise sterile water, although other ingredients may be included in order to improve solubility of the active ingredient. Injectable solutions may be prepared for instance by using a pharmaceutical carrier comprising a saline solution, a glucose solution or a mixture of both. Injectable suspensions may also be prepared by using appropriate liquid carriers, suspending agents and the like. In compositions suitable for percutaneous administration, the pharmaceutical carrier may optionally comprise a penetration enhancing agent and/or a suitable wetting agent, optionally combined with minor proportions of suitable additives which do not cause a significant deleterious effect to the skin. Said additives may be selected in order to facilitate administration of the active ingredient to the skin and/or be helpful for preparing the desired compositions. These topical compositions may be administered in various ways, e.g., as a transdermal patch, a spot-on or an ointment. Addition salts of the compounds of formula (I), due to their increased water solubility over the corresponding base form, are obviously more suitable in the preparation of aqueous compositions.

It is especially advantageous to formulate the pharmaceutical compositions of the invention in dosage unit form for ease of administration and uniformity of dosage. “Dosage unit form” as used herein refers to physically discrete units suitable as unitary dosages, each unit containing a predetermined amount of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

For oral administration, the pharmaceutical compositions of the present invention may take the form of solid dose forms, for example, tablets (both swallowable and chewable forms), capsules or gelcaps, prepared by conventional means with pharmaceutically acceptable excipients and carriers such as binding agents (e.g. pregelatinised maize starch, polyvinylpyrrolidone, hydroxypropylmethylcellulose and the like), fillers (e.g. lactose, microcrystalline cellulose, calcium phosphate and the like), lubricants (e.g. magnesium stearate, talc, silica and the like), disintegrating agents (e.g. potato starch, sodium starch glycollate and the like), wetting agents (e.g. sodium laurylsulphate) and the like. Such tablets may also be coated by methods well known in the art.

Liquid preparations for oral administration may take the form of e.g. solutions, syrups or suspensions, or they may be formulated as a dry product for admixture with water and/or another suitable liquid carrier before use. Such liquid preparations may be prepared by conventional means, optionally with other pharmaceutically acceptable additives such as suspending agents (e.g. sorbitol syrup, methylcellulose, hydroxypropylmethylcellulose or hydrogenated edible fats), emulsifying agents (e.g. lecithin or acacia), non aqueous carriers (e.g. almond oil, oily esters or ethyl alcohol), sweeteners, flavours, masking agents and preservatives (e.g. methyl or propyl p-hydroxybenzoates or sorbic acid).

Pharmaceutically acceptable sweeteners useful in the pharmaceutical compositions of the invention comprise preferably at least one intense sweetener such as aspartame, acesulfame potassium, sodium cyclamate, alitame, a dihydrochalcone sweetener, monellin, stevioside sucralose (4,1′,6′-trichloro-4,1′,6′-trideoxygalactosucrose) or, preferably, saccharin, sodium or calcium saccharin, and optionally at least one bulk sweetener such as sorbitol, mannitol, fructose, sucrose, maltose, isomalt, glucose, hydrogenated glucose syrup, xylitol, caramel or honey. Intense sweeteners are conveniently used in low concentrations. For example, in the case of sodium saccharin, the said concentration may range from about 0.04% to 0.1% (weight/volume) of the final formulation. The bulk sweetener can effectively be used in larger concentrations ranging from about 10% to about 35%, preferably from about 10% to 15% (weight/volume).

The pharmaceutically acceptable flavours which can mask the bitter tasting ingredients in the low-dosage formulations are preferably fruit flavours such as cherry, raspberry, black currant or strawberry flavour. A combination of two flavours may yield very good results. In the high-dosage formulations, stronger pharmaceutically acceptable flavours may be required such as Caramel Chocolate, Mint Cool, Fantasy and the like. Each flavour may be present in the final composition in a concentration ranging from about 0.05% to 1% (weight/volume). Combinations of said strong flavours are advantageously used. Preferably a flavour is used that does not undergo any change or loss of taste and/or color under the circumstances of the formulation.

The compounds of formula (I) may be formulated for parenteral administration by injection, conveniently intravenous, intra-muscular or subcutaneous injection, for example by bolus injection or continuous intravenous infusion. Formulations for injection may be presented in unit dosage form, e.g. in ampoules or multi-dose containers, including an added preservative. They may take such forms as suspensions, solutions or emulsions in oily or aqueous vehicles, and may contain formulating agents such as isotonizing, suspending, stabilizing and/or dispersing agents. Alternatively, the active ingredient may be present in powder form for mixing with a suitable vehicle, e.g. sterile pyrogen free water, before use.

The compounds of formula (I) may also be formulated in rectal compositions such as suppositories or retention enemas, e.g. containing conventional suppository bases such as cocoa butter and/or other glycerides.

In general it is contemplated that an antivirally effective daily amount would be from 0.01 mg/kg to 500 mg/kg body weight, more preferably from 0.1 mg/kg to 50 mg/kg body weight. It may be appropriate to administer the required dose as two, three, four or more sub-doses at appropriate intervals throughout the day. Said sub-doses may be formulated as unit dosage forms, for example, containing 1 to 1000 mg, and in particular 5 to 200 mg of active ingredient per unit dosage form.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention. The effective daily amount ranges mentioned hereinabove are therefore only guidelines.

Also, the combination of another antiviral agent and a compound of formula (I) can be used as a medicine. Thus, the present invention also relates to a product containing (a) a compound of formula (I), and (b) another antiviral compound, as a combined preparation for simultaneous, separate or sequential use in antiviral treatment. The different drugs may be combined in a single preparation together with pharmaceutically acceptable carriers. For instance, the compounds of the present invention may be combined with interferon-beta or tumor necrosis factor-alpha in order to treat or prevent RSV infections. Other antiviral compounds (b) to be combined with a compound of formula (I) for use in the treatment of RSV are RSV fusion inhibitors or RSV polymerase inhibitors. Specific antiviral compounds for combination with any of the compounds of formula (I) that are useful in the treatment of RSV are the RSV inhibiting compounds selected from ribavirin, lumicitabine, presatovir, ALX-0171, MDT-637, BTA-9881, BMS-433771, YM-543403, A-60444, TMC-353121, RFI-641, CL-387626, MBX-300, sisunatovir, ziresovir, 3-({5-chloro-1-[3-(methyl-sulfonyl)propyl]-1H-benzimidazol-2-yl}methyl)-1-cyclopropyl-1,3-dihydro-2H-imidazo[4,5-c]pyridin-2-one, 3-[[7-chloro-3-(2-ethylsulfonyl-ethyl)imidazo[1,2-a]pyridin-2-yl]methyl]-1-cyclopropyl-imidazo[4,5-c]pyridin-2-one, and 3-({5-chloro-1-[3-(methyl-sulfonyl)propyl]-1H-indol-2-yl}methyl)-1-(2,2,2-trifluoroethyl)-1,3-dihydro-2H-imidazo[4,5-c]pyridin-2-one.

### Experimental Part

### A. Abbreviations

| | |
|---|---|
| μw | microwave |
| AcOH | acetic acid |
| NaOAc | sodium acetate |
| aq. | aqueous |
| B₂Pin₂ | bis pinacolato diboron cas [73183-34-3] |
| boc | di-tert-butylcarbonate |
| br | broad |
| cataCXium ® A | di(1-adamantyl)-n-butylphosphine |
| | CAS [321921-71-5] |
| CDI | 1,1′-carbonyldiimidazole |
| | CAS [530-62-1] |
| COMU ® | 1-cyano-2-ethoxy-2-oxoethylidenaminooxy)dimeth- |
| | ylamino-morpholino-carbenium hexafluorophos- |
| | phate CAS: [1075198-30-9] |
| CPME | cyclopentyl methyl ether |
| CyJohnPhos | 2-(dicyclohexylphosphino)biphenyl |
| | CAS: [247940-06-3] |
| d | doublet |
| DCE | dichloroethane |
| DCM | dichloromethane |
| DIPEA | N,N-diisopropylethylamine |
| DMEDA | N,N′-dimethylethylenediamine |
| DMF | dimethylformamide |
| DMSO | dimethyl sulfoxide |
| Et₂O | diethyl ether |
| Et₃N/TEA | triethylamine |
| EtOAc | ethyl acetate |
| EtOH | ethanol |
| h | hour |
| HATU | 1-[bis(dimethylamino)methylene]-1h- |
| | 1,2,3-triazolo[4,5-b]-pyridinium |
| | 3-oxid hexafluorophosphate |
| | CAS [148893-10-1] |
| LiHMDS | lithium bis(trimethylsilyl)amide |
| m | multiplet |
| m/z | mass-to-charge ratio |
| mCPBA | 3-chloroperbenzoic acid |
| MeCN | acetonitrile |
| MeOH | methanol |
| Me-THF | 2-methyltetrahydrofuran |
| min | minute(s) |
| MTBE | tert-butyl methyl ether |
| NBS | N-bromosuccinimide |
| NIS | N-iodosuccinimide |
| NMP | N-methyl-2-pyrrolidone |
| NMR | nuclear magnetic resonance |
| o/n | overnight |
| Pd118 | 1,1′-bis (di-t-butylphosphino)ferrocene |
| | palladium dichloride CAS [95408-45-0] |
| Pd₂dba₃ | tris(dibenzylideneacetone)dipalladium(0) |
| | CAS [51364-51-3] |
| Pd(OAc)₂ | palladium (II) acetate |
| | CAS [3375-31-3] |
| PdCl₂(dppf) | [1,1′-bis(diphenylphosphino)ferrocene]dichloro- |
| | palladium(II) CAS [72287-26-4] |
| PdCl₂(dppf)•DCM | [1,1′-bis(diphenylphosphino)ferrocene]dichloro- |
| | palladium(II), complex with dichloromethane |
| | CAS [95464-05-4] |
| PdCl₂(PPh₃)₂ | bis(triphenylphosphine)palladium(II) dichloride |
| | CAS [13965-03-2] |
| PPACA/T3P | propylphosphonic anhydride |
| | CAS [68957-94-8] |
| PPh₃ | triphenylphosphine |
| ppm | parts per million |
| q | quartet |
| quin | quintuplet |
| rt | room temperature |
| RuPhos | 2-dicyclohexylphosphino-2′,6′-diisopropoxybiphenyl |
| | CAS [787618-22-8] |
| s | singulet |
| sext | sextuplet |
| t | triplet |
| t-BuOK | potassium tert-butoxide |
| THF | tetrahydrofuran |
| TMS | trimethylsilyle |
| XantPhos | 4,5-bis(diphenylphosphino)-9,9-dimethylxanthene |
| | CAS [161265-03-8] |
| Xphos | 2-dicyclohexylphosphino-2′,4′,6′-triisopro- |
| | pylbiphenyl CAS [564483-18-7] |
| Δ | heat |
| DavePhos | 2-dicyclohexylphosphino-2-(N,N-dimeth- |
| | ylamino)biphenyl |

The stereochemical configuration for some compounds has been designated as R* or S* (or *R or *S) when the absolute stereochemistry is undetermined (even if the bonds are drawn stereospecifically) although the compound itself has been isolated as a single stereoisomer and is enantiomerically pure. This means that the absolute stereoconfiguration of the stereocenter indicated by * is undetermined (even if the bonds are drawn stereospecifically) although the compound is enantiomerically pure at the indicated centre.

### B. Compound Synthesis

### 1. Synthesis of Oxazolopyridine

### 1.1 Synthesis of Compound 1

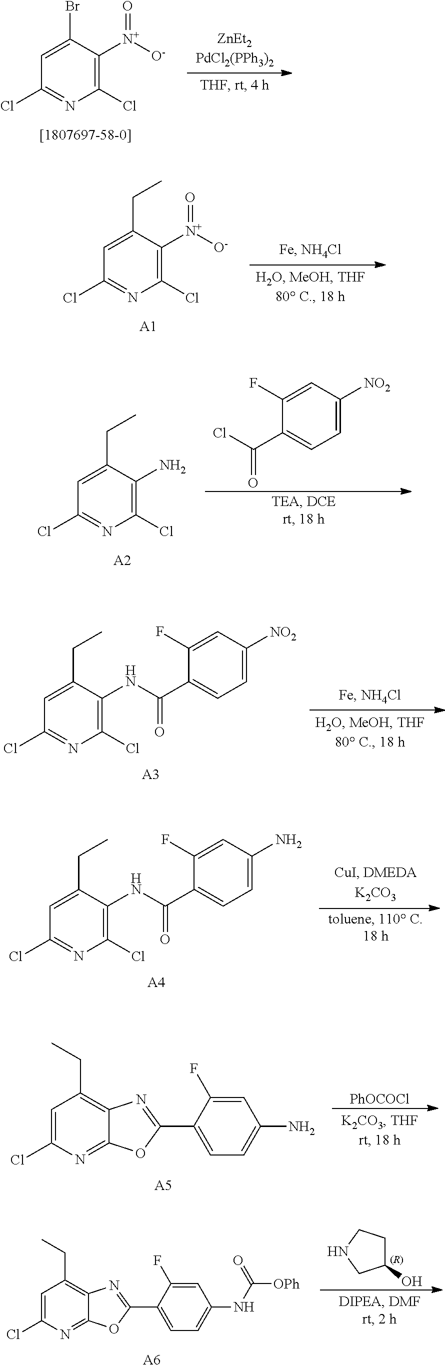

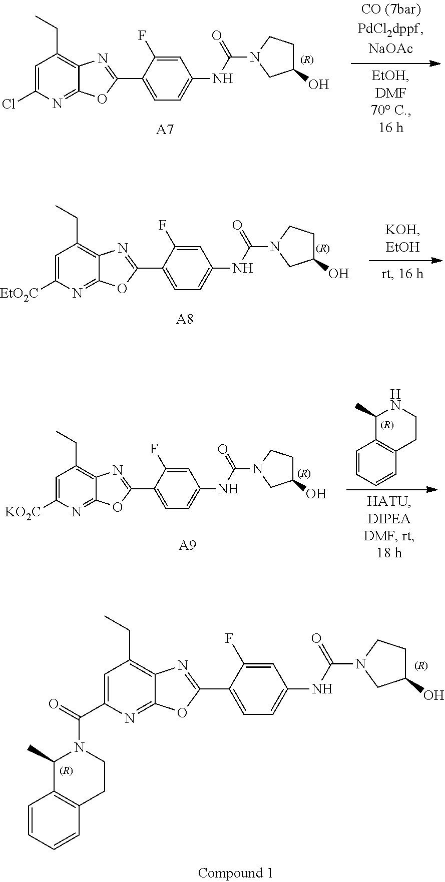

### Intermediate A1

### 2,6-dichloro-4-ethyl-3-nitropyridine

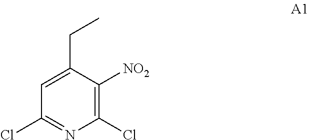

Diethylzinc 15% in toluene (4.4 mL, 4.9 mmol) was added to a solution of 4-bromo-2,6-dichloro-3-nitropyridine (1.33 g, 4.89 mmol) in THF (30 mL). The mixture was purged with N₂. PdCl₂(PPh₃)₂ (343 mg, 0.489 mmol) was added. The mixture was purged with N₂ and stirred at rt for 4 h. An extraction was performed with EtOAc and water. The organic layer was washed with brine, dried on MgSO₄, evaporated to dryness. The residue was purified by preparative LC (irregular SiOH 15-40 μm, 40 g GraceResolv®, mobile phase gradient: from heptane/EtOAc 99/1 to 50/50). The fractions containing product were combined and evaporated under vacuum to give intermediate A1 (922 mg, 85%).

### Intermediate A2

### 2,6-dichloro-4-ethylpyridin-3-amine

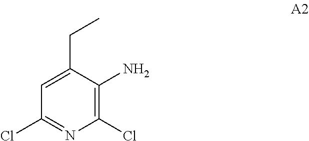

In a sealed tube, a solution of intermediate A1 (922 mg, 4.17 mmol), iron (1.17 g, 20.9 mmol), ammonium chloride (2.23 g, 41.8 mmol) in THE (13 mL), MeOH (13 mL) and H₂O (6.6 mL) was heated at 80° C. for 18 h. The mixture was cooled down to rt then diluted in EtOAc and water. The layers were separated and the organic layer was washed with brine, dried on MgSO₄, filtered and evaporated to give a brown oil which was purified by preparative LC (irregular SiOH, 15-40 μm, GraceResolv® 40 g, mobile phase gradient: from heptane/EtOAc 99/01 to 50/50). The fractions containing product were combined and evaporated under vacuum to give intermediate A2 (538 mg, 68%) as a colorless oil.

### Intermediate A3

### N-(2,6-dichloro-4-ethylpyridin-3-yl)-2-fluoro-4-nitrobenzamide

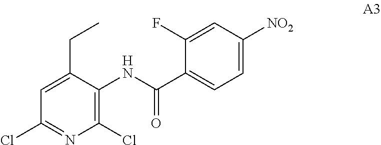

2-Fluoro-4-nitrobenzoyl chloride (688 mg, 3.38 mmol) was added to a mixture of intermediate A2 (497 mg, 2.60 mmol) and TEA (0.542 mL, 3.90 mmol) in DCE (17 mL) at 0° C. The resulting mixture was stirred at rt for 18 h. The solvent was removed under vacuum and the residue was taken-up with DCM, the solid was filtrated over frit to give intermediate A3 (777 mg, 83%) as a yellow solid.

### Intermediate A4

### 4-amino-N-(2,6-dichloro-4-ethylpyridin-3-yl)-2-fluorobenzamide

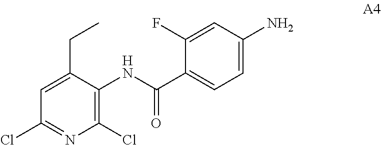

In a sealed tube, a solution of intermediate A3 (308 mg, 0.86 mmol), iron (0.24 g, 4.3 mmol), ammonium chloride (0.461 g, 8.61 mmol) in THE (2.7 mL), MeOH (2.7 mL) and H₂O (1.4 mL) was heated at 80° C. for 18 h. The mixture was cooled down to rt then diluted in EtOAc and water. The layers were separated and the organic layer was washed with brine, dried on MgSO₄, filtered and evaporated to give intermediate A4 (285 mg, quant.) as a white solid.

### Intermediate A5

### 4-(5-chloro-7-ethyloxazolo[5,4-b]pyridin-2-yl)-3-fluoroaniline

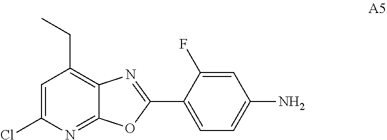 [SMILES: CCc1cc(Cl)nc2oc(-c3ccc(N)cc3F)nc12]

A mixture of intermediate A4 (555 mg, 1.69 mmol) and K₂CO₃ (701 mg, 5.07 mmol) in toluene (13.5 mL). The mixture was purged with N₂. Then DMEDA (218 μL, 2.03 mmol) and Cut (354 mg, 1.86 mmol) was added and the reaction mixture was stirred at 110° C. for 18 h. The mixture was cooled down to rt then filtered over Celite® and the filtrate was evaporated to dryness. The residue was purified by preparative LC (Irregular SiOH 15-40 μm, 24 g GraceResolv®, mobile phase: from Heptane/EtOAc 70/30 to 20/80). The fractions containing product were combined and evaporated under vacuum to give intermediate A5 (356 mg, 72%).

### Intermediate A6

### phenyl (4-(5-chloro-7-ethyloxazolo[5,4-b]pyridin-2-yl)-3-fluorophenyl)carbamate

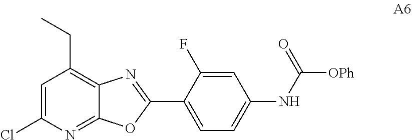 [SMILES: CCc1cc(Cl)nc2oc(-c3ccc(NC(=O)Oc4ccccc4)cc3F)nc12]

In a mixture of intermediate A5 (356 mg, 1.22 mmol) and K₂CO₃ (388 mg, 2.81 mmol) in THE (15 mL) was added Phenyl chloroformate (0.153 mL, 1.22 mmol). The mixture was stirred at rt for 18 h. Water and EtOAc were added and an extraction was performed with EtOAc (twice). The combined organic layers were dried over MgSO₄, filtered and concentrated in vacuo to give intermediate A6 (499 mg, 99%).

### Intermediate A7

### (R)-N-(4-(5-chloro-7-ethyloxazolo[5,4-b]pyridin-2-yl)-3-fluorophenyl)-3-hydroxypyrrolidine-1-carboxamide

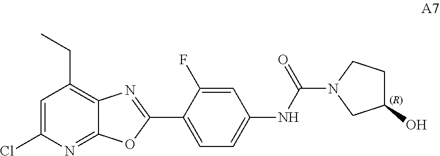

To a stirred mixture of intermediate A6 (475 mg, 1.15 mmol) and (R)-(+)-3-Pyrrolidinol (151 mg, 1.73 mmol) in DMF (26 mL) was added DIPEA (994 μL, 5.77 mmol). The reaction mixture was stirred at rt for 2 h. Solvent was removed under vacuum. Water and EtOAc were added. The aqueous layer was extracted with EtOAc (twice), the combined organic layers were dried over MgSO₄, filtered and concentrated in vacuo. The residue was purified by preparative LC (irregular SiOH 15-40 μm, 24 g GraceResolv®, mobile phase gradient: from DCM/MeOH 99/1 to 90/10). The fractions containing product were combined and evaporated under vacuum to give intermediate A7 as a white solid (420 mg, 90%).

### Intermediate A8

### Ethyl (R)-7-ethyl-2-(2-fluoro-4-(3-hydroxypyrrolidine-1-carboxamido)phenyl)oxazolo[5,4-b]pyridine-5-carboxylate

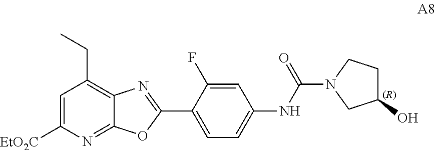

In a pressure vessel reactor, to a degassed mixture of intermediate A7 (0.42 g, 1.04 mmol) and sodium acetate (170 mg, 2.08 mmol) in EtOH (6.2 mL) and DMF (2.7 mL) was added PdCl₂(dppf) (78 mg, 0.10 mmol) then the resulting mixture was stirred under 7 bars of CO. The resulting mixture was heated at 70° C. for 16 h. The mixture was cooled down to rt then water and EtOAc were added. The layers were separated and the aqueous layer was extracted with EtOAc (once). The combined organic layers were washed with brine, dried over MgSO₄, filtered and the solvent was removed in vacuo. The residue was purified by preparative LC (irregular SiOH, 15-40 μm, GraceResolv® 24 g, mobile phase gradient: from DCM/MeOH 99/1 to 90/10). The fractions containing product were combined and evaporated under vacuum to give intermediate A8 (272 mg, 59%).

### Intermediate A9

### Potassium (R)-7-ethyl-2-(2-fluoro-4-(3-hydroxypyrrolidine-1-carboxamido)phenyl)oxazolo[5,4-b]pyridine-5-carboxylate

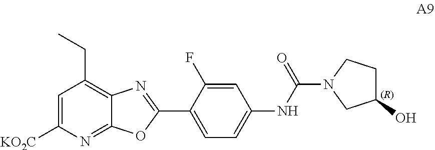

A mixture of intermediate A8 (272 mg, 0.615 mmol) and potassium hydroxide (76 mg, 1.3 mmol) in EtOH (5.3 mL) was stirred at rt for 16 h. The precipitate was filtered and dried over frit to give intermediate A9 as potassium salt (168 mg, 60%).

### Compound 1

### (R)-N-(4-(7-ethyl-5-((R)-1-methyl-1,2,3,4-tetrahydroisoquinoline-2-carbonyl)oxazolo[5,4-b]pyridin-2-yl)-3-fluorophenyl)-3-hydroxypyrrolidine-1-carboxamide

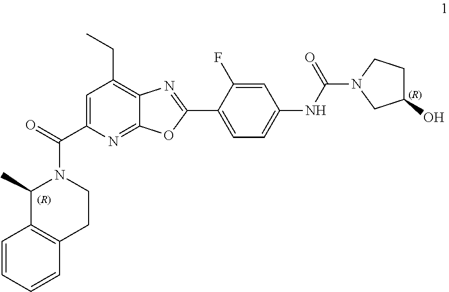

A mixture of intermediate A9 (149 mg, 0.329 mmol), R-(1)-methyl-(1,2,3,4)-tetrahydroisoquinoline (163 mg, 0.428 mmol), DIPEA (0.17 mL, 0.99 mmol) and HATU (58 mg, 0.40 mmol) in DMF (1.9 mL) was stirred at rt for 18 h. Water and EtOAc were added to the reaction mixture. The layers were separated. The aqueous layer was extracted twice with EtOAc. The combined organic layers were washed with brine (4 times), dried over MgSO₄ and evaporated in vacuo to give a brownish solid which was taken-up in MeCN. The precipitate obtained was filtrated and dried under vacuum at 50° C. for 6 h. The solid was purified by preparative LC (spherical C18 25 μm, 40 g YMC-ODS-25, mobile phase gradient 0.2% aq. NH₄⁺HCO₃⁻/MeCN from 75:25 to 25:75). The fractions containing product were combined and evaporated then the resulting solid was taken-up in MeCN. The precipitate obtained was filtrated and dried under vacuum at 50° C. for 6 h to give compound 1 as a white solid (82 mg, 46%).

1.2 Synthesis of Compound 2

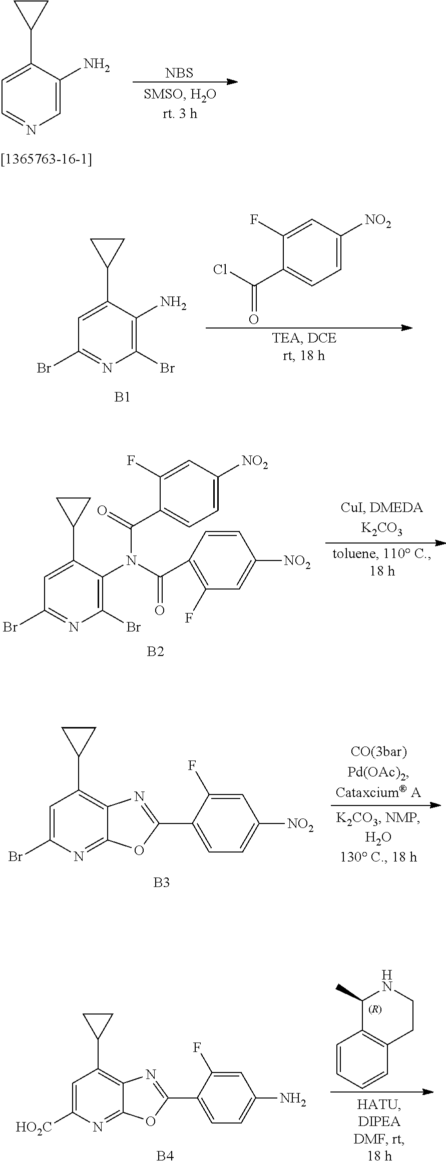

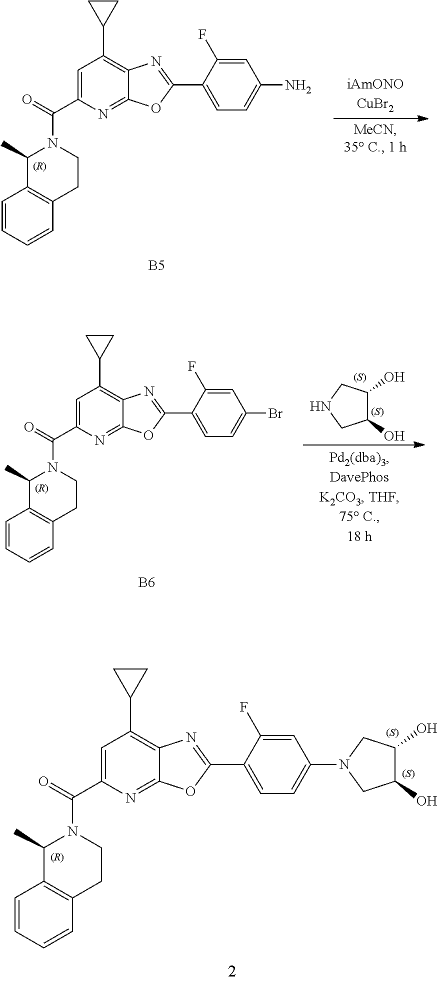

### Intermediate B1

### 2,6-dibromo-4-cyclopropylpyridin-3-amine

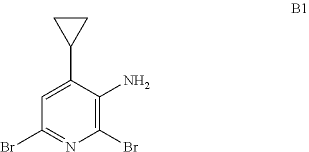 [SMILES: Nc1c(C2CC2)cc(Br)nc1Br]

NBS (0.558 g, 3.13 mmol) was added to a mixture of 3-amino-4-cyclopropylpyridine (200 mg, 1.49 mmol) in DMSO (3 mL) and H₂O (75 μL) at rt. The resulting mixture was stirred at rt for 3 h. water and EtOAc were added. The layers were separated. The aqueous layer was extracted with EtOAc/heptane. The combined organic layers were washed with water then aq. NaHCO₃ sat. (once), dried over MgSO₄, filtered and the solvent was removed in vacuo to give intermediate B1 (342 mg, 79%).

### Intermediate B2

### N-(2,6-dibromo-4-cyclopropylpyridin-3-yl)-2-fluoro-N-(2-fluoro-4-nitrobenzoyl)-4-nitrobenzamide

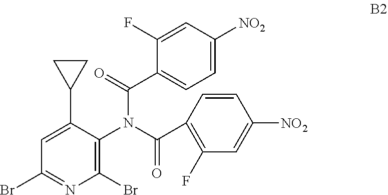 [SMILES: O=C(c1ccc([N+](=O)[O-])cc1F)N(C(=O)c1ccc([N+](=O)[O-])cc1F)c1c(C2CC2)cc(Br)nc1Br]

2-Fluoro-4-nitrobenzoyl chloride (663 mg, 3.26 mmol) was added to a mixture of intermediate B1 (732 mg, 2.51 mmol) and TEA (0.523 mL, 3.76 mmol) in DCE (17 mL) at 0° C. The resulting mixture was stirred at rt for 18 h. An extra amount of 2-Fluoro-4-nitrobenzoyl chloride (337 mg, 1.66 mmol) was added and the mixture was stirred at rt for 18 h. The solvent were removed under vacuum and the residue was purified by preparative LC (Regular SiOH 40 μm, 40 g Buchi®, mobile phase gradient: from Heptane/EtOAc 90/10 to 40/60). The fractions containing product were combined and evaporated under vacuum to give intermediate B2 (1.15 g, 73%).

### Intermediate B3

### 5-bromo-7-cyclopropyl-2-(2-fluoro-4-nitrophenyl)oxazolo[5,4-b]pyridine NO₂

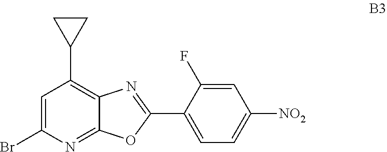 [SMILES: O=[N+]([O-])c1ccc(-c2nc3c(C4CC4)cc(Br)nc3o2)c(F)c1]

A mixture of intermediate B2 (1.15 g, 1.84 mmol) and potassium carbonate (0.76 g, 5.5 mmol) in toluene (15 mL). The mixture was purged with N₂. Then DMEDA (237 μL, 2.20 mmol) and CuI (385 mg, 2.02 mmol) was added and the reaction mixture was stirred at 110° C. for 18 h. The mixture was cooled down to rt then filtered over Celite® and evaporated to dryness. The residue was purified by preparative LC (Irregular SiOH 40 μm, 40 g Interchim®, mobile phase: from Heptane/EtOAc 90/10 to 40/60) The pure fraction was collected and evaporated to dryness to give intermediate B3 (363 mg, 52%).

### Intermediate B5

### (R)-(2-(4-amino-2-fluorophenyl)-7-cyclopropyloxazolo[5,4-b]pyridin-5-yl)(1-methyl-3,4-dihydroisoquinolin-2(1H)-yl)methanone

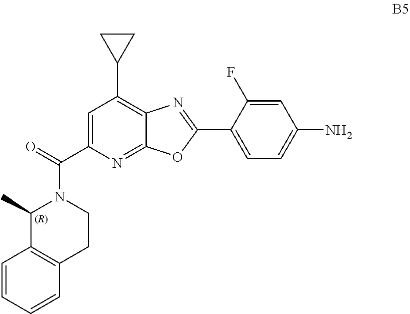

A degassed mixture of intermediate B3 (520 mg, 1.38 mmol) and K₂CO₃ (228 mg, 1.65 mmol) in NMP (14 mL) and H₂O (1.0 mL, 55 mmol) was carbonylated under 3 bar with Pd(OAc)₂ (31 mg, 0.14 mmol) and Cataxcium® A (99 mg, 0.28 mmol) as catalyst. The resulting mixture was stirred at 130° C. for 18 h. The mixture was cooled down to rt then the reaction mixture was filtered over silica and coevaporated three times with toluene to give a solution of crude intermediate B4 in NMP. To this solution was added HATU (680 mg, 1.79 mmol), DIPEA (711 μL, 4.13 mmol) and (1R)-methyl-(1,2,3,4)-tetrahydroisoquinoline (243 mg, 1.65 mmol) in DMF (7.9 mL). The resulting mixture was stirred at rt for 18 h. Water and EtOAc were added to the reaction mixture. The layers were separated. The aqueous layer was extracted twice with EtOAc. The combined organic layers were washed with brine, dried over MgSO₄ and evaporated in vacuo. The residue was purified by preparative LC (regular SiOH, 40 μm, 40 g Buchi®, mobile phase gradient: from heptane/EtOAc 80/20 to 20/80). The fractions containing product were combined and evaporated under vacuum to give intermediate B5 as a white solid (200 mg, 33% over 2 steps).

### Intermediate B6

### (R)-(2-(4-bromo-2-fluorophenyl)-7-cyclopropyloxazolo[5,4-b]pyridin-5-yl)(1-methyl-3,4-dihydroisoquinolin-2(1H)-yl)methanone

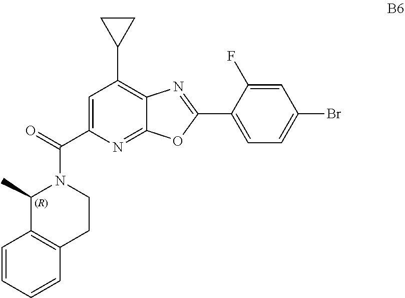

To a solution of intermediate B5 (200 mg, 0.452 mmol) in MeCN (1.5 mL) was added isoamylnitrite (91 μL, 0.68 mmol) dropwise then warmed at 35° C. and stirred for 20 minutes. The reaction mixture was then allowed to cool down to room temperature and purged with N₂. CuBr₂ (76 mg, 0.34 mmol) was added in one portion. The reaction mixture was purged again with N₂, warmed to 35° C. and stirred for 1 hour. Extra CuBr₂ (15 mg, 0.068 mmol) was added, the reaction mixture was purged again with nitrogen, warmed to 35° C. and stirred for 1 hour. The mixture was cooled down to rt then water and EtOAc were added and the layers were separated. The aqueous layer was extracted with EtOAc (once). The combined organic layers were dried over MgSO₄, filtered and the solvent was removed in vacuo. The residue was purified by preparative LC (regular SiOH 40 μm, 24 g Buchi, mobile phase gradient: from heptane/EtOAc from 90/10 to 30/70). The fractions containing product were combined and evaporated under vacuum to give intermediate B6 (161 mg, 70%).

### Compound 2

### (7-cyclopropyl-2-(4-((3 S,4S)-3,4-dihydroxypyrrolidin-1-yl)-2-fluorophenyl)oxazolo[5,4-b]pyridin-5-yl)((R)-1-methyl-3,4-dihydroisoquinolin-2(1H)-yl)methanone

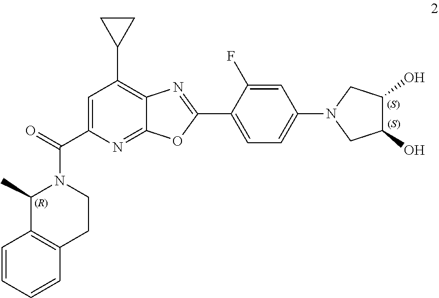

In a sealed tube, a mixture of intermediate B6 (128 mg, 253 μmol), (3S,4S)-Pyrrolidine-3,4-diol (26 mg, 0.25 mmol) and K₂CO₃ (122 mg, 0.885 mmol) in THE (3.0 mL) was degassed with N₂ for 10 min. DavePhos (20 mg, 51 μmol) and Pd₂(dba)₃ (23 mg, 25 μmol) were added and the mixture was purged with N₂. The mixture was heated at 75° C. for 18 h. The mixture was cooled down to rt then EtOAc and water were added and the layers were separated. The aqueous layer was extracted with EtOAc. The combined organic layers were washed with brine, dried over MgSO₄ and concentrated. The residue was purified by preparative LC (regular SiOH 40 μm, 24 g Buchi®, mobile phase gradient: from DCM/PrOH 99/1 to 84/16). The fractions containing product were combined and evaporated under vacuum. The residue was purified by preparative LC (spherical C18 25 μm, 40 g YMC-ODS-25, dry loading (Celite®), mobile phase gradient 0.2% aq. NH₄⁺HCO₃⁻/MeCN from 70:30 to 30:70). The fractions containing product were combined and freeze dried to give compound 2 (56 mg, 42%) as a white solid.

### Synthesis of Benzofurane

### 1.3 Synthesis of Compound 3

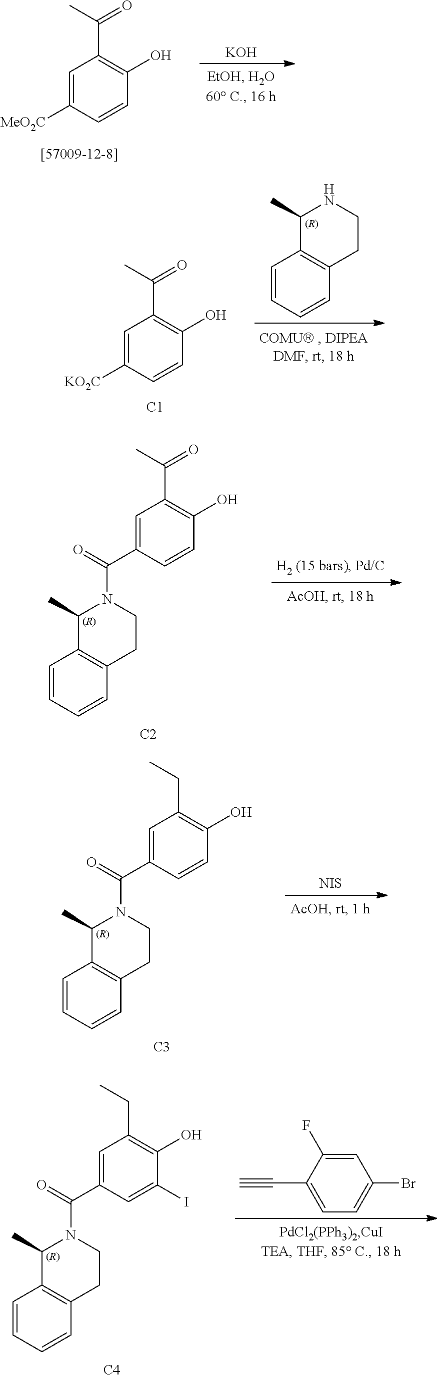

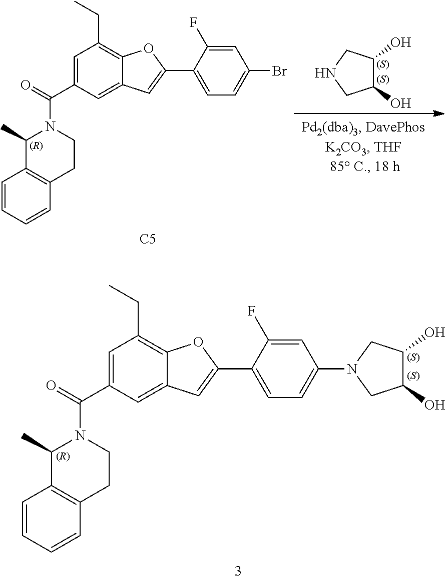

### Intermediate C1

### Potassium 3-acetyl-4-hydroxybenzoate

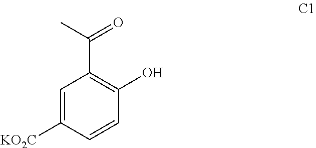

A mixture of methyl 3-acetyl-4-hydroxybenzoate (1.68 g, 8.65 mmol) and potassium hydroxide (933 mg, 16.6 mmol) in EtOH (31 mL) and H₂O (1 mL) was stirred at 60° C. for 16 h. The mixture was cooled down to rt then the solid was filtered and dried over frit to give intermediate C1 (1.9 g, quant.).

### Intermediate C2

### (R)-1-(2-hydroxy-5-(1-methyl-1,2,3,4-tetrahydroisoquinoline-2-carbonyl)phenyl)ethan-1-one

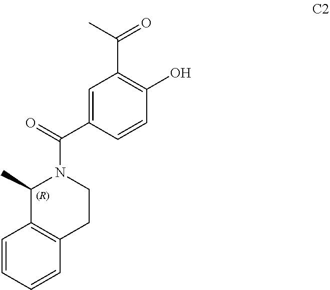

A mixture of intermediate C1 (1.8 g, 8.2 mmol), (1R)-methyl-(1,2,3,4)-tetrahydroisoquinoline (1.46 g, 9.90 mmol), COMU® (8.83 g, 20.6 mmol) and DIPEA (4.4 mL, 26 mmol) in DMF (48 mL) was stirred at rt for 18 h. The reaction mixture was diluted in ethyl acetate, washed with a sat. aq. solution of NaHCO₃, brine, dried over MgSO₄ and evaporated in vacuo to give a residue which was purified by preparative LC (regular SiOH 40 μm, 40 g Buchi®, mobile phase gradient: from heptane/EtOAc 90:10 to 70:30). The fractions containing product were combined and evaporated under vacuum to give intermediate C2 as a yellow oil (187 mg, 7%).

### Intermediate C3

### (R)-(3-ethyl-4-hydroxyphenyl)(1-methyl-3,4-dihydroisoquinolin-2(1H)-yl)methanone

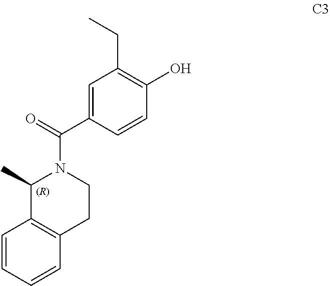

In an autoclave intermediate C2 (187 mg, 0.604 mmol) and acetic acid (1.1 mL) was stirred at rt under H₂ atmosphere (15 bars) with palladium on active charcoal, wet (5%) (386 mg, 0.181 mmol) as catalyst for 18 h. The mixture was filtered over Celite® and evaporated to dryness. The residue was purified by preparative LC (regular SiOH, 40 μm, Buchi® 24 g, mobile phase gradient: from Heptane/EtOAc 90:10 to 20:80). The fractions containing product were combined and evaporated under vacuum to give intermediate C3 (122 mg, 68%).

### Intermediate C4

### (R)-(3-ethyl-4-hydroxy-5-iodophenyl)(1-methyl-3,4-dihydroisoquinolin-2(1H)-yl)methanone

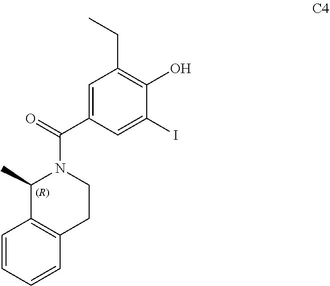

NIS (85 mg, 0.38 mmol) was added portionwise to a stirred suspension of intermediate C3 (102 mg, 0.345 mmol) in acetic acid (1.0 mL) at rt. The mixture was stirred at rt for 1 h. The solvent was removed under vacuum then the residue was purified by preparative LC (regular SiOH, 40 μm Buchi® 12 g, mobile phase gradient: from Heptane/AcOEt 90:10 to 20:80). The fractions containing product were combined and evaporated under vacuum to give intermediate C4 (103 mg, 71%).

### Intermediate C5

### (R)-(2-(4-bromo-2-fluorophenyl)-7-ethylbenzofuran-5-yl)(1-methyl-3,4-dihydroisoquinolin-2(1H)-yl)methanone

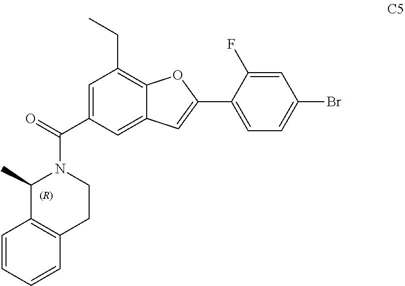

A mixture of intermediate C4 (122 mg, 0.290 mmol), 4-bromo-1-ethynyl-2-fluorobenzene (75 mg, 0.38 mmol), TEA (121 μL, 0.869 mmol) and THE (2.4 mL) was purged with N₂. PdCl₂(PPh₃)₂ (61 mg, 0.087 mmol) and Cut (55 mg, 0.29 mmol) were added and the mixture was purged with N₂. The mixture was heated at 85° C. for 18 h. The mixture was cooled down to rt then water and EtOAc were added and an extraction was performed. The aqueous layer was extracted with EtOAc. The organic layers were combined, washed with brine, dried (MgSO₄), filtered, evaporated, and purified by preparative LC (regular SiOH, 40 μm, Buchi® 24 g, mobile phase gradient: from Heptane/EtOAc 90:10 to 40:60) The fractions containing product were combined and evaporated under vacuum to give intermediate C5 (81 mg, 57%).

### Compound 3

### (2-(4-((3S,4S)-3,4-dihydroxypyrrolidin-1-yl)-2-fluorophenyl)-7-ethylbenzofuran-5-yl)((R)-1-methyl-3,4-dihydroisoquinolin-2 (1H)-yl)methanone

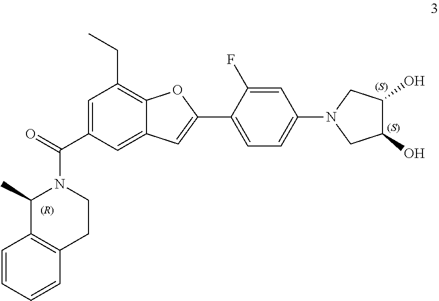

In a sealed tube, a mixture of intermediate C5 (81.0 mg, 165 μmol), (3S,4S)-pyrrolidine-3,4-diol (17 mg, 0.17 mmol) and K₂CO₃ (80 mg, 0.58 mmol) in THF (1.9 mL) was degassed with N₂ for 10 min. DavePhos (13 mg, 33 μmol) and Pd₂(dba)₃ (15 mg, 16 μmol) were added and the mixture was purged with N₂. The mixture was heated at 85° C. for 18 h. The mixture was cooled down to rt then EtOAc and water were added. The aqueous layer was extracted with EtOAc. The combined organic layers were washed with brine, dried over MgSO₄ and concentrated. The residue was purified by preparative LC (regular SiOH, 40 μm, 24 g Buchi, mobile phase gradient: from Heptane/EtOAc 50:50 to à:100). The fractions containing product were combined and evaporated under vacuum to give a brownish solid which was purified by preparative LC (spherical C18 25 μm, 40 g YMC-ODS-25, dry loading (Celite), mobile phase gradient 0.2% aq. NH₄⁺HCO₃⁻/MeCN from 65:35 to 25:75). The fractions containing product were combined and freeze dried to give compound 3 (35 mg, 41%) as a white solid.

### 2. Synthesis of Thienopyridine

### 2.1 Synthesis of Compound 4

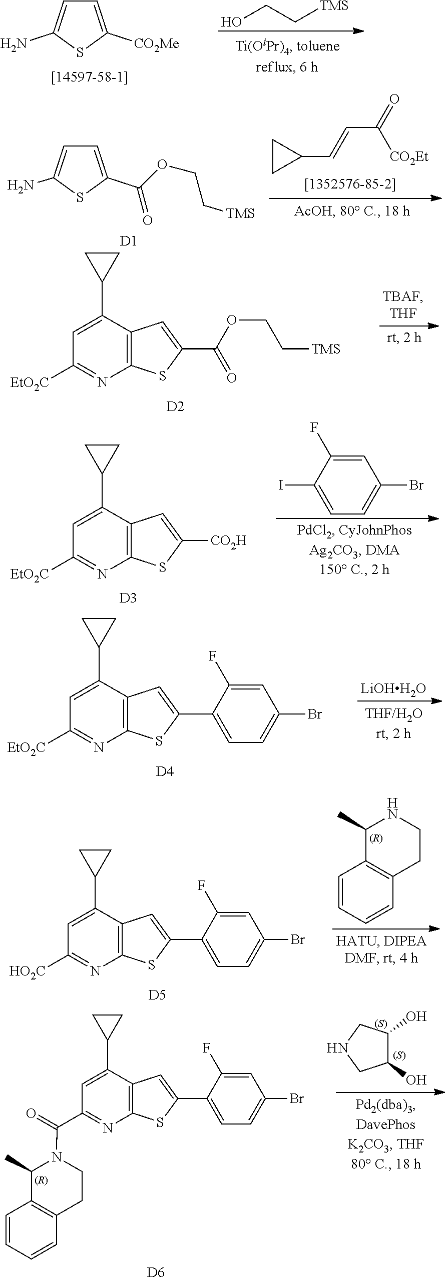

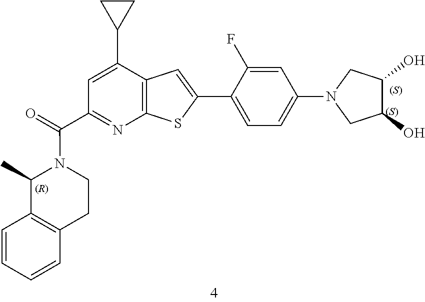

### Intermediate D1

### 2-(trimethylsilyl)ethyl 5-aminothiophene-2-carboxylate

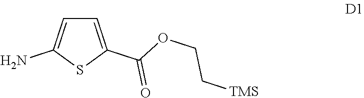 [SMILES: C[Si](C)(C)CCOC(=O)c1ccc(N)s1]

A mixture of 5-aminothiophene-2-carboxylic acid methyl ester (1.58 g, 10.1 mmol), 2-trimethyl-silylethanol (7.2 mL, 50.3 mmol), Ti(OⁱPr)₄ (3 mL, 10.1 mmol) and toluene (36 ml) was stirred at reflux for 6 h with a dean-stark trap with MS 5 Å. The mixture was cooled down to rt, evaporated then purified by preparative LC (irregular SiOH 15-40 μm, 80 g GraceResolv®, mobile phase gradient: from Heptane/EtOAc 100:0 to 50:50). The fractions containing product were combined and evaporated under vacuum to give intermediate D1 as a black oil (1.26 g at 83% purity, 43%).

### Intermediate D2

### 6-ethyl 2-(2-(trimethylsilyl)ethyl) 4-cyclopropylthieno[2,3-b]pyridine-2,6-dicarboxylate

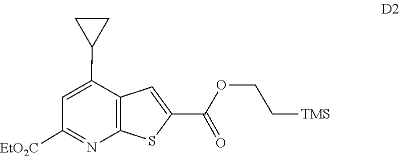 [SMILES: CCOC(=O)c1cc(C2CC2)c2cc(C(=O)OCC[Si](C)(C)C)sc2n1]

A mixture of intermediate D1 (1.26 g, 4.30 mmol, 83% purity), (3E)-4-cyclopropyl-2-oxo-3-butenoic acid ethyl ester (705 mg, 4.96 mmol) and acetic acid (8 mL) was stirred at 80° C. for 18 h. The mixture was cooled down to rt then the solid was filtered. The filtrate was evaporated and purified by preparative LC (spherical C18 25 μm, 40 g YMC-ODS-25, mobile phase gradient 0.2% aq. NH₄⁺HCO₃⁻/MeCN from 50:50 to 0:100). The fractions containing product were combined and evaporated under vacuum to give intermediate D2 (449 mg, 27%).

### Intermediate D3

### 4-cyclopropyl-6-(ethoxycarbonyl)thieno[2,3-b]pyridine-2-carboxylic acid

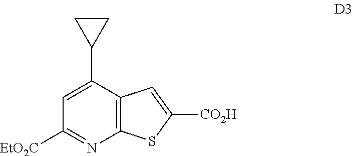 [SMILES: CCOC(=O)c1cc(C2CC2)c2cc(C(=O)O)sc2n1]

A mixture of intermediate D2 (449 mg, 1.15 mmol), TBAF 1M in THF (1.17 mL, 1.17 mmol) and THF (3 mL) was stirred at rt for 2 h. 10% aq. KHSO₄ and EtOAc were added. The layers were separated and the aqueous layer was extracted with EtOAc. The combined organic layers were dried over MgSO₄, filtered and evaporated to give intermediate D3 (390 mg at 85% purity, quant.).

### Intermediate D4

### Ethyl 2-(4-bromo-2-fluorophenyl)-4-cyclopropylthieno[2,3-b]pyridine-6-carboxylate

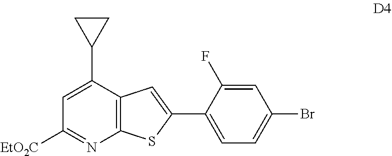 [SMILES: CCOC(=O)c1cc(C2CC2)c2cc(-c3ccc(Br)cc3F)sc2n1]

A mixture of intermediate D3 (390 mg, 1.14 mmol, purity 85%), 1-bromo-3-fluoro-4-iodobenzene (342 mg, 1.14 mmol), Ag₂CO₃ (941 mg, 3.41 mmol) in DMA (15 mL) was purged with N₂. PdCl₂ (20 mg, 0.114 mmol) and CyJohnPhos (80 mg, 0.228 mmol) were added. The mixture was purged with N₂ then stirred at 150° C. for 2 h. The mixture was cooled down to rt then water and EtOAc were added and the layers were separated. The aqueous layer was extracted with EtOAc. The combined organic layers were washed with brine, dried over MgSO₄, filtered, evaporated and purified by preparative LC (irregular SiOH 15-40 μm, 24 g GraceResolv®, mobile phase gradient: from Heptane/EtOAc 100:0 to 0:100). The fractions containing product were combined and evaporated under vacuum then the residue was purified again by reverse phase (spherical C18 25 μm, 40 g YMC-ODS-25, mobile phase gradient 0.2% aq. NH₄⁺HCO₃⁻/MeCN from 50:50 to 0:100). The fractions containing product were combined and evaporated under vacuum to give intermediate D4 (46 mg, 10%).

### Intermediate D5

### 2-(4-bromo-2-fluorophenyl)-4-cyclopropylthieno[2,3-b]pyridine-6-carboxylic acid

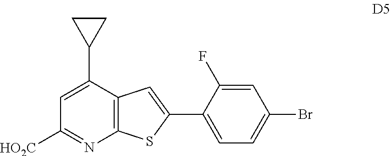 [SMILES: O=C(O)c1cc(C2CC2)c2cc(-c3ccc(Br)cc3F)sc2n1]

A mixture of intermediate D4 (76 mg, 0.181 mmol) and lithium hydroxide monohydrate (15 mg, 0.362 mmol) in THE (4.1 mL) and H₂O (0.33 mL) was stirred at room temperature for 2 h. EtOAc and 10% aq. KHSO₄ were added to the mixture. The layers were separated and the aqueous layer was extracted with EtOAc. The organic layer was combined, washed with brine, dried over MgSO₄, filtered and evaporated to give of intermediate D5 as yellow solid (67 mg, Quant.).

### Intermediate D6

### (R)-(2-(4-bromo-2-fluorophenyl)-4-cyclopropylthieno[2,3-b]pyridin-6-yl)(1-methyl-3,4-dihydroisoquinolin-2(1H)-yl)methanone

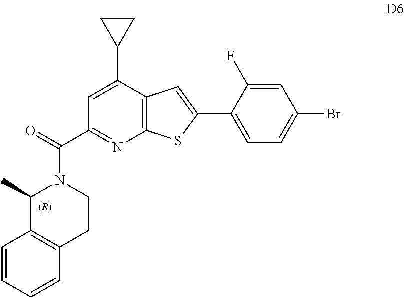

A mixture of intermediate D5 (67 mg, 0.171 mmol), (1R)-methyl-(1,2,3,4)-tetrahydroisoquinoline (28 mg, 0.188 mmol), HATU (97 mg, 0.256 mmol) and DIPEA (90 μL, 0.512 mmol) in DMF (1.2 mL) was stirred at rt for 4 h. Water and EtOAc were added to the reaction mixture. The layers were separated. The aqueous layer was extracted twice with EtOAc. The combined organic layers were washed with brine (3 times), dried over MgSO₄, filtered, evaporated and purified by preparative LC (irregular SiOH 15-40 μm, 40 g GraceResolv®, mobile phase gradient: from Heptane/EtOAc 75:25 to 0:100). The fractions containing product were combined and evaporated under vacuum to give intermediate D6 as a white solid (86 mg, 97%).

### Compound 4

### (4-cyclopropyl-2-(4-((3 S,4S)-3,4-dihydroxypyrrolidin-1-yl)-2-fluorophenyl)thieno[2,3-b]pyridin-6-yl)((R)-1-methyl-3,4-dihydroisoquinolin-2(1H)-yl)methanone

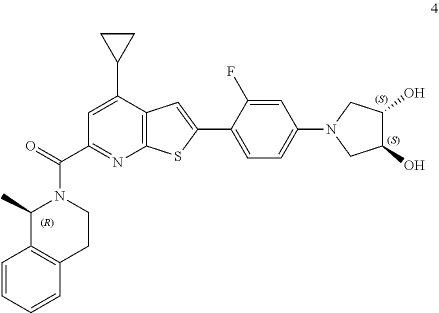

Under N₂, a mixture of intermediate D6 (76 mg, 0.146 mmol), (3S,4S)-Pyrrolidine-3,4-diol (18 mg, 0.175 mmol) and K₂CO₃ (60 mg, 0.437 mmol) in THF (1.4 mL) was degassed with N₂ for 10 min. DavePhos (23 mg, 0.0583 mmol) and Pd₂(dba)₃ (13 mg, 0.0146 mmol) were added and the reaction mixture was purged with N₂. The mixture was heated at 80° C. for 18 h. The mixture was cooled down to rt then water and EtOAc were added. The aqueous layer was extracted with EtOAc, the combined organic layers were dried over MgSO₄, filtered, concentrated in vacuo and purified by preparative LC (regular SiOH 40 μm, 40 g Buchi®, mobile phase gradient: from DCM/MeOH 100:0 to 90:10). The fractions containing product were combined and evaporated under vacuum. The residue was solubilized with MeCN/water and freeze-dried to give compound 4 as a yellow solid (57 mg, 72%).

### 3. Synthesis of thiazolopyridine

### 3.1 Synthesis of Compound 5

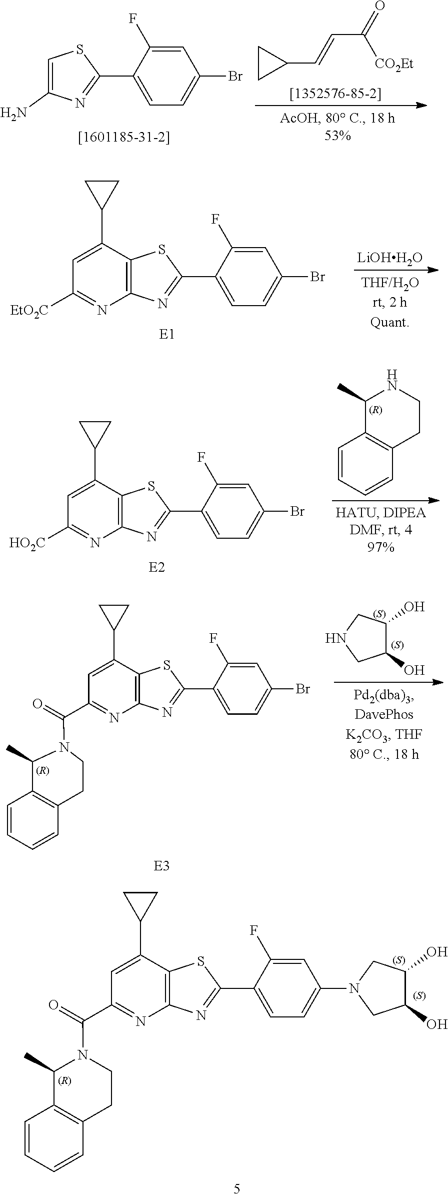

### Intermediate E1

### Ethyl 2-(4-bromo-2-fluorophenyl)-7-cyclopropylthiazolo[4,5-b]pyridine-5-carboxylate

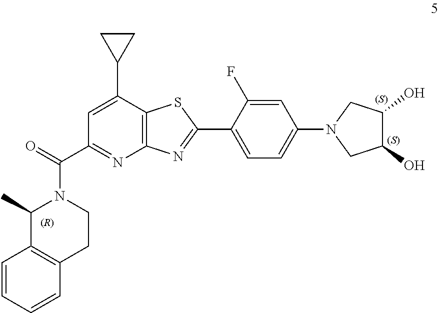

A mixture of 2-(4-bromo-2-fluorophenyl)-4-thiazolamine (174 mg, 0.637 mmol), (3E)-4-cyclopropyl-2-oxo-3-butenoic acid ethyl ester (107 mg, 0.637 mmol) and acetic acid (2.5 mL) was stirred at 80° C. for 18 h. The mixture was cooled down to rt, evaporated then purified by preparative LC (irregular SiOH 15-40 μm, 40 g GraceResolv®, mobile phase gradient: from heptane/EtOAc 100:0 to 50:50). The fractions containing product were combined and evaporated under vacuum to give intermediate E1 (142 mg, 53%).

### Intermediate E2

### 2-(4-bromo-2-fluorophenyl)-7-cyclopropylthiazolo[4,5-b]pyridine-5-carboxylic acid

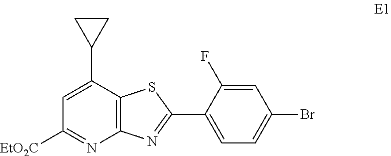 [SMILES: CCOC(=O)c1cc(C2CC2)c2sc(-c3ccc(Br)cc3F)nc2n1]

A mixture of intermediate E1 (142 mg, 0.337 mmol) and lithium hydroxide monohydrate (28 mg, 0.674 mmol) in THE (7.6 mL) and H₂O (0.6 mL) was stirred at room temperature for 2 h. EtOAc and 10% aq. KHSO₄ were added to the mixture. The layers were separated and the aqueous layer was extracted with EtOAc (twice). The combined organic layer was washed with brine, dried over MgSO₄, filtered and evaporated to give intermediate E2 as yellow solid (116 mg, 88%).

### Intermediate E3

### (R)-(2-(4-bromo-2-fluorophenyl)-7-cyclopropylthiazolo[4,5-b]pyridin-5-yl)(1-methyl-3,4-dihydroisoquinolin-2(1H)-yl)methanone

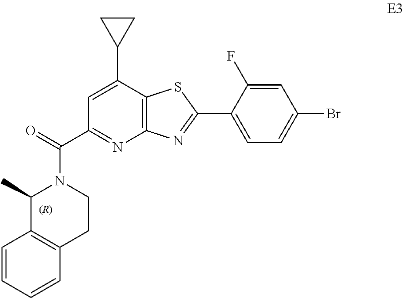

A mixture of intermediate E2 (116 mg, 0.295 mmol), (1R)-methyl-(1,2,3,4)-tetrahydroisoquinoline (48 mg, 0.324 mmol), HATU (168 mg, 0.442 mmol) and DIPEA (155 μL, 0.885 mmol) in DMF (2 mL) was stirred at rt for 18 h. Water and EtOAc were added to the reaction mixture. The layers were separated. The aqueous layer was extracted twice with EtOAc. The combined organic layers were washed with brine (3 times), dried over MgSO₄, filtered, evaporated and purified by preparative LC (irregular SiOH 15-40 μm, 40 g GraceResolv®, mobile phase gradient: from Heptane/EtOAc 75:25 to 0:100). The fractions containing product were combined and evaporated under vacuum to give intermediate E3 as a yellow solid (100 mg, 65%).

### Compound 5

### (7-cyclopropyl-2-(4-((3 S,4S)-3,4-dihydroxypyrrolidin-1-yl)-2-fluorophenyl)thiazolo[4,5-b]pyridin-5-yl)((R)-1-methyl-3,4-dihydroisoquinolin-2(1H)-yl)methanone

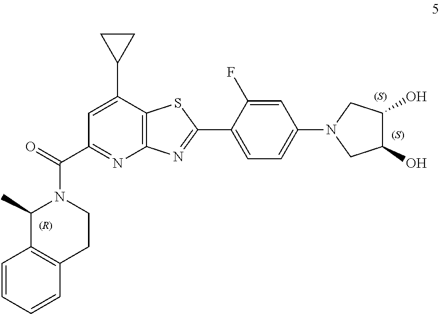

Under N₂, a mixture of intermediate E3 (90 mg, 0.172 mmol), (3S,4S)-pyrrolidine-3,4-diol (21 mg, 0.207 mmol) and K₂CO₃ (71 mg, 0.517 mmol) in THE (1.6 mL) was degassed with N₂. DavePhos (27 mg, 0.0689 mmol) and Pd₂(dba)₃ (16 mg, 0.0172 mmol) were added and the reaction mixture was purged with N₂. The mixture was heated at 80° C. for 18 h. The reaction was cooled down to rt then water and EtOAc were added. The layers were separated and the aqueous layer was extracted with EtOAc (twice). The combined organic layers were dried over MgSO₄, filtered, concentrated in vacuo and purified by preparative LC (regular SiOH 40 μm, 40 g Buchi®, mobile phase gradient: from DCM/MeOH 100:0 to 88:12). The fractions containing product were evaporated under vacuum then solubilized with MeCN/water and freeze-dried to give compound 5 as a yellow solid (21 mg, 22%).

### 4. Synthesis of furopyridine

### 4.1 Synthesis of Compound 6

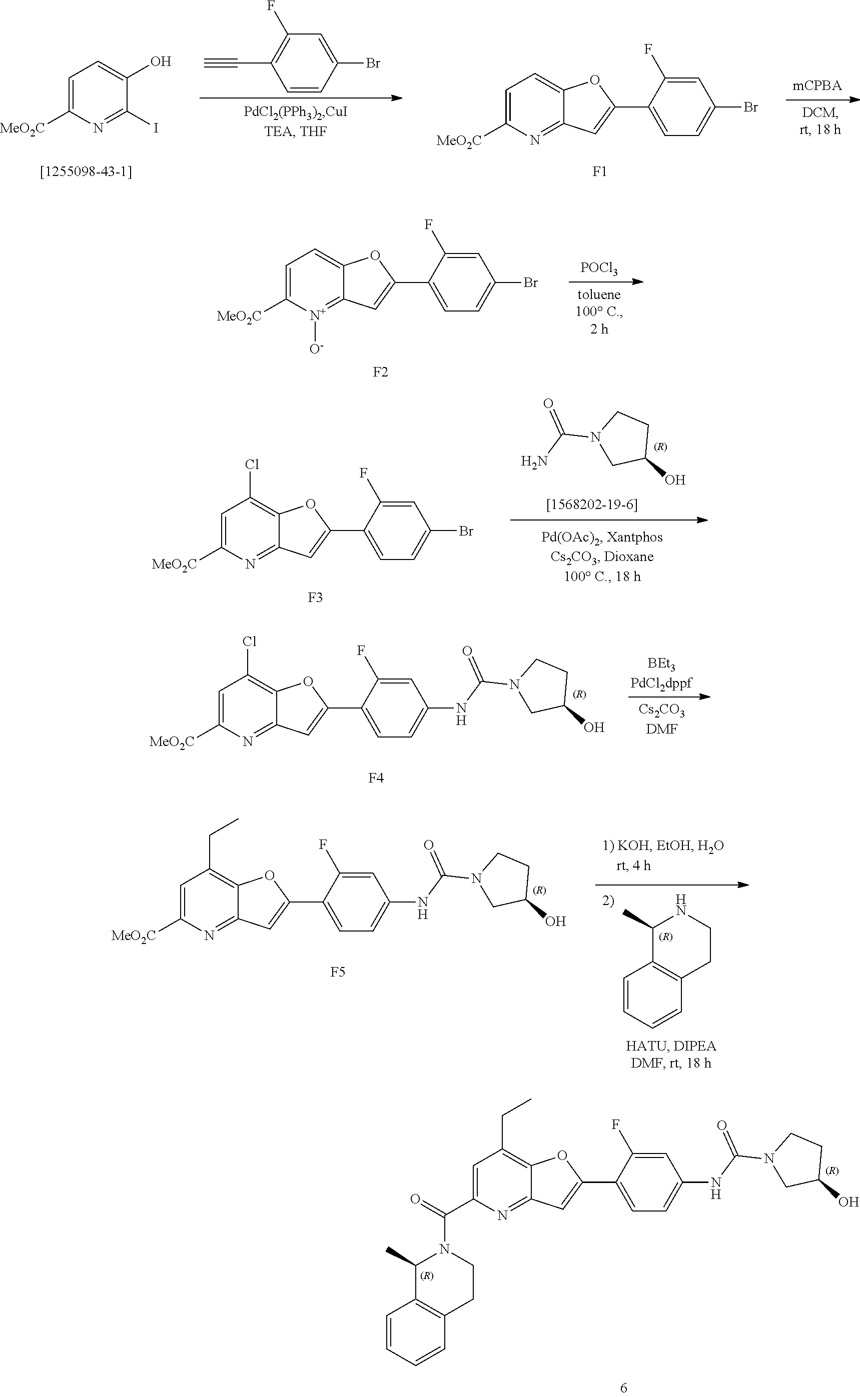

### Intermediate F1

### Methyl 2-(4-bromo-2-fluorophenyl)furo[3,2-b]pyridine-5-carboxylate

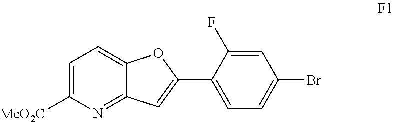 [SMILES: COC(=O)c1ccc2oc(-c3ccc(Br)cc3F)cc2n1]

A mixture of methyl 5-hydroxy-6-iodopyridine-2-carboxylate (4.8 g, 15.5 mmol), 4-bromo-1-ethynyl-2-fluorobenzene (4.0 g, 20 mmol), TEA (4.8 mL, 34.6 mmol) and THE (28 mL) was purged with N₂. PdCl₂(PPh₃)₂ (1.2 g, 1.7 mmol) and Cut (680 mg, 3.58 mmol) were added and the mixture was purged with N₂. The mixture was heated at 100° C. using one single mode microwave (Biotage Initiator EXP 60) with a power output ranging from 0 to 400 W for 30 min [fixed hold time]. The mixture was cooled down to rt then water and EtOAc were added. The layers were separated ant the aqueous layer was extracted with EtAOc. The combined organic layer was washed with brine, dried over MgSO₄, filtered, evaporated and purified by preparative LC (irregular SiOH 15-40 μm, 120 g GraceResolv®, mobile phase gradient: heptane/EtOAc from 100:0 to 25:75). The fractions containing product were combined and evaporated under vacuum. The residue was purified by preparative LC (spherical C18 25 μm, 300 g YMC-ODS-25, dry loading (Celite®), mobile phase gradient 0.2% aq. NH₄⁺HCO₃⁻/MeCN from 60:40 to 0:100). The fraction containing product were concentrated then DCM and water were added. The layers were separated and the aqueous layer was extracted with DCM. The combined organic layers were washed with brine, dried over MgSO₄, filtered and evaporated to give intermediate F1 as off-white solid (2.02 g, 25%).

### Intermediate F2

### 2-(4-bromo-2-fluorophenyl)-5-(methoxycarbonyl)furo[3,2-b]pyridine 4-oxide

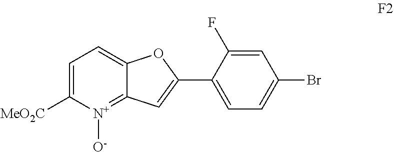 [SMILES: COC(=O)c1ccc2oc(-c3ccc(Br)cc3F)cc2[n+]1[O-]]

A mixture of intermediate F1 (2.02 g, 5.77 mmol), mCPBA (4 g, 23.2 mmol) and DCM (26 mL) was stirred at rt for 18 h. An extraction was performed with aq. NaHCO₃ and DCM, the organic layer was washed with brine, dried over MgSO₄ and evaporated to give intermediate F2 as yellow solid (2.4 g at 88% purity, quant.).

### Intermediate F3

### Methyl 2-(4-bromo-2-fluorophenyl)-7-chlorofuro[3,2-b]pyridine-5-carboxylate

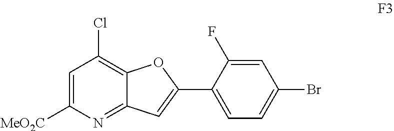 [SMILES: COC(=O)c1cc(Cl)c2oc(-c3ccc(Br)cc3F)cc2n1]

A mixture of intermediate F2 (2.4 g, 5.77 mmol, 88% purity), POCl₃ (2.7 mL, 28.8 mmol) and DCM (35 mL) was stirred at 100° C. for 2 h. The mixture was cooled down to rt then NaHCO₃ aq. was added slowly and the layers were separated. The aqueous layer was extracted with EtOAc (once). The combined organic layers were dried over MgSO₄, filtered and evaporated. The residue was purified by preparative LC (irregular SiOH 15-40 μm, 120 g GraceResolv®, mobile phase gradient: heptane/EtOAc from 100:0 to 50:50). The fractions containing product were combined and evaporated under vacuum to give intermediate F3 as white solid (1.08 g, 49%).

### Intermediate F4

### Methyl (R)-7-chloro-2-(2-fluoro-4-(3-hydroxypyrrolidine-1-carboxamido)phenyl)furo[3,2-b]pyridine-5-carboxylate

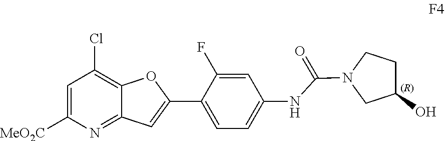

A mixture of intermediate F3 (1.08 g, 2.81 mmol), (3R)-3-hydroxy-1-pyrrolidinecarboxamide (393 mg, 2.81 mmol) and Cs₂CO₃ (2.75 g, 8.43 mmol) was charged in a sealed tube and purged with N₂. 1,4 dioxane (59 mL) was added and the mixture was degassed with N₂, then Pd(OAc)₂ (63 mg, 0.281 mmol) and Xantphos (162 mg, 0.281 mmol) were added. The reaction mixture was purged with N₂ then was stirred and heated at 100° C. for 18 h. The mixture was cooled down to rt then EtOAc and water were added and the mixture was filtered over Celite®. The layers were separated and the aqueous layer was extracted with EtOAc (once). The combined organic layers were washed with brine, dried over MgSO₄, evaporated and purified by preparative LC (irregular SiOH 15-40 μm, 80 g GraceResolv®, mobile phase gradient: DCM/MeOH from 100:0 to 95:5). The fractions containing product were combined and evaporated under vacuum to give intermediate F4 as pale yellow solid (259 mg, 21%).

### Intermediate F5

### Methyl (R)-7-ethyl-2-(2-fluoro-4-(3-hydroxypyrrolidine-1-carboxamido)phenyl)furo[3,2-b]-pyridine-5-carboxylate

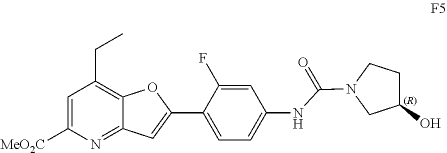

A mixture of intermediate F4 (259 mg, 597 μmol), triethylborane 1M in THE (1.8 mL, 1.79 mmol), Cs₂CO₃ (973 mg, 2.99 mmol) and DMF (10 mL) was purged with N₂. PdCl₂dppf (52 mg, 70 μmol) was added the mixture was stirred at 70° C. for 18 h. The mixture was cooled down to rt. The solvent was evaporated then the residue was purified by preparative LC (spherical C18 25 μm, 40 g YMC-ODS-25, dry loading (Celite®), mobile phase gradient 0.2% aq. NH₄⁺HCO₃⁻/MeCN from 95:05 to 50:50). The fractions containing product were freeze-dried to give F5 (100 mg, 39%) as white solid.

### Compound 6

### (R)-N-(4-(7-ethyl-5-((R)-1-methyl-1,2,3,4-tetrahydroisoquinoline-2-carbonyl)furo[3,2-b]pyridin-2-yl)-3-fluorophenyl)-3-hydroxypyrrolidine-1-carboxamide

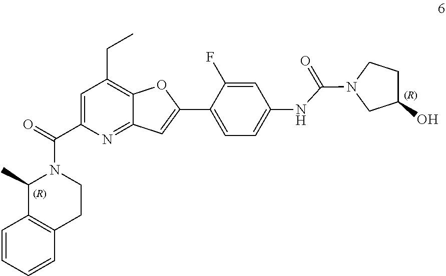

A mixture of intermediate F5 (100 mg, 0.234 mmol) and potassium hydroxide (26 mg, 0.468 mmol) in EtOH (2 mL) and H₂O (162 μL) was stirred at rt for 4 h. The mixture was evaporated under vacuum and coevaporated (3 times) with THF. The residue was solubilized in DMF (2 mL) then (1R)-methyl-(1,2,3,4)-tetrahydroisoquinoline (41 mg, 0.279 mmol), HATU (133 mg, 0.349 mmol) and DIPEA (122 μL, 0.698 mmol) were added. The resulting mixture was stirred at rt for 18 h. EtOAc and water were added. The organic layer was separated, washed with brine, dried over MgSO₄, filtered and purified by preparative LC (irregular SiOH 15-40 μm, 24 g GraceResolv®, mobile phase gradient: DCM/MeOH from 100:0 to 90:10). The fractions containing product were combined and evaporated under vacuum. The residue was purified by preparative LC (spherical C18 25 μm, 40 g YMC-ODS-25, mobile phase gradient 0.2% aq. NH₄⁺HCO₃⁻/MeCN from 90:10 to 10:90). The fractions containing product were freeze-dried to give compound 6 (55 mg, 44%) as white solid.

### 5. Synthesis of Azaindazole

### 5.1 Synthesis of Compound 7

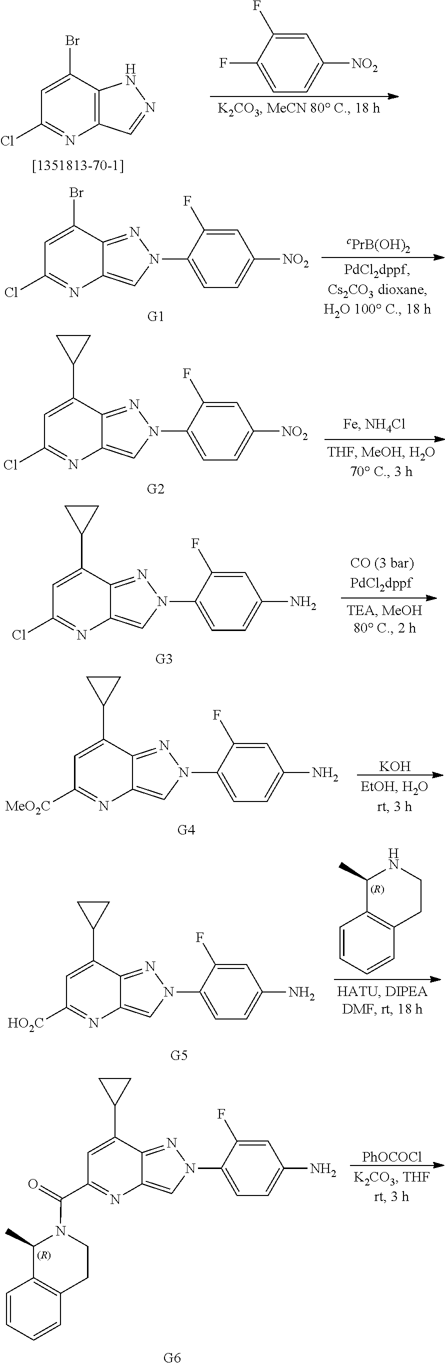

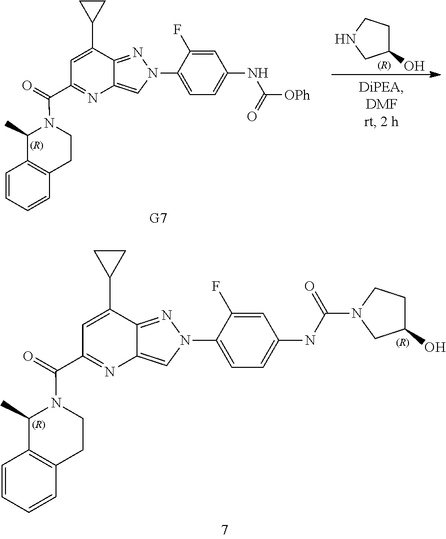

### Intermediate G1

### 7-bromo-5-chloro-2-(2-fluoro-4-nitrophenyl)-2H-pyrazolo[4,3-b]pyridine

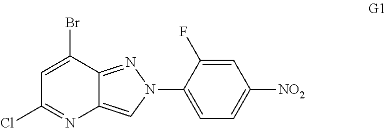 [SMILES: O=[N+]([O-])c1ccc(-n2cc3nc(Cl)cc(Br)c3n2)c(F)c1]

7-Bromo-5-chloro-1H-pyrazolo[4,3-b]pyridine (1.12 g, 4.72 mmol), 3,4-difluoronitrobenzene (575 μL, 5.19 mmol), K₂CO₃ (1.96 g, 14.2 mmol) in MeCN (36 mL) was stirred stirred at 80° C. for 18 h. The mixture was cooled down to rt then water and EtOAc were added. The layers were separated and the aqueous layer was extracted with EtOAc. The combined organic layers were dried over MgSO₄, filtered and concentrated in vacuo. The residue was purified by preparative LC (irregular SiOH 15-40 μm, 40 g GraceResolv®, dry loading (Celite®), mobile phase gradient: from Heptane/EtOAc 100:0 to 50:50 and wash with DCM/MeOH 100:00 to 90:10). The fractions containing product were combined and evaporated under vacuum to give intermediate G1 (1.1 g, 63%).

### Intermediate G2

### 5-chloro-7-cyclopropyl-2-(2-fluoro-4-nitrophenyl)-2H-pyrazolo[4,3-b]pyridine

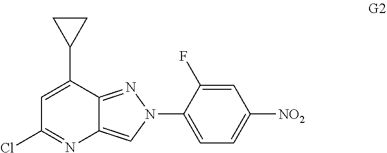 [SMILES: O=[N+]([O-])c1ccc(-n2cc3nc(Cl)cc(C4CC4)c3n2)c(F)c1]

To a degassed mixture of intermediate G1 (1.1 g, 2.9 mmol), ^{c}PrB(OH)₂ (349 mg, 4.07 mmol) and Cs₂CO₃ (4.0 g, 12 mmol) in dioxane (8.7 mL) and H₂O (87 mL) was added PdCl₂dppf (303 mg, 0.414 mmol), and the resulting mixture was stirred at 100° C. for 18 h. The mixture was cooled down to rt then EtOAc and water were added. The layers were separated and the aqueous layers was extracted with EtOAc. The combined organic layers were dried over MgSO₄, filtered and the solvent was removed in vacuo. The residue was purified by preparative LC (irregular SiOH 15-40 μm, 80 g GraceResolv®, dry loading (Celite®), mobile phase gradient: from Heptane/EtOAc 100:0 to 50:50). The fractions containing product were combined and evaporated under vacuum to give intermediate G2 (520 mg, 53%).

### Intermediate G3

### 4-(5-chloro-7-cyclopropyl-2H-pyrazolo[4,3-b]pyridin-2-yl)-3-fluoroaniline

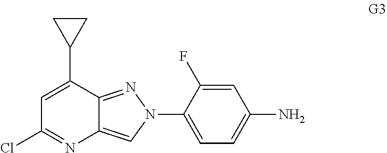 [SMILES: Nc1ccc(-n2cc3nc(Cl)cc(C4CC4)c3n2)c(F)c1]

A mixture of intermediate G2 (590 mg, 1.77 mmol), iron (495 mg, 8.87 mmol) and ammonium chloride (950 mg, 17.8 mmol) in THE (5.6 mL), MeOH (5.6 mL) and H₂O (2.8 mL) was stirred at 70° C. for 3 h. The mixture was cooled down to rt and filtered through a pad of Celite®. DCM and water were added, the layers were separated and the organic layer was dried over MgSO₄, filtered and concentrated in vacuo to give intermediate G3 (520 mg, 96%).

### Intermediate G4

### Methyl 2-(4-amino-2-fluorophenyl)-7-cyclopropyl-2H-pyrazolo[4,3-b]pyridine-5-carboxylate

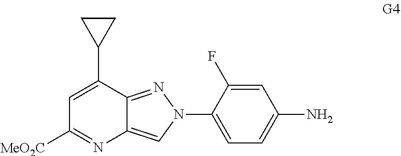 [SMILES: COC(=O)c1cc(C2CC2)c2nn(-c3ccc(N)cc3F)cc2n1]

To a degassed mixture of intermediate G3 (520 mg, 1.72 mmol) and TEA (597 μL, 4.29 mmol) in MeOH (11.8 mL) was added PdCl₂dppf (88 mg, 0.12 mmol). The resulting mixture was stirred under 3 bars of CO for 2 h at 80° C. The mixture was cooled down to rt and evaporated to dryness. DCM and water were added, the layers were separated and the organic layer was dried over MgSO₄, filtered and concentrated in vacuo. The residue was purified by preparative LC (irregular SiOH 15-40 μm, 25 g GraceResolv®, dry loading (Celite®), mobile phase gradient: from DCM/MeOH 100:0 to 98:2). The fractions containing product were combined and evaporated under vacuum to give intermediate G4 (420 mg, 75%).

### Intermediate G5

### 2-(4-amino-2-fluorophenyl)-7-cyclopropyl-2H-pyrazolo[4,3-b]pyridine-5-carboxylic acid

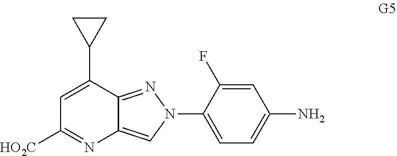 [SMILES: Nc1ccc(-n2cc3nc(C(=O)O)cc(C4CC4)c3n2)c(F)c1]

A mixture of intermediate G4 (420 mg, 1.29 mmol) and potassium hydroxide (144 mg, 2.57 mmol) in EtOH (19 mL) and H₂O (1.9 mL) was stirred at rt for 3 h. An aqueous solution of KHSO₄ 10% was added until pH=1 and the aqueous layer was extracted with EtOAc. The combined organic layers were dried over MgSO₄, filtered and concentrated in vacuo to give intermediate G5 (390 mg, 95%).

### Intermediate G6

### (R)-(2-(4-amino-2-fluorophenyl)-7-cyclopropyl-2H-pyrazolo[4,3-b]pyridin-5-yl)(1-methyl-3,4-dihydroisoquinolin-2(1H)-yl)methanone

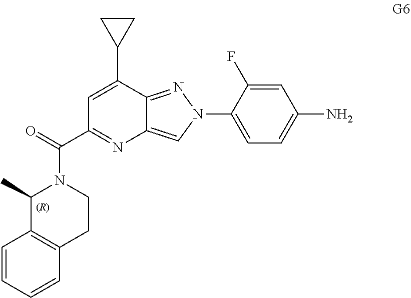

A mixture of intermediate G5 (96 mg, 0.29 mmol), (1R)-methyl-(1,2,3,4)-tetrahydroisoquinoline (86 mg, 0.58 mmol), HATU (167 mg, 0.438 mmol) and DIPEA (153 μL, 0.876 mmol) in DMF (2.0 mL) was stirred at rt for 20 h. Brine and EtOAc were added to the reaction mixture. The layers were separated. The aqueous layer was extracted twice with EtOAc. The combined organic layers were washed with brine (3 times), dried over MgSO₄, filtered and evaporated. The residue was purified by preparative LC (irregular SiOH 15-40 μm, 12 g GraceResolv®, dry loading (Celite®), mobile phase gradient: from DCM/MeOH 100:0 to 98:2) The fractions containing product were combined and evaporated under vacuum. The residue was purified by preparative LC (irregular SiOH 15-40 μm, 12 g GraceResolv®, dry loading (Celite®), mobile phase gradient: from heptane/EtOAc 100/00 to 50/50). The fractions containing product were combined and evaporated under vacuum to give intermediate G6 (82 mg, 76%).

### Intermediate G7

### phenyl (R)-(4-(7-cyclopropyl-5-(1-methyl-1,2,3,4-tetrahydroisoquinoline-2-carbonyl)-2H-pyrazolo[4,3-b]pyridin-2-yl)-3-fluorophenyl)carbamate

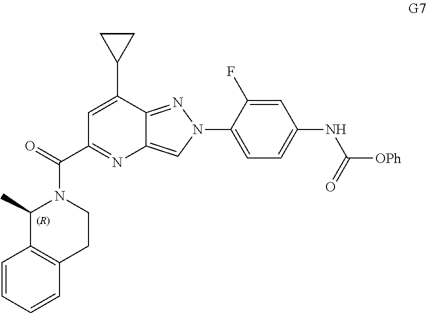

In a mixture of intermediate G6 (23 mg, 0.052 mmol) and K₂CO₃ (17 mg, 0.12 mmol) in THE (648 μL) was added phenylchloroformate (6.5 μL). The mixture was stirred at rt for 3 h. Water and EtOAc were added and the layers were separated. The aqueous layer was extracted with EtOAc. The combined organic layers were dried over MgSO₄, filtered and concentrated in vacuo to give intermediate G7 (21 mg, 72%).

### Compound 7

### (R)-N-(4-(7-cyclopropyl-5-((R)-1-methyl-1,2,3,4-tetrahydroisoquinoline-2-carbonyl)-2H-pyrazolo[4,3-b]pyridin-2-yl)-3-fluorophenyl)-3-hydroxypyrrolidine-1-carboxamide

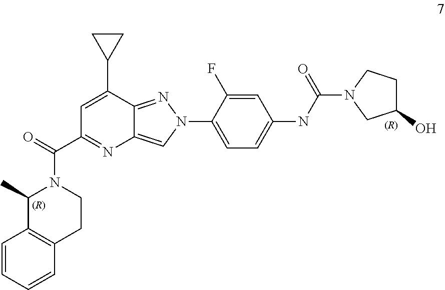

To a stirred mixture of intermediate G7 (39.5 mg, 0.0703 mmol) and (R)-(+)-3-pyrrolidinol (9.2 mg, 0.11 mmol) in DMF (1.6 mL) was added DIPEA (61 μL, 0.35 mmol). The reaction mixture was stirred at rt for 2 h. Water and EtOAc were added then the layers were separated. The aqueous layer was extracted with EtOAc. The combined organic layers were washed with brine, dried over MgSO₄, filtered and concentrated in vacuo. The residue was purified by preparative LC (irregular SiOH 15-40 μm, 4 g GraceResolv®, dry loading (Celite®), mobile phase gradient: from DCM/MeOH 100/00 to 90/10). The fractions containing product were combined and evaporated under vacuum. The residue was solubilized in MeCN (2 mL), extended with water (10 mL) and freeze-dried to give compound 7 as a white fluffy solid (19 mg, 51%).

### 6. Synthesis of Indole

### 6.1 Synthesis of Compound 8

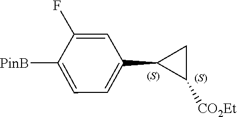

### Intermediate H0

### Ethyl (1S,2S)-2-(3-fluoro-4-(4,4,5,5-tetramethyl-1,3,2-dioxaborolan-2-yl)phenyl)cyclopropane-1-carboxylate

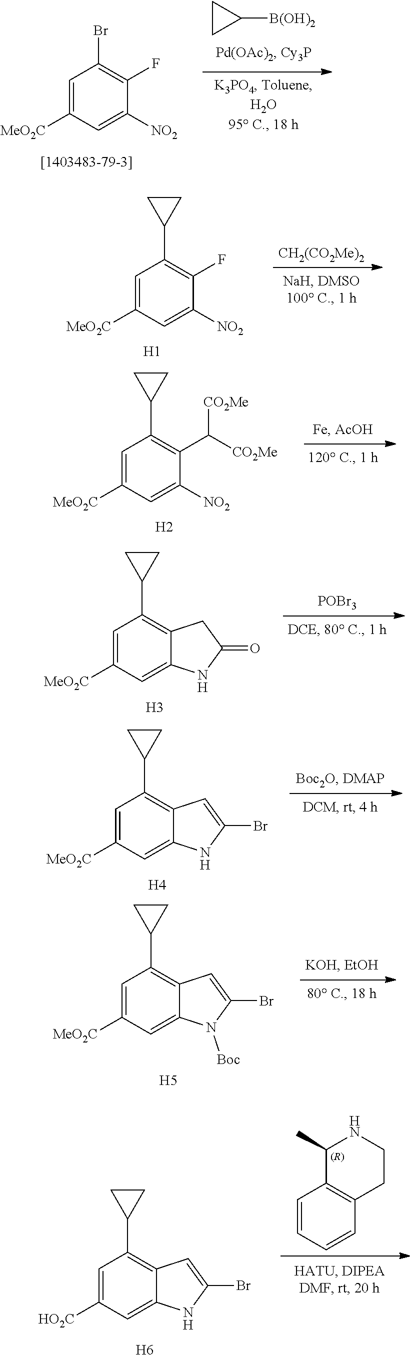

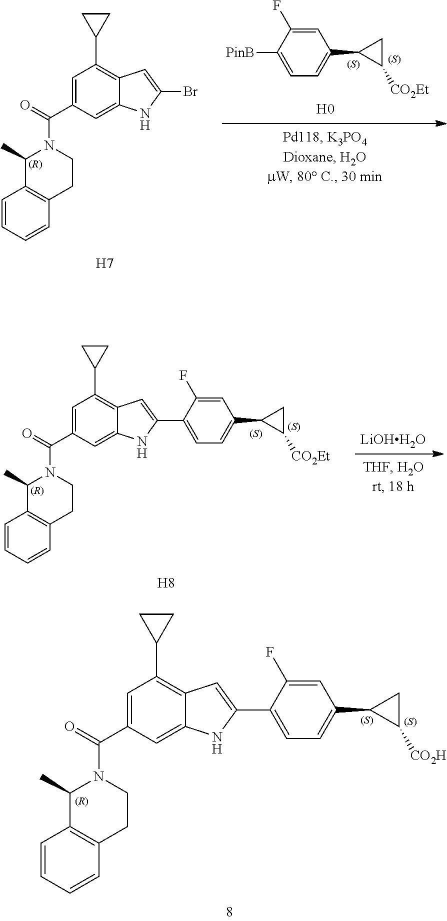

Under N₂, in a sealed tube, B₂Pin₂ (10 g, 39.4 mmol) and potassium acetate (6.8 g, 69.3 mmol) were added to a solution of (1S,2S)-2-(4-bromo-3-fluorophenyl)-cyclopropanecarboxylic acid ethyl ester (10 g, 34.8 mmol) in dioxane (170 mL). The solution was purged with nitrogen and charged with PdCl₂dppf·DCM (2.8 g, 3.42 mmol). The resulting solution was purged again with nitrogen and stirred at 100° C. for 18 h. EtOAc was added and the organic layer was washed with water and brine, dried over MgSO₄, concentrated and purified by preparative LC (irregular SiOH, 15-40 μm, Merck® 400 g, mobile phase gradient: from heptane/EtOAc 100/0 to 75/25). The fractions containing product were combined and evaporated under vacuum to give intermediate H0 as colorless oil (9.26 g, 80%).

### Intermediate H1

### Methyl 3-cyclopropyl-4-fluoro-5-nitrobenzoate

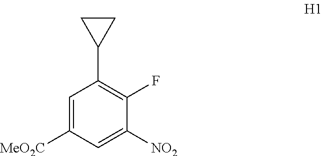 [SMILES: COC(=O)c1cc(C2CC2)c(F)c([N+](=O)[O-])c1]

To a solution of 3-bromo-4-fluoro-5-nitro-benzoic acid methyl ester (960 mg, 3.45 mmol) in toluene (20 mL), were added cyclopropylboronic acid (593 mg, 6.91 mmol), K₃PO₄ (1.83 g, 8.63 mol), tricyclohexylphoshine (290 mg, 1.04 mmol) and H₂O (4 mL). The round bottom flask was purged with N₂ (3 times) and Pd(OAc)₂ (116 mg, 518 μmol) was added. The reaction mixture was heated at 95° C. for 18 h. The mixture was cooled down to rt then EtOAc and water were added to the mixture. The layers were separated and the aqueous layer was extracted with EtOAc. The combined organic layers were washed with brine, dried over MgSO₄, filtered and concentrated. The residue was purified by preparative LC (irregular SiOH 15-40 μm, 120 g GraceResolv®, mobile phase gradient: from heptane/EtOAc from 100:0 to 85:15). The pure fractions were combined and evaporated under vacuum to give intermediate H1 as a yellow solid (460 mg, 56%).

### Intermediate H2

### Dimethyl 2-(2-cyclopropyl-4-(methoxycarbonyl)-6-nitrophenyl)malonate

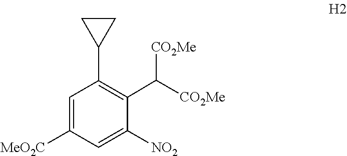 [SMILES: COC(=O)c1cc(C2CC2)c(C(C(=O)OC)C(=O)OC)c([N+](=O)[O-])c1]

A solution of NaH 60% in mineral oil (223 mg, 5.58 mmol) in DMSO (8 mL) was stirred at rt and then dimethylmalonate (352 μL, 3.08 mmol) was added dropwise. After complete addition, the reaction was stirred at 100° C. for 1 h. The mixture was cooled at rt and intermediate H1 (460 mg, 1.92 mmol) was added. The reaction was stirred at rt for 30 min then at 100° C. for 1 h. EtOAc and an aqueous saturated solution of NH₄Cl were added to the mixture. The aqueous layer was extracted with EtOAc. The organic layer was dried over MgSO₄, filtered and evaporated. The residue was purified by preparative LC (irregular SiOH 15-40 μm, 80 g GraceResolv®, mobile phase gradient: from heptane/EtOEc 100/0 to 70/30). The pure fractions were combined and evaporated under vacuum to give intermediate H2 as a white solid (470 mg, 70%).

### Intermediate H3

### Methyl 4-cyclopropyl-2-oxoindoline-6-carboxylate

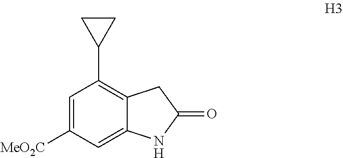 [SMILES: COC(=O)c1cc2c(c(C3CC3)c1)CC(=O)N2]

Iron (148 mg, 2.65 mmol) was added slowly to a mixture of intermediate H2 (310 mg, 882 μmol) in acetic acid (16 mL) then was stirred at 120° C. for 1 h. The reaction mixture was cooled down to rt and was filtered through a pad of Celite® and the solvent was removed in vacuo. The resulting solid was dissolved in EtOAc and water. The layers were separated and the organic layer was washed with brine, dried over MgSO₄, evaporated and purified by preparative LC (irregular SiOH, 15-40 μm, 40 g GraceResolv®, mobile phase gradient: from heptane/EtOAc 75/25 to 50/50). The pure fractions were combined and evaporated under vacuum to give intermediate H3 as a white solid (130 mg, 64%).

### Intermediate H4

### Methyl 2-bromo-4-cyclopropyl-1H-indole-6-carboxylate

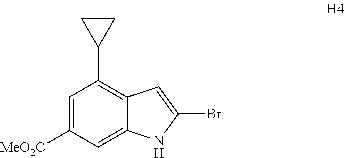 [SMILES: COC(=O)c1cc(C2CC2)c2cc(Br)[nH]c2c1]

In a solution of intermediate H3 (53 mg, 0.23 mmol) in DCE (1.5 mL) was added dropwise a solution of POBr₃ (94 mg, 0.33 mmol) in DCE (1.5 mL). The reaction mixture was stirred for 1 h at 80° C. POBr₃ (94 mg, 0.33 mmol) was added and the reaction was stirred for 2 h at 80° C. The mixture was cooled down to rt then the pH was adjusted to 7-8 by addition of an aqueous saturated NaHCO₃. The layers were separated and the organic layer was washed with brine, dried over MgSO₄, evaporated. The residue was purified by preparative LC (irregular SiOH, 15-40 μm, 24 g GraceResolv®, mobile phase gradient: from heptane/EtOAc 100/0 to 50/50). The pure fractions were combined and evaporated under vacuum to give intermediate H4 as a white solid (34 mg, 50%).

### Intermediate H5

### 1-(tert-butyl) 6-methyl 2-bromo-4-cyclopropyl-1H-indole-1,6-dicarboxylate

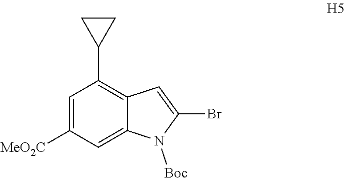 [SMILES: COC(=O)c1cc(C2CC2)c2cc(Br)n(C(=O)OC(C)(C)C)c2c1]

DMAP (28 mg, 0.23 mmol) and Boc₂O (50 mg, 0.23 mmol) were added to a solution of A (64 mg, 0.22 mmol) in DCM (2 mL). The reaction was stirred at rt for 4 h. DCM and water were added to the mixture. An aqueous solution of HCl 1N was then added and the layers were separated. The organic layer was washed with brine, dried over MgSO₄ and evaporated. The residue was purified by preparative LC (irregular SiOH, 15-40 μm, 12 g GraceResolv®, mobile phase gradient: from heptane/EtOAc 100:0 to 90:10). The pure fractions were combined and evaporated under vacuum to give intermediate 115 as a white solid (68 mg, 79%).

### Intermediate H6

### 2-bromo-4-cyclopropyl-1H-indole-6-carboxylic acid

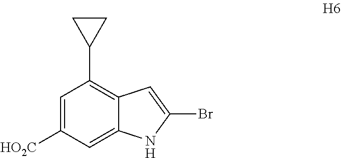 [SMILES: O=C(O)c1cc(C2CC2)c2cc(Br)[nH]c2c1]

Potassium hydroxide (34 mg, 0.51 mmol) was added to a solution of intermediate 115 (68 mg, 0.17 mmol) in EtOH (1.5 mL) and the reaction mixture was heated at 80° C. for 18 h. EtOAc and an aqueous solution of HCl 1N were added. The layers were separated and the aqueous layers were extracted with EtOAc. The organic layer was washed with brine, dried over MgSO₄, evaporated to give intermediate H6 as a yellow oil (40 mg, 83%).

### Intermediate H7

### (R)-(2-bromo-4-cyclopropyl-1H-indol-6-yl)(1-methyl-3,4-dihydroisoquinolin-2(1H)-yl)methanone

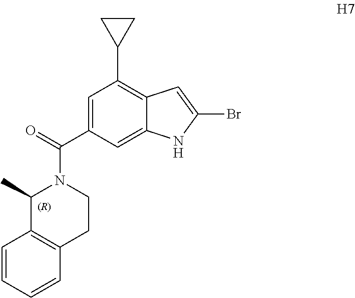

A mixture of intermediate H6 (40 mg, 0.14 mmol), (1R)-methyl-(1,2,3,4)-tetrahydroisoquinoline (25 mg, 0.17 mmol), HATU (72 mg, 0.19 mmol) and DIPEA (72 μL, 0.42 mmol) in DMF (2 mL) was stirred at rt for 20 h. The mixture was diluted in ethyl acetate, washed with an aqueous saturated solution of NaHCO₃, brine, dried over MgSO₄, filtered and evaporated. The residue was purified by preparative LC (irregular SiOH 15-40 μm, 12 g GraceResolv®, mobile phase gradient: from heptane/EtOAc from 100:0 to 50:50) to give intermediate H7 as a white solid (39 mg, 67%).

### Intermediate H8

### Ethyl (1 S,2S)-2-(4-(4-cyclopropyl-6-((R)-1-methyl-1,2,3,4-tetrahydroisoquinoline-2-carbonyl)-1H-indol-2-yl)-3-fluorophenyl)cyclopropane-1-carboxylate

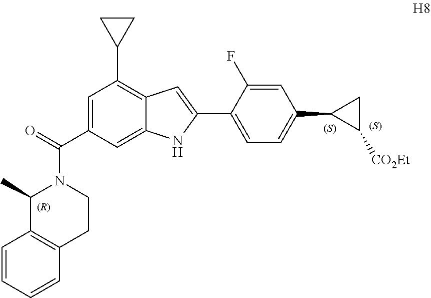

In a solution of intermediate H7 (39 mg, 95 μmol) in dioxane, were added intermediate H0 (32 mg, 95 μmol), H₂O (0.43 mL) and K₃PO₄ (69 mg, 0.32 mmol). The reaction mixture was purged with N₂ and Pd₁₁₈ (7.1 mg, 11 μmol) was added followed by a purge with N₂. The sealed tube was heated at 80° C. using one single mode microwave (Biotage Initiator EXP 60) with a power output ranging from 0 to 400 W for 30 min [fixed hold time]. EtOAc was added to the solution and the layers were separated. The organic layer was washed with brine, dried over MgSO₄, filtered, concentrated and purified by preparative LC (Irregular SiOH 15-40 μm, 12 g GraceResolv®, mobile phase: heptane/EtOAc 100:0 to 50:50). The pure fractions were collected and the solvent removed in vacuo to give intermediate H8 as a yellow solid (27 mg, 53%).

### Compound 8

### (1 S,2S)-2-(4-(4-cyclopropyl-6-((R)-1-methyl-1,2,3,4-tetrahydroisoquinoline-2-carbonyl)-1H-indol-2-yl)-3-fluorophenyl)cyclopropane-1-carboxylic acid

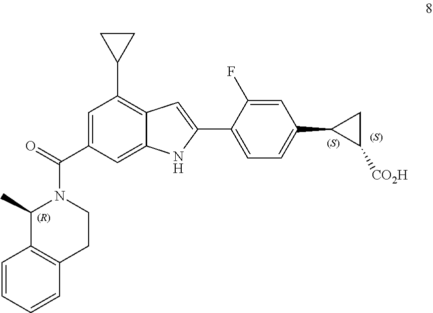

Lithium hydroxide monohydrate (12 mg, 277 μmol) was added to a solution of intermediate H8 (27 mg, 50.3 μmol) in THE (1.5 mL) and H₂O (0.5 mL) and the reaction mixture was stirred at rt for 18 h. An aqueous solution of KHSO₄ 10% was added until pH=6 and the aqueous layer was extracted with EtOAc. The organic layer was washed with water, dried over MgSO₄, filtered, evaporated and purified by preparative LC (spherical C18 25 μm, 40 g YMC-ODS-25, mobile phase gradient 0.2% aq. NH₄⁺HCO₃^{-/}MeCN from 85:15 to 45:55). The fractions containing product were combined and freeze-dried to give compound 8 as a white solid (19 mg, 74%).

6.2 Synthesis of Compound 9

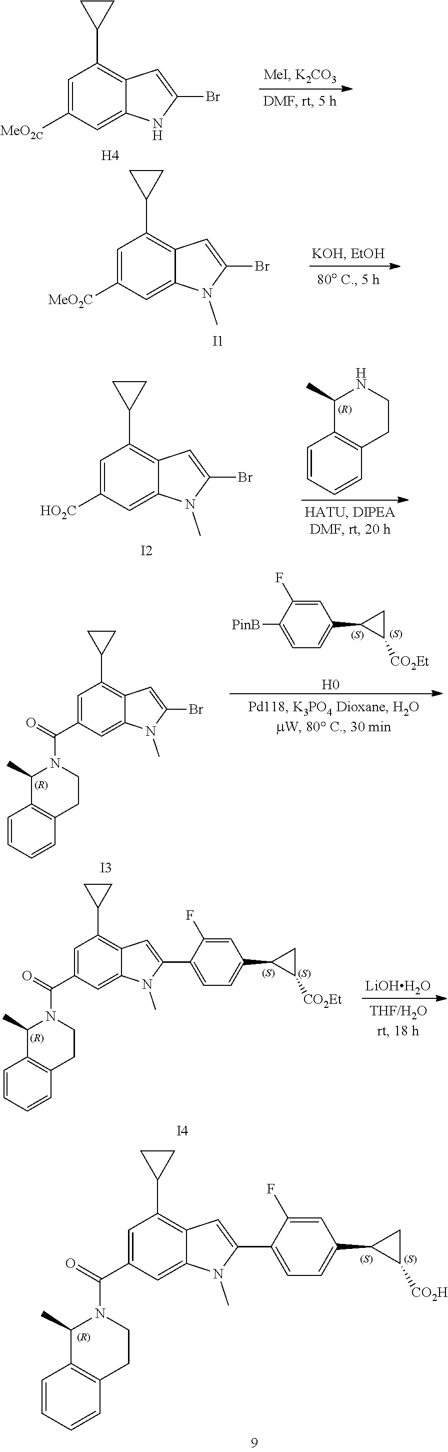

### Intermediate I1

### Methyl 2-bromo-4-cyclopropyl-1-methyl-1H-indole-6-carboxylate

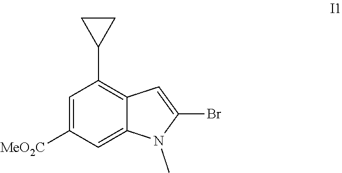 [SMILES: COC(=O)c1cc(C2CC2)c2cc(Br)n(C)c2c1]

Methyl iodine (165 μL, 2.66 mmol) was added to a solution of intermediate H4 (521 mg, 1.77 mmol) and K₂CO₃ (367 mg, 2.66 mmol) in DMF (12 mL) and the reaction mixture was stirred at rt for 3 h. Methyl iodine (28 μL, 0.44 mmol) was added to the mixture and the reaction was stirred for 2 h. EtOAc and water were added and the layers were separated. The organic layer was washed with brine, dried over MgSO₄, evaporated and purified by preparative LC (irregular SiOH 15-40 μm, 80 g GraceResolv®, mobile phase gradient: from heptane/EtOAc 100/0 to 90/10). The fractions containing product were combined and freeze-dried to give intermediate I1 as a colorless oil (170 mg, 86%).

### Intermediate I2

### 2-bromo-4-cyclopropyl-1-methyl-1H-indole-6-carboxylic acid

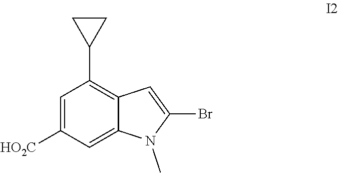 [SMILES: Cn1c(Br)cc2c(C3CC3)cc(C(=O)O)cc21]

Potassium hydroxide (300 mg, 4.55 mmol) was added to a solution of intermediate I1 (470 mg, 1.53 mmol) in EtOH (13 mL) and the reaction mixture was heated at 80° C. for 5 h. EtOAc and an aqueous solution of HCl 1N were added. The layers were separated and the organic layer was washed with brine, dried over MgSO₄, filtered and evaporated to give intermediate I2 as a white solid (432 mg, 96%).

### Intermediate I3

### (R)-(2-bromo-4-cyclopropyl-1-methyl-1H-indol-6-yl)(1-methyl-3,4-dihydroisoquinolin-2(1H)-yl)methanone

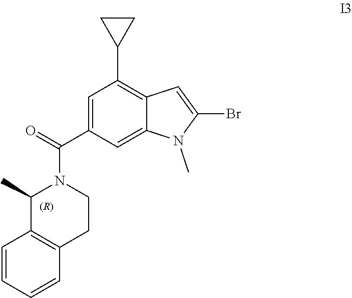

A mixture of intermediate I3 (432 mg, 1.47 mmol), (1R)-methyl-(1,2,3,4)-tetrahydroisoquinoline (259 mg, 1.76 mmol), HATU (737 mg, 1.94 mmol) and DIPEA (0.74 mL, 4.27 mmol) in DMF (21 mL) was stirred at rt for 5 h. The mixture was diluted in EtOAc, washed with an aqueous saturated solution of NaHCO₃ (twice), brine, dried over MgSO₄, evaporated and purified by preparative LC (irregular SiOH 15-40 μm, 50 g Merck, mobile phase gradient: from heptane/EtOAc from 100:0 to 50:50). The fractions containing product were combined and freeze-dried to give intermediate I3 as a white foam (612 mg, 98%).

### Intermediate I4

### Ethyl (1S,2 S)-2-(4-(4-cyclopropyl-1-methyl-6-((R)-1-methy 1-1,2,3,4-tetrahydroisoquinoline-2-carbonyl)-1H-indol-2-yl)-3-fluorophenyl)cyclopropane-1-carboxylate

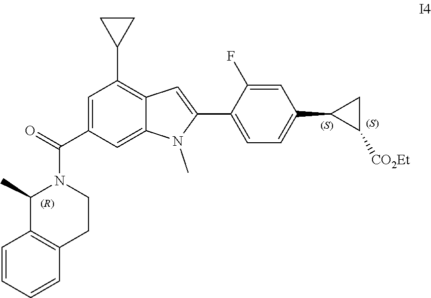

In a solution of intermediate I3 (300 mg, 0.71 mmol) in dioxane (13 mL), were added intermediate H0 (237 mg, 0.71 mmol), H₂O (3 mL) and K₃PO₄ (511 mg, 2.41 mmol). The reaction mixture was purged with N₂ and Pd₁₁₈ (53 mg, 81 μmol) was added followed by a purge with N₂. The sealed tube was heated at 80° C. using one single mode microwave (Biotage Initiator EXP 60) with a power output ranging from 0 to 400 W for 30 min [fixed hold time]. EtOAc was added to the solution and the organic layer was washed with brine, dried over MgSO₄, filtered, concentrated and purified by preparative LC (Irregular SiOH 15-40 μm, 50 g Merck, mobile phase: heptane/EtOAc 100:0 to 50:50). The pure fractions were collected and the solvent removed in vacuo to give intermediate I4 as a yellow solid (303 mg, 78%).

### Compound 9

### (1 S,2S)-2-(4-(4-cyclopropyl-1-methyl-6-((R)-1-methyl-1,2,3,4-tetrahydroisoquinoline-2-carbonyl)-1H-indol-2-yl)-3-fluorophenyl)cyclopropane-1-carboxylic acid

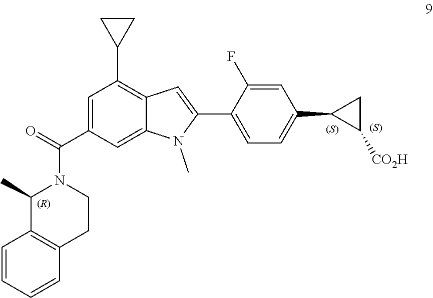

Lithium hydroxide monohydrate (127 mg, 3.03 mmol) was added to a solution of intermediate I4 (303 mg, 0.55 mmol) in THF (16 mL) and H₂O (4 mL) and the reaction mixture was stirred at rt for 18 h. An aqueous solution of KHSO4 10% was added until pH=6 and the aqueous layer was extracted with EtOAc. The organic layer was washed with water, dried over MgSO₄, filtered, evaporated and purified by preparative LC (spherical C18 25 μm, 40 g YMC-ODS-25, solid loading (Celite), mobile phase gradient 0.2% aq. NH₄⁺HCO₃⁻/MeCN from 85:15 to 45:55). The fractions containing the product were acidified with KHSO₄ 10% until pH=6 and the aqueous layer was extracted with EtOAc. The organic layer was washed with water, dried over MgSO₄, filtered and evaporated to give compound 9 as a yellow solid (212 mg, 74%).

6.3 Synthesis of Compound 10

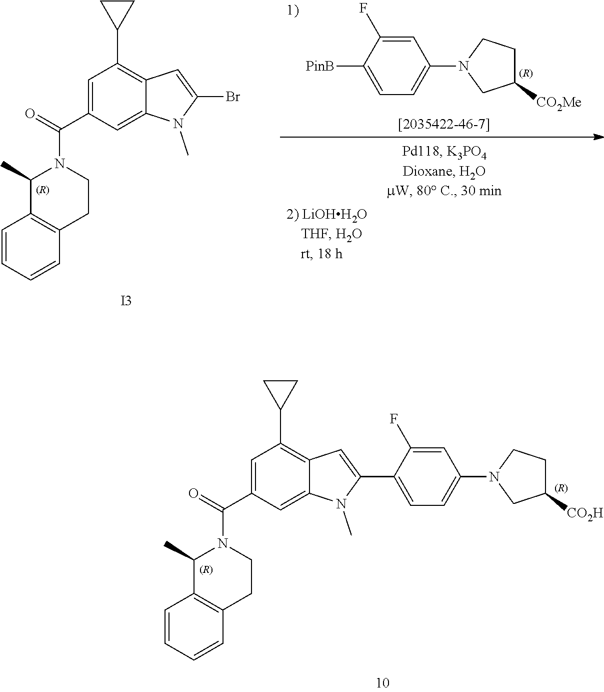

### Compound 10

### (R)-1-(4-(4-cyclopropyl-1-methyl-6-((R)-1-methyl-1,2,3,4-tetrahydroisoquinoline-2-carbonyl)-1H-indol-2-yl)-3-fluorophenyl)pyrrolidine-3-carboxylic acid

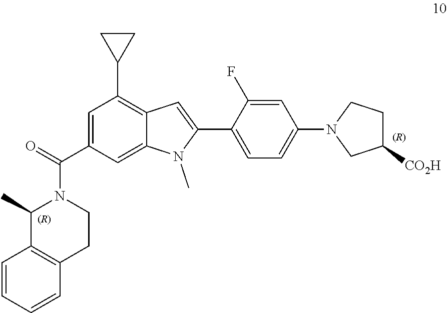

In a sealed tube, a solution of intermediate I3 (230 mg, 0.543 mmol), (3S)-1-[3-fluoro-4-(4,4,5,5-tetramethyl-1,3,2-dioxaborolan-2-yl)phenyl]-3-pyrrolidinecarboxylic acid methyl ester (228 mg, 0.652 mmol) and K₃PO₄ (346 mg, 1.63 mmol) in dioxane (8 mL) and H₂O (1.5 mL) was purged with N₂. Pd₁₁₈ (36 mg, 55 μmol) was added, the mixture was purged again with N₂ and heated at 80° C. using one single mode microwave (Biotage Initiator EXP 60) with a power output ranging from 0 to 400 W for 30 min [fixed hold time]. Lithium hydroxide monohydrate (228 mg, 5.43 mmol) was added and the reaction was stirred at 50° C. for 18 h. The solution was cooled down to rt then EtOAc and an aqueous solution of HCl (1N) were added and the aqueous layer was extracted with EtOAc. The organic layer was washed with water, dried over MgSO₄, filtered, evaporated and the residue was purified by preparative LC (Irregular SiOH 15-40 μm, 50 g Merck, mobile phase: DCM/(DCM/MeOH/AcOH 80:18:2) from 100:0 to 90:10). The fraction containing product was combined and evaporated under vacuum. The residue was purified by preparative LC (spherical C18 25 μm, 40 g YMC-ODS-25, dry loading (Celite®), mobile phase gradient 0.2% aq. NH₄⁺HCO₃⁻/MeCN from 75:25 to 35:65). The fractions containing product were freeze-dried to give compound 10 as a white solid (32 mg, 11%).

### 7. Synthesis of Azabenzimidazole

### 7.1 Synthesis of Compound 11

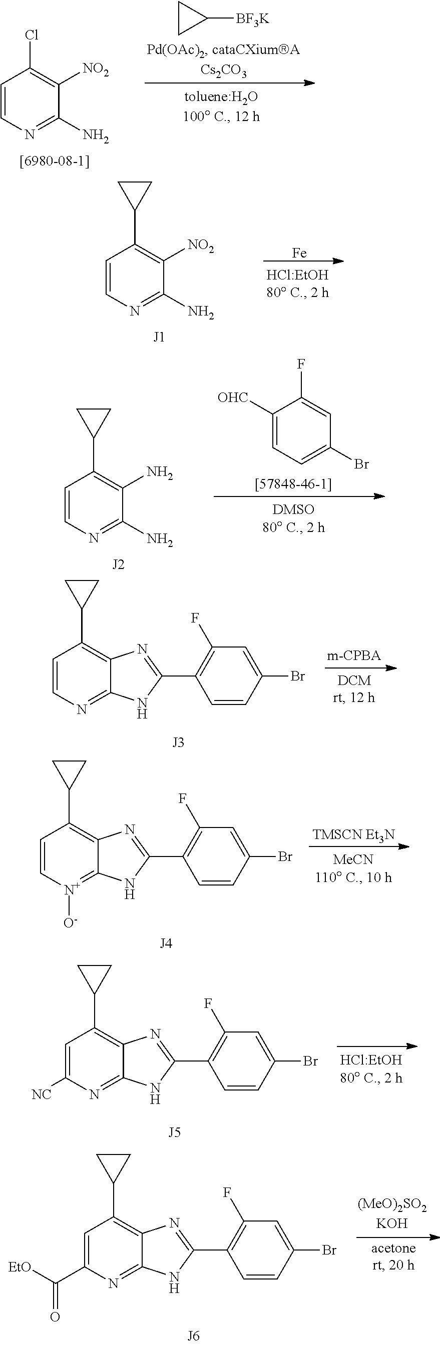

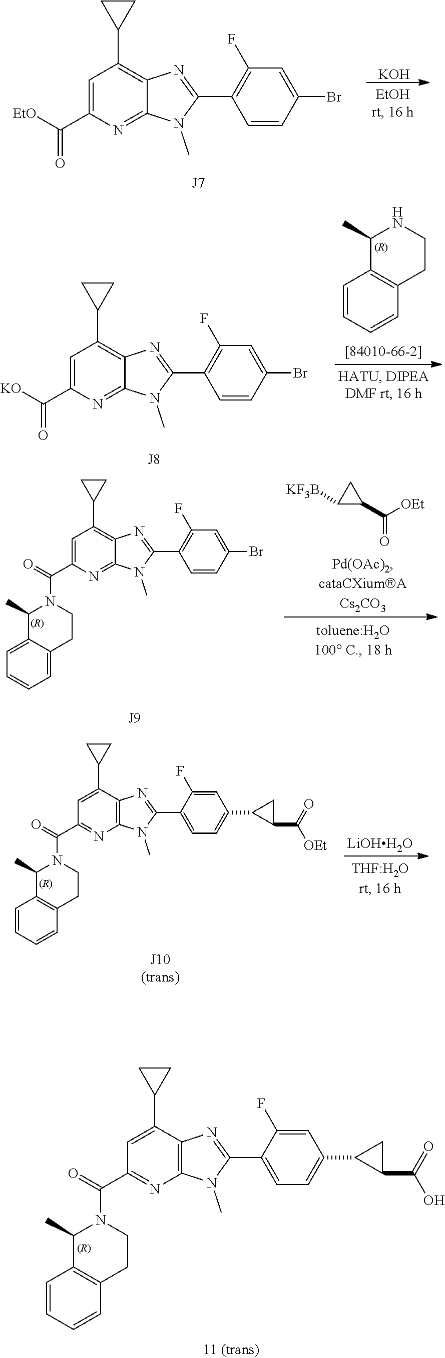

### Intermediate J1

### 4-Cyclopropyl-3-nitropyridin-2-amine

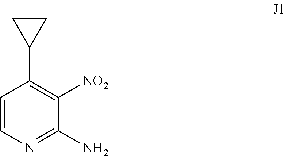 [SMILES: Nc1nccc(C2CC2)c1[N+](=O)[O-]]

A mixture of 4-chloro-3-nitro-2-pyridinamine [6980-08-1](95.0 g, 547 mmol), potassium cyclopropyltrifluoroborate [1065010-87-8](162 g, 1.09 mol), palladium acetate (2.46 g, 11.0 mmol), cesium carbonate (535 g, 1.64 mol) and cataCXium® A (5.89 g, 16.4 mmol) in H₂O (250 mL) and toluene (2.5 L) was stirred at 100° C. for 12 h. The reaction mixture was filtered through a pad of Celite® and the filter-cake was washed with EtOAc. The layers were separated and the aqueous phase was extracted with EtOAc (3×500 mL). The combined organic extracts were concentrated in vacuo. The crude mixture was purified by column chromatography (SiO₂, mobile phase gradient: petroleum ether/EtOAc from 20:1 to 3:1) to afford intermediate J1 (70 g, 71%) as a yellow solid.

### Intermediate J2

### 4-Cyclopropylpyridine-2,3-diamine

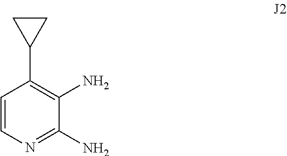 [SMILES: Nc1nccc(C2CC2)c1N]

To a solution of intermediate J1 (77.0 g, 429.7 mmol) in HCl (80 mL) and EtOH (1 L) was added iron powder (132 g, 2.36 mol) portionwise. The reaction mixture was stirred at 80° C. for 2 h. The reaction mixture was filtered through a pad of Celite® and the filter-cake was washed with DCM. The filtrate was concentrated in vacuo to afford intermediate J2 (60 g, 94%) as a light yellow solid which was used into the next step without further purification.

### Intermediate J3

### 2-(4-Bromo-2-fluorophenyl)-7-cyclopropyl-3H-imidazo[4,5-b]pyridine

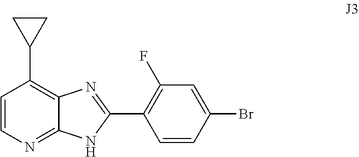 [SMILES: Fc1cc(Br)ccc1-c1nc2c(C3CC3)ccnc2[nH]1]

A mixture of intermediate J2 (53.0 g, 355 mmol) and 4-bromo-2-fluoro-5-methylbenzaldehyde [57848-46-1] (86.5 g, 426 mmol) in DMSO (530 mL) was stirred at 80° C. for 2 h. The reaction mixture was poured out into water (6 L). The precipitate was filtered off and the solid was washed with H₂O (3×200 mL). The crude product was triturated in DCM (2×100 mL) to afford intermediate J3 (85 g, 72%) as a pale yellow solid.

### Intermediate J4

### 2-(4-Bromo-2-fluorophenyl)-7-cyclopropyl-3H-imidazo[4,5-b]pyridin-4-ium-4-olate

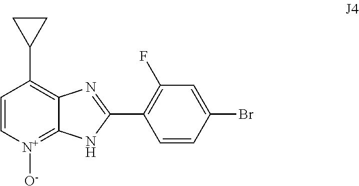 [SMILES: [O-][n+]1ccc(C2CC2)c2nc(-c3ccc(Br)cc3F)[nH]c21]

To a solution of intermediate J3 (20.0 g, 60.2 mmol) in DCM (100 mL) was added m-CPBA (12.2 g, 60.2 mmol). The reaction mixture was stirred at rt for 12 h. The reaction mixture was washed with an aqueous solution of Na₂S₂O³ (2×100 mL) and an aqueous solution of NaHCO₃ (3×100 mL), dried over Na₂SO₄, filtered and concentrated in vacuo to afford intermediate J4 (15 g, 70%) as a light yellow solid.

### Intermediate J5

### 2-(4-Bromo-2-fluorophenyl)-7-cyclopropyl-3H-imidazo[4,5-b]pyridine-5-carbonitrile

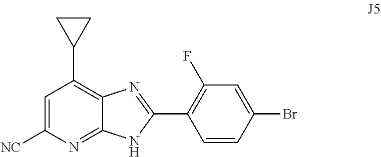 [SMILES: N#Cc1cc(C2CC2)c2nc(-c3ccc(Br)cc3F)[nH]c2n1]

A mixture of intermediate J4 (14.0 g, 40.2 mmol), TMSCN (23.9 g, 241.3 mmol) and Et₃N (16.3 g, 160.8 mmol) in MeCN (75 mL) was stirred at 110° C. for 10 h. The solvent was evaporated in vacuo. The crude mixture was purified by column chromatography (SiO₂, mobile phase gradient: DCM/MeOH from 1:0 to 99.5:0.5) to afford intermediate J5 (11 g, 73%, 95% purity) as a light yellow solid.

### Intermediate J6

### Ethyl 2-(4-bromo-2-fluorophenyl)-7-cyclopropyl-3H-imidazo[4,5-b]pyridine-5-carboxylate

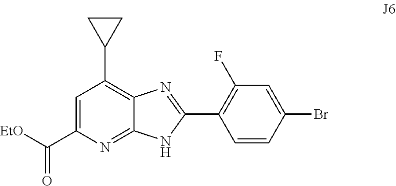 [SMILES: CCOC(=O)c1cc(C2CC2)c2nc(-c3ccc(Br)cc3F)[nH]c2n1]

A mixture of intermediate J5 (15.0 g, 42.0 mmol) in HCl in EtOH (4.0 M, 100 mL) was stirred at 80° C. for 2 h. The solvent was evaporated in vacuo. The crude mixture was purified by preparative HPLC (column: Phenomenex luna C18 250*50 mm*10 um, mobile phase gradient: H₂O (+0.1% TFA)/MeCN from 70:30 to 35:65). The residue was basified with an aqueous solution of NaHCO₃ until pH 7-8. The layers were separated and the organic phase was dried over Na₂SO₄, filtered and concentrated in vacuo to afford intermediate J6 (8 g, 45%, 95% purity) as a light yellow solid.

### Synthesis of Intermediate J7 and J7′

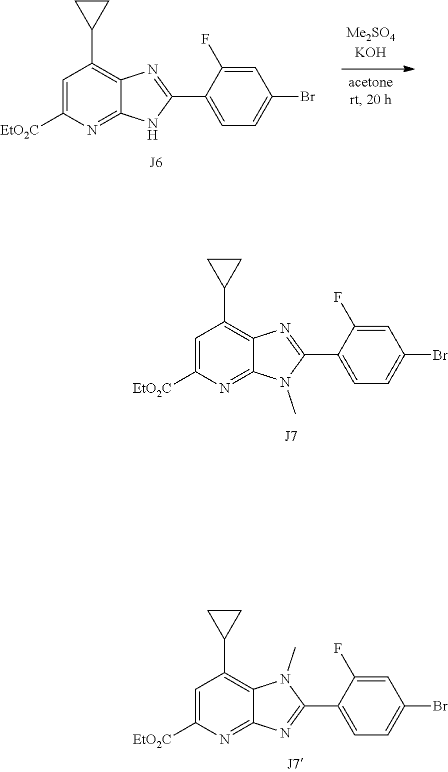 [SMILES: CCOC(=O)c1cc(C2CC2)c2nc(-c3ccc(Br)cc3F)[nH]c2n1 ; CCOC(=O)c1cc(C2CC2)c2nc(-c3ccc(Br)cc3F)n(C)c2n1 ; CCOC(=O)c1cc(C2CC2)c2c(n1)nc(-c1ccc(Br)cc1F)n2C]

Dimethylsulfate (737 μL, 7.79 mmol) was added to a mixture of intermediate J6 (3.0 g, 7.4 mmol) and potassium hydroxide (437 mg, 7.79 mmol) in acetone (42 mL). The reaction mixture was stirred at rt for 20 h. The reaction mixture was evaporated. Water and DCM were added to the residue. The aqueous layer was extracted with DCM. The combined organic layers were dried over MgSO₄, filtered and evaporated in vacuo. The residue was purified by preparative LC (irregular SiOH, 15-40 μm, 330 g GraceResolv®, mobile phase gradient: from Heptane/EtOAc 90/10 to 40/60). The fraction containing intermediate J7 & intermediate J7′ were evaporated separately to give 2 fractions. First fraction containing intermediate J7′ as a yellow solid (477 mg, 15%) and a second fraction which was taken up in MeCN and evaporated to give intermediate J7 as a colorless gum which crystallized upon standing (1.74 g, 56%).

### Intermediate J8

### Potassium 2-(4-bromo-2-fluorophenyl)-7-cyclopropyl-3-methyl-3H-imidazo[4,5-b]pyridine-5-carboxylate

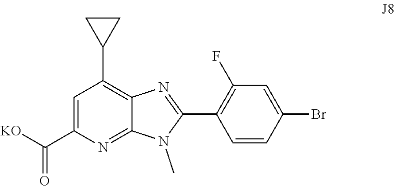

A mixture of intermediate J7 (1.74 g, 4.16 mmol) and potassium hydroxide (467 mg, 8.32 mmol) in EtOH (55 mL) was stirred at rt for 16 h. The reaction mixture was filtered over frit. The solid was washed with Et₂O and dried under high vacuum at 50° C. for 2 h to afford intermediate J8 (1.48 g, 91%) as a white solid.

### Intermediate J9

### (1R)-2-[2-(4-Bromo-2-fluorophenyl)-7-cyclopropyl-3-methyl-3H-imidazo[4,5-b]pyridine-5-carbonyl]-1-methyl-1,2,3,4-tetrahydroisoquinoline

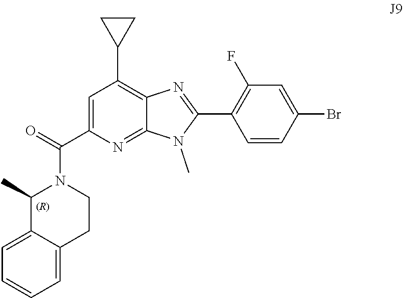

To a mixture of intermediate J8 (1.48 g, 3.79 mmol), (R)-1-methyl-1,2,3,4-tetrahydroisoquinoline [84010-66-2] (838 mg, 5.69 mmol) and DIPEA (1.67 mL, 9.70 mmol) in DMF (20 mL) was added HATU (2.60 g, 6.83 mmol). The reaction mixture was stirred at rt for 16 h. The reaction mixture was diluted with H₂O and extracted with EtOAc. The combined organic extracts were washed with brine, dried over MgSO₄, filtered and the solvent was evaporated in vacuo. The crude mixture was purified by preparative LC (regular SiOH, 15-40 μm, GraceResolv® 80 g, mobile phase gradient: heptane/EtOAc from 90:10 to 50:50) to afford intermediate J9 (1.84 g, 88%) as a white solid.

### Intermediate J10

### Ethyl trans 2-(4-{7-cyclopropyl-3-methyl-5-[(1R)-1-methyl-1,2,3,4-tetrahydroisoquinoline-2-carbonyl]-3H-imidazo[4,5-b]pyridin-2-yl}-3-fluorophenyl)cyclopropane-1-carboxylate

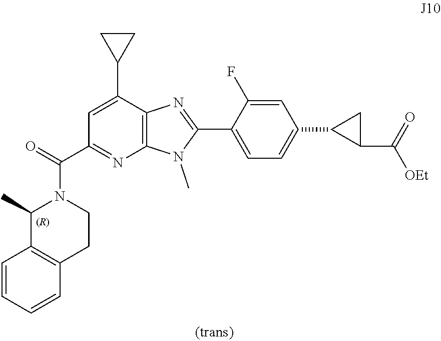

To a mixture of intermediate J9 (200 mg, 362 μmol, 94% purity), [1612792-88-7] (cis:trans 14:86) (159 mg, 724 μmol) and cesium carbonate (354 mg, 1.09 mmol) in toluene (4 mL) and H₂O (0.4 mL) under a nitrogen atmosphere were added Catacxium®A (31.1 mg, 86.9 μmol) and palladium acetate (13.0 mg, 57.9 μmol). The reaction mixture was purged with nitrogen and stirred at 100° C. for 18 h. The reaction mixture was diluted with water and EtOAc. The layers were separated and the aqueous phase was extracted with EtOAc (twice). The combined organic extracts were dried over MgSO₄, filtered and concentrated in vacuo. The crude mixture was purified by preparative LC (irregular SiOH, 15-40 μm, 40 g GraceResolv®, mobile phase gradient: heptane/EtOAc from 90:10 to 40:60) to afford intermediate J10 (154 mg, 77%) as an off-white solid.

### Compound 11 (trans)

### Trans 2-(4-{7-Cyclopropyl-3-methyl-5-[(1R)-1-methyl-1,2,3,4-tetrahydroisoquinoline-2-carbonyl]-3H-imidazo[4,5-b]pyridin-2-yl}-3-fluorophenyl)cyclopropane-1-carboxylic acid

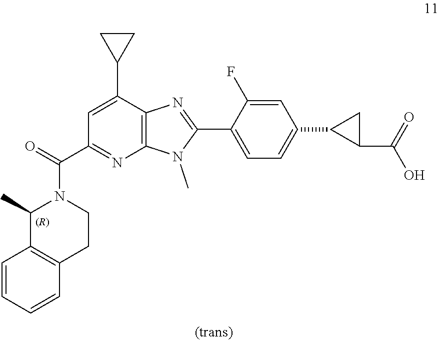

Lithium hydroxide monohydrate (35.1 mg, 0.84 mmol) was added to a solution of intermediate J10 (154 mg, 279 μmol) in THE (2.3 mL) and H₂O (0.9 mL). The reaction mixture was stirred at rt for 16 h. The reaction mixture was diluted with brine and a 10% aqueous solution of KHSO₄ was added. The aqueous phase was extracted with EtOAc. The combined organic extracts were washed with H₂O, dried over MgSO₄, filtered and concentrated in vacuo. The crude mixture was taken-up in MeOH and concentrated in vacuo. The residue was triturated in Et₂O. The solid was filtered off and dried under high vacuum at 50° C. for 20 h to give compound 11 (92 mg, 63%) as a white solid.

7.2 Synthesis of Compound 12

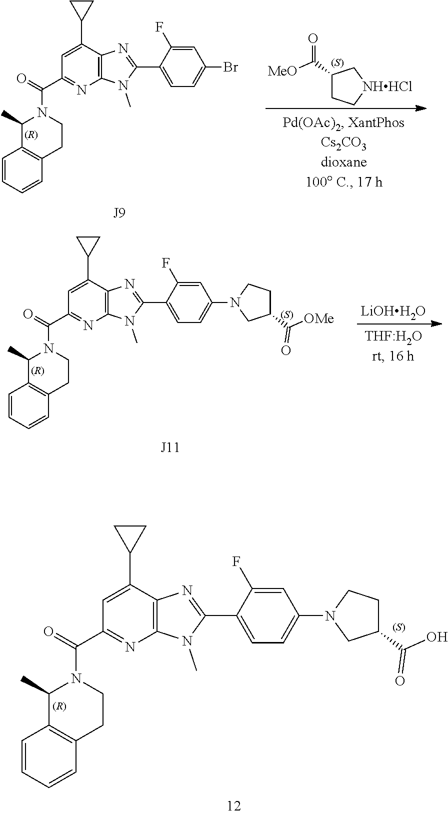

### Intermediate J11

### Methyl (3 S)-1-(4-{7-cyclopropyl-3-methyl-5-[(1R)-1-methyl-1,2,3,4-tetrahydroisoquinoline-2-carbonyl]-3H-imidazo[4,5-b]pyridin-2-yl}-3-fluorophenyl)pyrrolidine-3-carboxylate

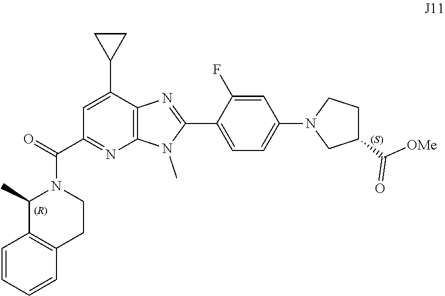

A sealed tube was charged with intermediate J9 (165 mg, 318 μmol), (S)-methyl pyrrolidine-3-carboxylate hydrochloride [1099646-61-3] (63.1 mg, 381 μmol), cesium carbonate (311 mg, 0.95 mmol) and XantPhos (18.4 mg, 31.8 μmol) and purged with nitrogen. 1,4-Dioxane (5 mL) was added and the mixture was purged again with nitrogen. Palladium acetate (7.13 mg, 31.8 μmol) was added. The reaction mixture was purged with nitrogen and stirred at 100° C. for 17 h. The reaction mixture was diluted with EtOAc and H₂O. The layers were separated and the aqueous phase was extracted with EtOAc (twice). The combined organic extracts were washed with brine, dried over MgSO₄, filtered and the solvent was evaporated in vacuo. The crude mixture was purified by preparative LC (irregular SiOH, 15-40 μm, 40 g GraceResolv®, mobile phase gradient: heptane/EtOAc from 90:10 to 40:60) to afford intermediate J11 (131 mg, 70%, 96% purity) as a yellow foam.

### Compound 12

### Methyl (3S)-1-(4-{7-cyclopropyl-3-methyl-5-[(1R)-1-methyl-1,2,3,4-tetrahydroisoquinoline-2-carbonyl]-3H-imidazo[4,5-b]pyridin-2-yl}-3-fluorophenyl)pyrrolidine-3-carboxylic acid

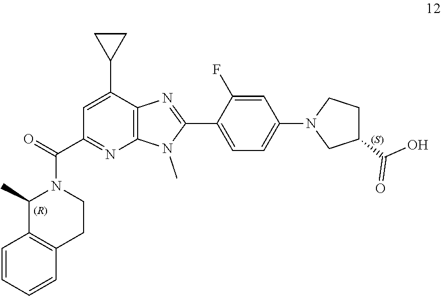

Lithium hydroxide monohydrate (29.1 mg, 0.69 mmol) was added to a solution of intermediate J11 (131 mg, 231 μmol) in THF (1.9 mL) and H₂O (0.7 mL). The reaction mixture was stirred at rt for 16 h. The reaction mixture was diluted with brine and a 10% aqueous solution of KHSO₄ was added. The aqueous phase was extracted with EtOAc. The combined organic extracts were washed with H₂O, dried over MgSO₄, filtered and concentrated in vacuo. The crude mixture was taken-up in MeCN and concentrated in vacuo. The residue was triturated in Et₂O. The solid was filtered off and dried under high vacuum at 50° C. for 20 h to give compound 12 (89 mg, 70%) as a pale yellow solid.

7.3 Synthesis of Compound 13

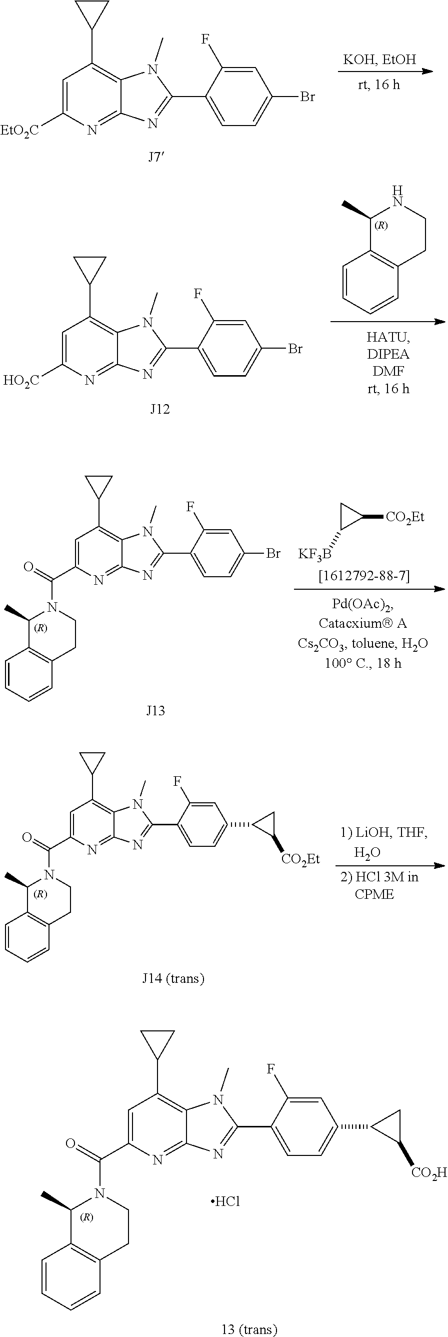

### Intermediate J12

### 2-(4-bromo-2-fluorophenyl)-7-cyclopropyl-1-methyl-1H-imidazo[4,5-b]pyridine-5-carboxylic acid

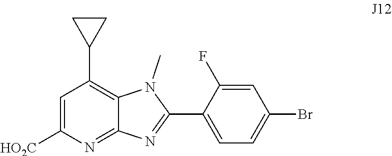 [SMILES: Cn1c(-c2ccc(Br)cc2F)nc2nc(C(=O)O)cc(C3CC3)c21]

A mixture of intermediate J7′ (477 mg, 1.14 mmol) and potassium hydroxide (128 mg, 2.28 mmol) in EtOH (15 mL) was stirred at rt for 16 h. The mixture was evaporated in vacuo, the residue was taken up in water and the mixture was acidified with an aq. solution of HCl (1N). The aqueous layer was extracted with DCM to give intermediate J12 as a yellow gum (200 mg, 45%).

### Intermediate J13

### (R)-(2-(4-bromo-2-fluorophenyl)-7-cyclopropyl-1-methyl-1H-imidazo[4,5-b]pyridin-5-yl)(1-methyl-3,4-dihydroisoquinolin-2(1H)-yl)methanone

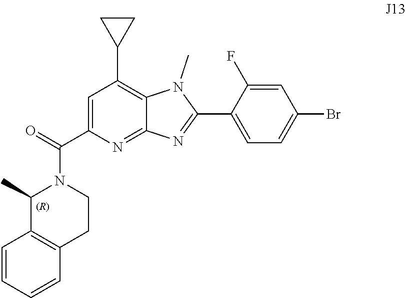

To a mixture of intermediate J12 (200 mg, 0.513 mmol) and (JR)-methyl-(1,2,3,4)-tetrahydroisoquinoline (113 mg, 0.769 mmol) and DIPEA (226 μL, 1.31 mmol) in DMF (3 mL) was added HATU (351 mg, 0.923 mmol) at rt. The resulting mixture was stirred at rt for 16 h. Water was added to the mixture and the product was extracted with EtOAc. The combined organic layers were washed with brine, dried over MgSO₄, filtered and the solvent was removed in vacuo. The residue was purified by preparative LC (regular SiOH, 15-40 μm, GraceResolv®12 g, mobile phase gradient: from heptane/EtOAc 90/10 to 70/30). The fractions containing product were combined and evaporated in vacuo to give intermediate J13 as a white foam (281 mg, impure, used like this in the next step).

### Intermediate J14 (trans)

### Ethyl (trans)-2-(4-(7-cyclopropyl-1-methyl-5-((R)-1-methyl-1,2,3,4-tetrahydroisoquinoline-2-carbonyl)-1H-imidazo[4,5-b]pyridin-2-yl)-3-fluorophenyl)cyclopropane-1-carboxylate

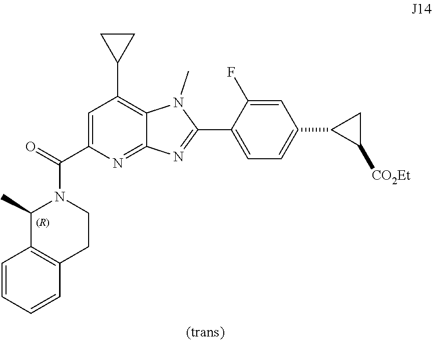

To a mixture of intermediate J13 (139 mg, 0.268 mmol), (T-4)-borate (1-) [rel-(1R,2R)-2-(ethoxycarbonyl)cyclopropyl]trifluoro-, potassium (1:1) (1612792-88-7) (118 mg, 0.535 mmol) and Cs₂CO₃ (262 mg, 0.803 mmol) in toulene (3 mL) and H₂O (0.3 mL) under a nitrogen atmosphere was added Catacxium® A (23 mg, 0.064 mmol) and Pd(OAc)₂ (10 mg, 0.043 mmol). The mixture was purged with nitrogen and stirred at 100° C. for 18 h. Water and EtOAc were added. The layers were separated and the aqueous layer was extracted with EtOAc. The combined organic layers were dried over MgSO₄, filtered, evaporated. The residue was purified by preparative LC (irregular SiOH, 15-40 μm, GraceResolv® 12 g, mobile phase gradient: from DCM/EtOAc 100/0 to 70/30). The fractions containing product were combined and evaporated in vacuo to give intermediate J14 as a colorless gum (58 mg, 39%).

### Compound 13

### (Trans)-2-(4-(7-cyclopropyl-1-methyl-5-((R)-1-methyl-1,2,3,4-tetrahydroisoquinoline-2-carbonyl)-1H-imidazo[4,5-b]pyridin-2-yl)-3-fluorophenyl)cyclopropane-1-carboxylic acid

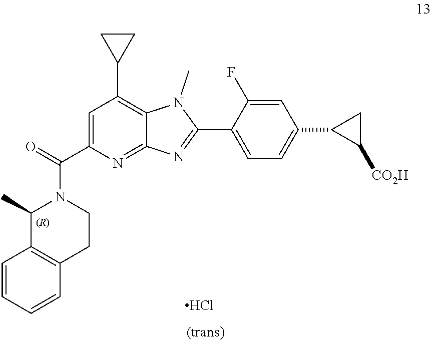

Lithium hydroxide monohydrate (13 mg, 0.32 mmol) was added to a solution of intermediate J14 (58 mg, 0.11 mmol) in THF (0.9 mL) and H₂O (0.3 mL) and the reaction mixture was stirred at rt for 18 h. HCl 3M in CPME (0.120 mL, 0.357 mmol) was added, the mixture was stirred at rt for 30 min and evaporated in vacuo. The residue was purified by Reverse phase (Stationary phase: YMC-actus Triart C18 10 μm 30*150 mm, Mobile phase: Gradient from 65% aq. TFA 0.1% pH=2.5, 35% MeCN to 25% aq. TFA pH=2.5, 75% MeCN). The fractions containing product were combined and evaporated in vacuo. The resulting colorless gum was taken up in THF (1 mL). Then HCl3M in CPME (0.5 mL) was added and the solution was stirred at rt for 1 h. The solution was evaporated in vacuo. The residue was triturated in Et₂O, filtered and dried to give compound 13 as a white solid (34 mg, 58%).

### 8. Synthesis of Benzimidazoles

### 8.1 Synthesis of Intermediates K₁

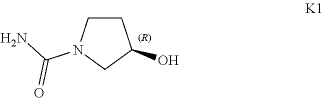

### Intermediate K1

### (3R)-3-Hydroxypyrrolidine-1-carboxamide

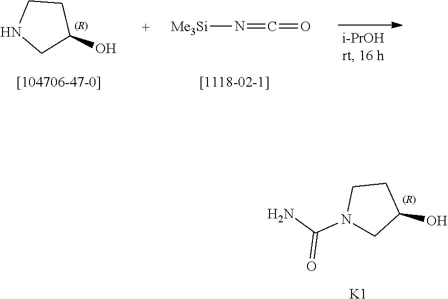

Trimethylsilyl isocyanate [1118-02-1] (8.0 mL, 64.3 mmol) was added dropwise to a solution of (R)-3-hydroxypyrrolidine [104706-47-0] (4.00 g, 45.9 mmol) in i-PrOH (110 mL). The reaction mixture was stirred at rt for 16 h. The mixture was concentrated in vacuo until precipitation was observed (˜half of the solvent). The solid was filtered off, washed with i-PrOH and dried to afford intermediate K1 (4.6 g, 77%) as a white solid.

8.2 Synthesis of Compound 14

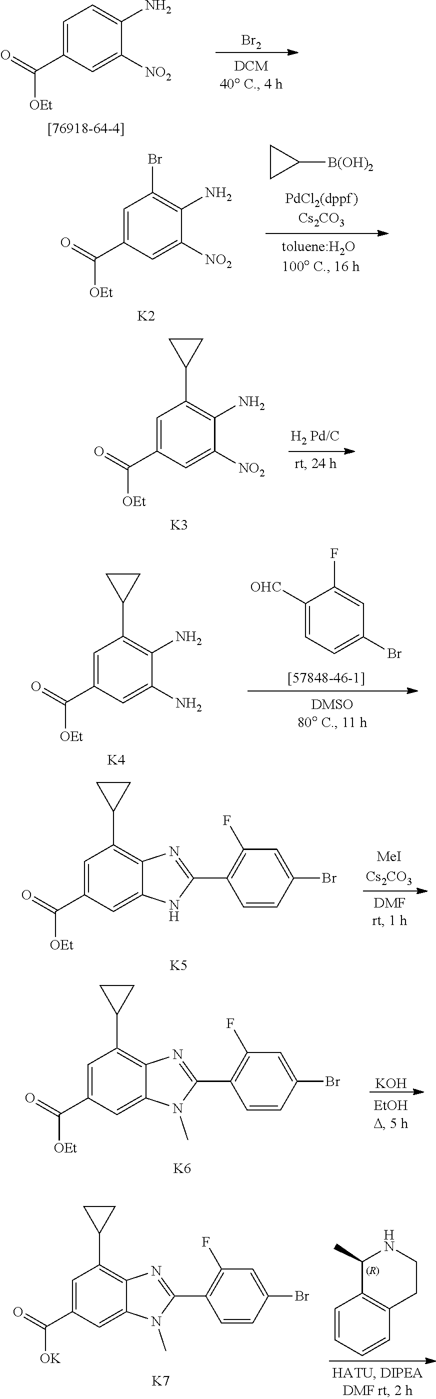

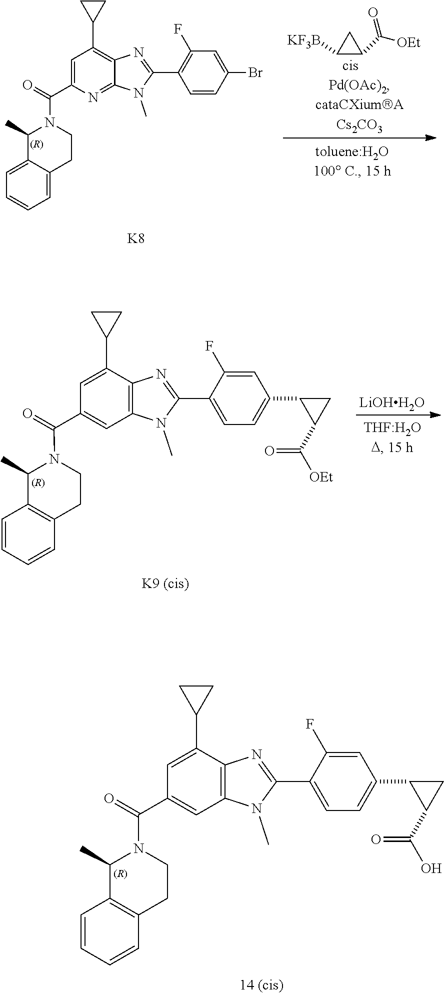

### Intermediate K2

### Ethyl 4-amino-3-bromo-5-nitrocybenzoate

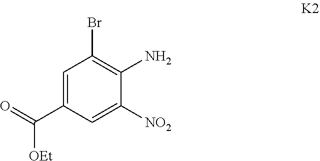 [SMILES: CCOC(=O)c1cc(Br)c(N)c([N+](=O)[O-])c1]

To a solution of ethyl 4-amino-3-nitrobenzoate [76918-64-4] (55.0 g, 261 mmol) in DCM (2 L) was added bromine (62.7 g, 392 mmol) dropwise. The reaction mixture was stirred at 40° C. for 4 h. The reaction mixture was poured out into a saturated aqueous solution of Na₂SO₃ (2 L) under stirring. The layers were separated and the aqueous phase was extracted with DCM (2×1 L). The combined organic extracts were washed with an aqueous solution of NaHCO₃ (1 L), dried over Na₂SO₄, filtered and concentrated in vacuo. The residue was dissolved in DCM (1.5 L). The suspension was filtered through a pad of Celite®. The filtrate was concentrated in vacuo to afford intermediate K2 (76 g, quant.) as a yellow solid.

### Intermediate K3

### Ethyl 4-amino-3-cyclopropyl-5-nitrobenzoate

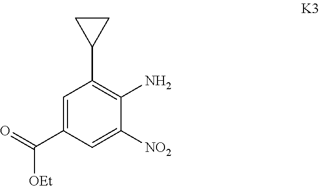 [SMILES: CCOC(=O)c1cc(C2CC2)c(N)c([N+](=O)[O-])c1]

To a solution of cesium carbonate (103 g, 316 mmol) in H₂O (760 mL) was added a solution of intermediate K2 (76 g, 263 mmol) in toluene (760 mL). The mixture was purged with nitrogen for 30 min. Cyclopropylboronic acid [411235-57-9] (45.2 g, 526 mmol) and [1,1′-bis(diphenyl-phosphino)ferrocene]dichloropalladium(II) (19.2 g, 26.3 mmol) were added. The reaction mixture was stirred at 100° C. for 16 h under nitrogen atmosphere. The reaction mixture was filtered through a pad of Celite® and the filter-cake was washed with DCM (1.5 L). The filtrate was diluted with H₂O. The layers were separated and the aqueous phase was extracted with DCM (2×1 L). The combined organic extracts were washed with H₂O (1 L), dried over Na₂SO₄, filtered and the solvent was evaporated in vacuo. The residue was dissolved in DCM (1.5 L). The suspension was filtered through a pad of Celite®. The filtrate was concentrated in vacuo to afford intermediate K3 (68 g, crude) as a yellow solid.

### Intermediate K4

### Ethyl 3,4-diamino-5-cyclopropylbenzoate

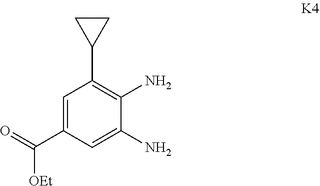 [SMILES: CCOC(=O)c1cc(N)c(N)c(C2CC2)c1]

To a solution of intermediate K3 (68.0 g, 272 mmol) in EtOH (800 mL) was added Pd/C (10 wt. % 10.0 g, 9.39 mmol). The reaction mixture was stirred at rt for 24 h under H₂ atmosphere. The reaction mixture was filtered through a pad of Celite® and the filter cake was washed with EtOH (1.5 L). The filtrate was concentrated in vacuo to afford intermediate K4 (50.0 g, 84% over 2 steps) as a black solid that was used in the next step without further purification.

### Intermediate K5

### Ethyl 2-(4-bromo-2-fluorophenyl)-4-cyclopropyl-1H-1,3-benzodiazole-6-carboxylate

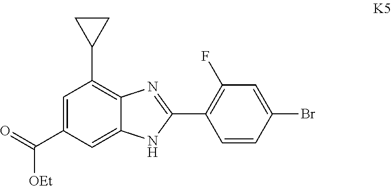 [SMILES: CCOC(=O)c1cc(C2CC2)c2nc(-c3ccc(Br)cc3F)[nH]c2c1]

A solution of intermediate K4 (45 g, 204 mmol) and 4-bromo-2-fluorobenzaldehyde [57848-46-1] (46.0 g, 227 mmol) in DMSO (450 mL) was stirred at 80° C. for 8 h. Additional amount of 4-bromo-2-fluorobenzaldehyde (5.00 g, 24.6 mmol) was added and the reaction mixture was stirred at 80° C. for another 3 h. The reaction mixture was poured out into water (3 L) under stirring. The aqueous phase was extracted with EtOAc (3×1.5 L). The combined organic extracts were washed with H₂O (2×1 L), dried over Na₂SO₄, filtered and evaporated in vacuo. The crude mixture was purified by recrystallization from EtOAc (1 L) to afford intermediate K5 (36 g, 44%) as a brown solid.

### Intermediate K6

### Ethyl 2-(4-bromo-2-fluorophenyl)-4-cyclopropyl-1-methyl-1H-1,3-benzodiazole-6-carboxylate

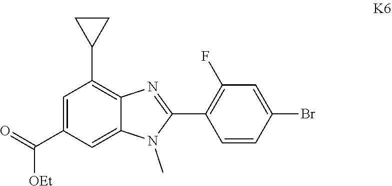 [SMILES: CCOC(=O)c1cc(C2CC2)c2nc(-c3ccc(Br)cc3F)n(C)c2c1]

To a mixture of intermediate K5 (500 mg, 1.24 mmol) and cesium carbonate (1.41 g, 4.34 mmol) in DMF (2.5 mL) was added dropwise methyl iodide (116 μL, 1.86 mmol). The reaction mixture was stirred at rt for 1 h. The reaction mixture was diluted with H₂O and EtOAc. The layers were separated and the aqueous phase was extracted with EtOAc. The combined organic extracts were washed with brine, dried over MgSO₄, filtered and evaporated to dryness. The crude mixture was purified by flash chromatography over silica gel (Puriflash Interchim® 40 g, 30 μm, mobile phase gradient: heptane/EtOAc from 100:0 to 80:20) to afford intermediate K6 (0.36 g, 70%) as a white solid.

### Intermediate K7

### Potassium 2-(4-bromo-2-fluorophenyl)-4-cyclopropyl-1-methyl-1H-1,3-benzodiazole-6-carboxylate

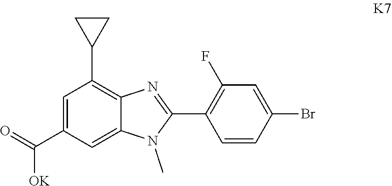

A mixture of intermediate K6 (2.26 g, 5.42 mmol) and potassium hydroxide (912 mg, 16.3 mmol) in EtOH (70 mL) was stirred under reflux for 5 h. The reaction mixture was cooled to 0° C. and acidified with a 3N aqueous solution of HCl. The precipitate was filtered off and dried under vacuum to afford intermediate K7 (1 g, 43%) as a beige solid.

### Intermediate K8

### (1R)-2-[2-(4-Bromo-2-fluorophenyl)-4-cyclopropyl-1-methyl-1H-1,3-benzodiazole-6-carbonyl]-1-methyl-1,2,3,4-tetrahydroisoquinoline

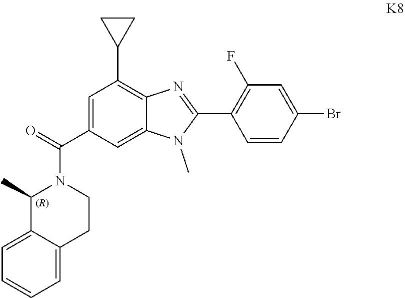

To the solution of intermediate K7 (0.61 g, 1.43 mmol) and (R)-1-methyl-1,2,3,4-tetrahydrosioquinoline [84010-66-2] (252 mg, 1.71 mmol) in DMF (18 mL) were added DIPEA (0.74 mL, 4.28 mmol) and HATU (0.71 g, 1.86 mmol). The reaction mixture was stirred at rt for 2 h. The reaction mixture was poured out slowly into water and extracted with EtOAc. The combined organic extracts were washed with H₂O and brine, dried over MgSO₄, filtered and evaporated to dryness. The crude mixture was purified by flash chromatography over silica gel (Puriflash Interchim® 40 g, 30 μm, mobile phase gradient: heptane/EtOAc from 100:0 to 60:40) to afford intermediate K8 (416 mg, 56%) as a beige solid.

### Intermediate K9

### Ethyl cis-2-(4-{4-cyclopropyl-1-methyl-6-[(1R)-1-methyl-1,2,3,4-tetrahydroisoquinoline-2-carbonyl]-1H-1,3-benzodiazol-2-yl}-3-fluorophenyl)cyclopropane-1-carboxylate

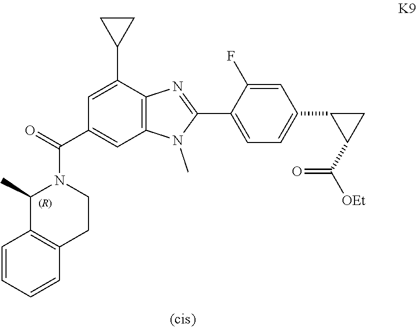

A mixture of intermediate K8 (0.25 g, 482 μmol), (T-4)-borate (1-) [rel-(1R,2R)-2-(ethoxycarbonyl)cyclopropyl]trifluoro-, potassium (1:1) (1612792-88-7) (cis:trans 86:14) (256 mg, 1.21 mmol) and cesium carbonate (0.47 g, 1.45 mmol) in toluene (5.2 mL) and water (0.53 mL) was purged with nitrogen for 10 min. Catacxium®A (41.5 mg, 116 μmol) and palladium acetate (17.3 mg, 77.2 μmol) were added. The reaction mixture was purged with nitrogen for 5 min and stirred at 100° C. for 15 h. The reaction mixture was diluted with H₂O and EtOAc. The mixture was filtered through a pad of Celite® and washed with EtOAc. The layers were separated and the organic phase was washed with H₂O and brine, dried over MgSO₄, filtered and evaporated to dryness. The crude mixture was purified by flash chromatography over silica gel (Puriflash Interchim® 80 g, 30 μm, mobile phase gradient: heptane/DCM, from 100:0 to 40:60) to afford intermediate K9 (0.2 g, 75%) as a beige solid.

### Compound 14

### Cis-2-(4-{4-cyclopropyl-1-methyl-6-[(1R)-1-methyl-1,2,3,4-tetrahydroisoquinoline-2-carbonyl]-1H-1,3-benzodiazol-2-yl}-3-fluorophenyl)cyclopropane-1-carboxylic acid

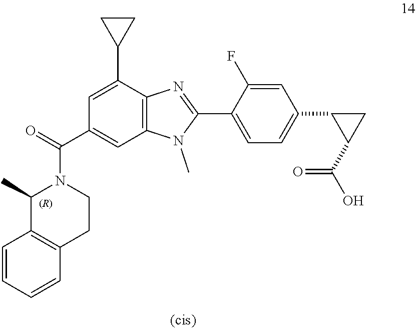

A mixture of intermediate K9 (0.19 g, 0.34 mmol) and lithium hydroxide monohydrate (0.10 g, 2.41 mmol) in THE (8.5 mL) and water (2 mL) was stirred under reflux for 15 h. An aqueous solution of citric acid (463 mg in 5 mL of H₂O) was added. The layers were separated and the aqueous phase was extracted with EtOAc. The combined organic extracts were washed with brine, dried over MgSO₄, filtered and evaporated to dryness to give compound 14 (0.18 g, quant.) as a beige solid.

8.3 Synthesis of Compound 15

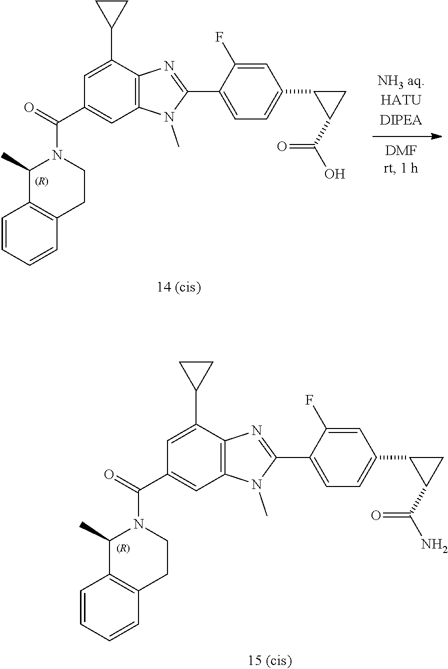

### Compound 15

### Cis-2-(4-{4-cyclopropyl-1-methyl-6-[(1R)-1-methyl-1,2,3,4-tetrahydroisoquinoline-2-carbonyl]-1H-1,3-benzodiazol-2-yl}-3-fluorophenyl)cyclopropane-1-carboxamide

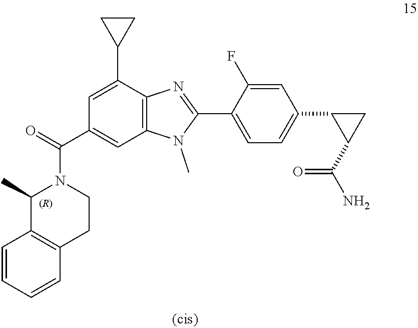

A mixture of compound 14 (0.14 g, 0.27 mmol), HATU (122 mg, 0.32 mmol) and DIPEA (0.18 mL, 1.07 mmol) in DMF (5.5 mL) was stirred at rt for 15 min. Ammonia (30% in H₂O, 0.11 mL, 1.60 mmol) was added and the reaction mixture was stirred at rt for 1 h. The reaction mixture was diluted with EtOAc and H₂O. The layers were separated and the aqueous phase was extracted with EtOAc. The combined organic extracts were washed with water and brine, dried over MgSO₄, filtered and evaporated to dryness. The crude mixture was purified by flash chromatography over silica gel (Puriflash Interchim® 25 g, 30 μm, mobile phase gradient: DCM/MeOH from 100:0 to 97:3). A second purification was performed via achiral SFC (Stationary phase: AMINO 5 μm 150*30 mm, Mobile phase: 75% CO₂, 25% MeOH). The residue (78 mg) was taken up in Et₂O. The solid was filtered off and dried under vacuum to give compound 15 (65 mg, 47%) as a white solid.

8.4 Synthesis of Compounds 16 and 17

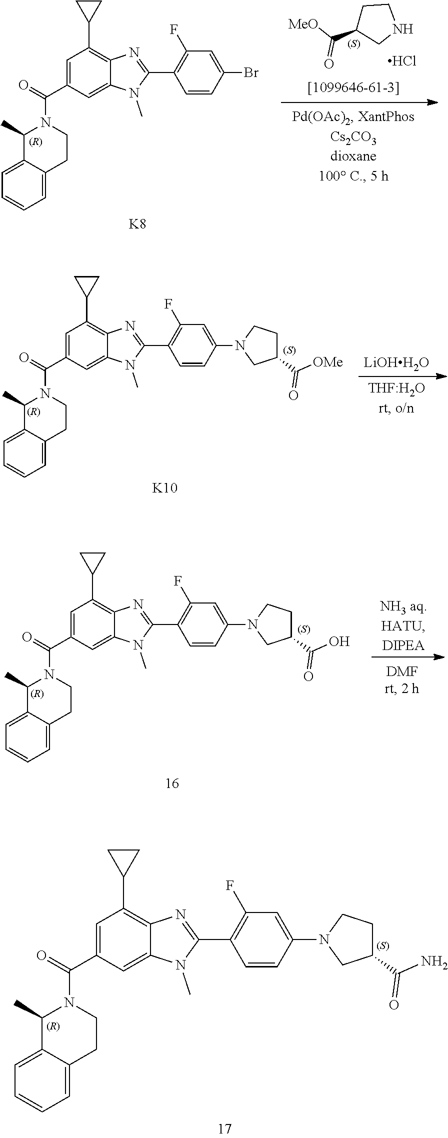

### Intermediate K10

### Methyl (3S)-1-(4-{4-cyclopropyl-1-methyl-6-[(1R)-1-methyl-1,2,3,4-tetrahydroisoquinoline-2-carbonyl]-1H-1,3-benzodiazol-2-yl}-3-fluorophenyl)pyrrolidine-3-carboxylate

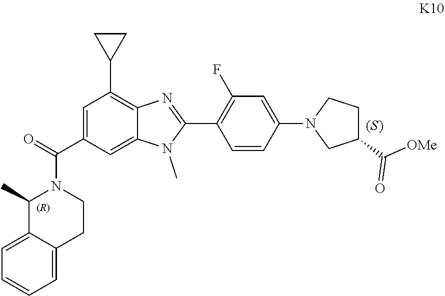

A mixture of intermediate K8 (0.41 g, 0.79 mmol), (S)-methyl pyrrolidine-3-carboxylate hydrochloride [1099646-61-3] (144 mg, 0.87 mmol), cesium carbonate (1.03 g, 3.16 mmol) and XantPhos (45.8 mg, 79.1 μmot) in 1,4-dioxane (7 mL) was purged with nitrogen. Palladium acetate (17.8 mg, 79.1 μmot) was added. The reaction mixture was purged again with nitrogen and stirred at 100° C. for 5 h. The reaction mixture was diluted with EtOAc and H₂O. The layers were separated and the aqueous phase was extracted with EtOAc. The combined organic extracts were washed with brine, dried over MgSO₄, filtered and the solvent was evaporated in vacuo. The crude mixture was purified by flash chromatography over silica gel (Puriflash Interchim® 40 g, 30 μm, mobile phase gradient: DCM/MeOH from 100:0 to 97:3) to afford intermediate K10 (334 mg, 74%) as a white solid.

### Compound 16

### (3S)-1-(4-{4-Cyclopropyl-1-methyl-6-[(1R)-1-methyl-1,2,3,4-tetrahydroisoquinoline-2-carbonyl]-1H-1,3-benzodiazol-2-yl}-3-fluorophenyl)pyrrolidine-3-carboxylic acid

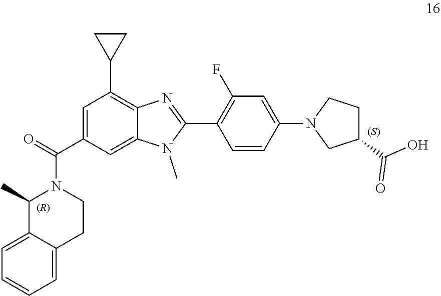

A mixture of intermediate K10 (0.33 g, 582 μmol) and lithium hydroxide monohydrate (147 mg, 3.49 mmol) in THE (15 mL) and H₂O (3 mL) was stirred at rt overnight. An aqueous solution of citric acid (671 mg in 12 mL of H₂O) was added. The layers were separated and the aqueous phase was extracted with EtOAc. The combined organic extracts were washed with brine, dried over MgSO₄, filtered and evaporated to dryness. The residue (0.31 g) was taken up in Et₂O. The solid was filtered off and dried under vacuum to give compound 16 (0.24 g, 74%) as a beige solid.

### Compound 17

### (3S)-1-(4-{4-Cyclopropyl-1-methyl-6-[(1R)-1-methyl-1,2,3,4-tetrahydroisoquinoline-2-carbonyl]-1H-1,3-benzodiazol-2-yl}-3-fluorophenyl)pyrrolidine-3-carboxamide

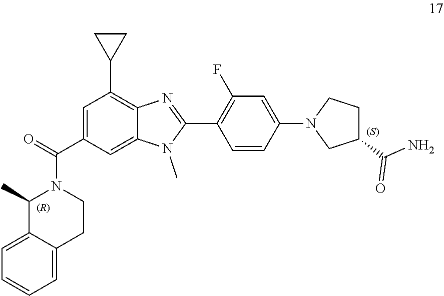

A mixture of compound 16 (0.10 g, 0.18 mmol), HATU (103 mg, 0.27 mmol) and DIPEA (94 μL, 0.54 mmol) in DMF (4 mL) was stirred at rt for 15 min. Ammonia (30% in H₂O, 73 μL, 1.09 mmol) was added and the reaction mixture was stirred at rt for 2 h. The reaction mixture was diluted with EtOAc and H₂O. The layers were separated and the aqueous phase was extracted with EtOAc. The combined organic extracts were washed with H₂O (3 times) and brine, dried over MgSO₄, filtered and evaporated to dryness to give compound 17 (75 mg, 75%) as a white solid.

8.5 Synthesis of compound 18

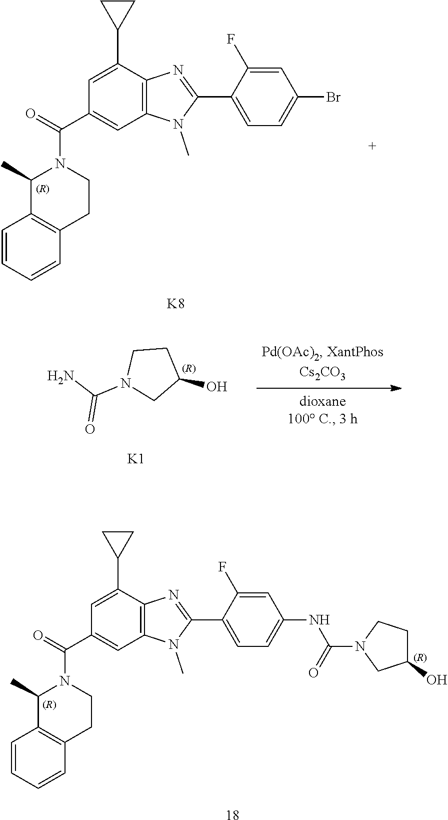

### Compound 18

### (3R)-N-(4-{4-Cyclopropyl-1-methyl-6-[(1R)-1-methyl-1,2,3,4-tetrahydroisoquinoline-2-carbonyl]-1H-1,3-benzodiazol-2-yl}-3-fluorophenyl)-3-hydroxypyrrolidine-1-carboxamide

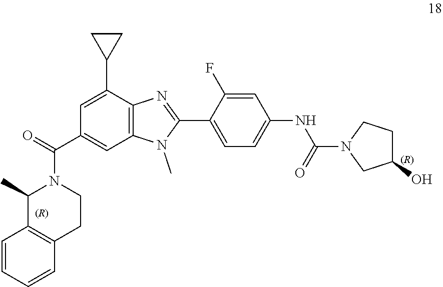

A mixture of intermediate K8 (0.20 g, 0.39 mmol), intermediate K1 (75.3 mg, 0.58 mmol), cesium carbonate (0.63 g, 1.93 mmol) and XantPhos (22.3 mg, 38.6 μmol) in 1,4-dioxane (8 mL) was purged under nitrogen. Palladium acetate (8.66 mg, 38.6 μmol) was added and the reaction mixture was purged again with nitrogen. The reaction mixture was stirred at 100° C. for 3 h. The reaction mixture was diluted with EtOAc and H₂O. The layers were separated. The aqueous phase was extracted with EtOAc. The combined organic extracts were washed with H₂O, dried over MgSO₄, filtered and evaporated to dryness. The crude mixture was purified by flash chromatography over silica gel (Puriflash Interchim® 25 g, 30 μm, mobile phase gradient: DCM/MeOH from 100:0 to 96:4). The residue was taken up in Et₂O. The solid was filtered off and dried under vacuum to give compound 18 (0.12 g, 55%) as a beige solid.

### 9. Synthesis of Indazoles

### 9.1 Synthesis of Compound 19

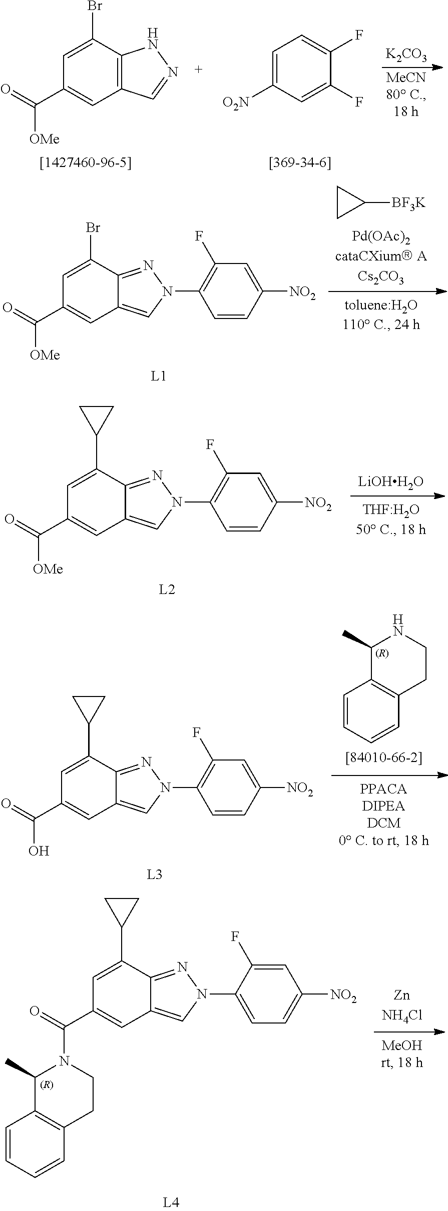

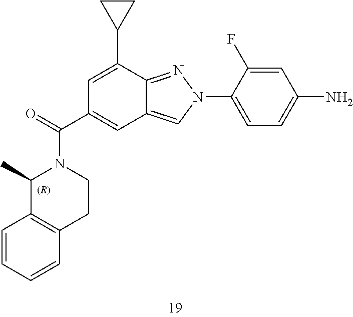

### Intermediate L1

### Methyl 7-bromo-2-(2-fluoro-4-nitrophenyl)-2H-indazole-5-carboxylate

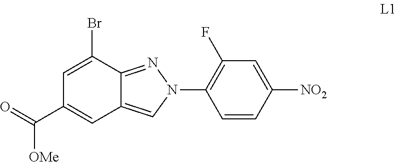 [SMILES: COC(=O)c1cc(Br)c2nn(-c3ccc([N+](=O)[O-])cc3F)cc2c1]

A mixture of methyl 7-bromo-1H-indazole-5-carboxylate [1427460-96-5] (50.0 mg, 196 μmol), 3,4-difluoronitrobenzene [369-34-6] (23.9 μL, 216 μmol) and potassium carbonate (81.3 mg, 0.59 mmol) in MeCN (1.5 mL) was stirred at 80° C. for 18 h. The reaction mixture was diluted with H₂O and EtOAc. The layers were separated and the aqueous phase was extracted with EtOAc. The combined organic extracts were dried over MgSO₄, filtered and concentrated in vacuo to afford intermediate L1 (50 mg, 65%).

### Intermediate L2

### Methyl 7-cyclopropyl-2-(2-fluoro-4-nitrophenyl)-2H-indazole-5-carboxylate

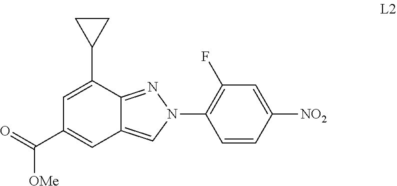 [SMILES: COC(=O)c1cc(C2CC2)c2nn(-c3ccc([N+](=O)[O-])cc3F)cc2c1]

To a degassed mixture of intermediate L1 (1.27, 3.22 mmol), potassium cyclopropyltrifluoroborate [1065010-87-8] (1.19, 8.04 mmol) and cesium carbonate (3.14 g, 9.65 mmol) in H₂O (2.4 mL) and toluene (12 mL) were added Catacxium® A (231 mg, 643 μmol) and palladium acetate (72.2 mg, 0.32 mmol). The reaction mixture was stirred at 110° C. for 24 h. The mixture was diluted with H₂O and EtOAc. The layers were separated and the aqueous phase was extracted with EtOAc (twice). The combined organic extracts were dried over MgSO₄, filtered and the solvent was evaporated in vacuo. The crude mixture was purified by preparative LC (irregular SiOH, 15-40 μm, 80 g GraceResolv™, dry loading (Celite®), mobile phase gradient: heptane/EtOAc from 95:5 to 70:30) to give intermediate L2 (400 mg, 35%) as a yellow solid.

### Intermediate L3

### 7-Cyclopropyl-2-(2-fluoro-4-nitrophenyl)-2H-indazole-5-carboxylic acid

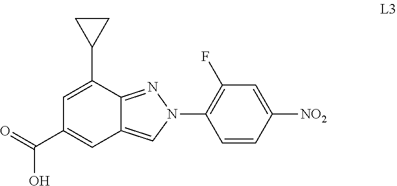 [SMILES: O=C(O)c1cc(C2CC2)c2nn(-c3ccc([N+](=O)[O-])cc3F)cc2c1]

Lithium hydroxide monohydrate (267 mg, 6.35 mmol) was added to a solution of intermediate L2 (410 mg, 1.15 mmol) in THE (34 mL) and H₂O (8.4 mL). The reaction mixture was stirred at 50° C. for 18 h. A 10% aqueous solution of KHSO₄ was added until pH 6 and the aqueous phase was extracted with EtOAc. The combined organic extracts were washed with H₂O, dried over MgSO₄, filtered and evaporated in vacuo to afford intermediate L3 (315 mg, 78%).

### Intermediate L4

### (1R)-2-[7-Cyclopropyl-2-(2-fluoro-4-nitrophenyl)-2H-indazole-5-carbonyl]-1-methyl-1,2,3,4-tetrahydroisoquinoline

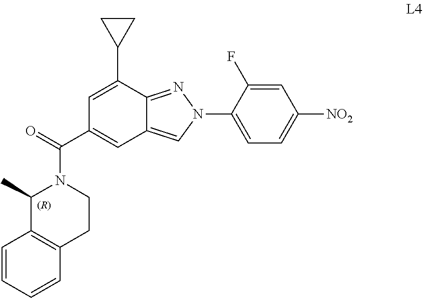

A mixture of intermediate L3 (277 mg, 0.79 mmol), (R)-1-methyl-1,2,3,4-tetrahydroisoquinoline [84010-66-2] (135 mg, 917 μmol) and DIPEA (675 μL, 3.92 mmol) in DCM (2 mL) was stirred at 0° C. PPACA (50 wt. % in EtOAc, 1.20 mL, 2.00 mmol) was added slowly. The reaction mixture was stirred at 0° C. for 10 min and at rt for 18 h. The reaction mixture was diluted with H₂O and EtOAc. The layers were separated and the aqueous phase was extracted with EtOAc. The combined organic extracts were washed with brine, dried over MgSO₄, filtered and evaporated in vacuo. The crude mixture was purified by preparative LC (irregular SiOH, 15-40 μm, 24 g GraceResolv™, mobile phase gradient: heptane/EtOAc from 90:10 to 50:50). The residue was taken up in MeCN and evaporated in vacuo to give intermediate L4 (291 mg, 78%).

### Compound 19

### 4-{7-Cyclopropyl-5-[(1R)-1-methyl-1,2,3,4-tetrahydroisoquinoline-2-carbonyl]-2H-indazol-2-yl}-3-fluoroaniline

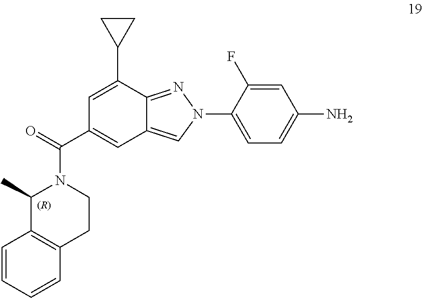

To a solution of intermediate L4 (440 mg, 935 μmol) in MeOH (9.4 mL) were added ammonium chloride (350 mg, 6.55 mmol) and zinc dust (917 mg, 14.0 mmol). The reaction mixture was stirred at rt for 18 h. The reaction mixture was filtered through a pad of Celite®. The filtrate was concentrated in vacuo and diluted with DCM and H₂O. The layers were separated and the organic phase was dried over MgSO₄, filtered and evaporated in vacuo to give compound 19 (362 mg, 88%).

9.2 Synthesis of Compound 20

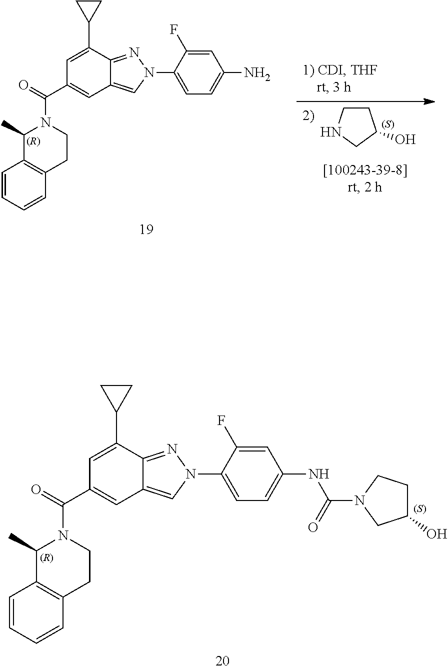

### Compound 20

### (3S)-N-(4-{7-Cyclopropyl-5-[(1R)-1-methyl-1,2,3,4-tetrahydroisoquinoline-2-carbonyl]-2H-indazol-2-yl}-3-fluorophenyl)-3-hydroxypyrrolidine-1-carboxamide

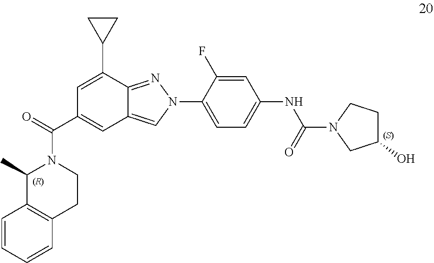

CDI (73.6 mg, 454 μmol) was added to a solution of compound 19 (100 mg, 227 μmol) in THE (850 μL). The reaction mixture was stirred at rt for 3 h. (S)-3-Hydroxypyrrolidine [100243-39-8](23.7 mg, 272 μmol) was added and the reaction mixture was stirred at rt for another 2 h. The reaction mixture was diluted with EtOAc. The organic phase was washed with an aqueous solution of NH₄Cl, dried over MgSO₄, filtered and concentrated to dryness. The crude mixture was combined with another batch (20.0 mg, 45.4 μmol) and purified by preparative LC (spherical C18 25 μm, 40 g YMC-ODS-25, dry loading (Celite®), mobile phase gradient: 0.2% aq. NH₄HCO₃/MeCN from 75:25 to 35:65). The residue (50 mg) was triturated in MTBE. The solid was filtered off and dried under high vacuum at 50° C. overnight to give compound 20 (40 mg, 32%) as a white solid.

### Synthesis of Benzoxazoles

### 9.3 Synthesis of Compound 21

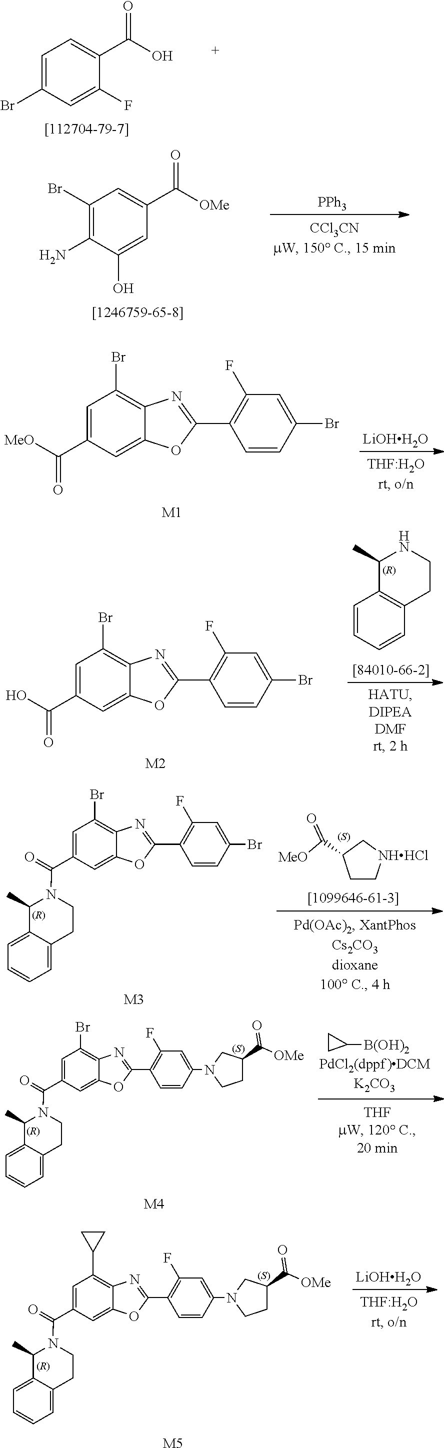

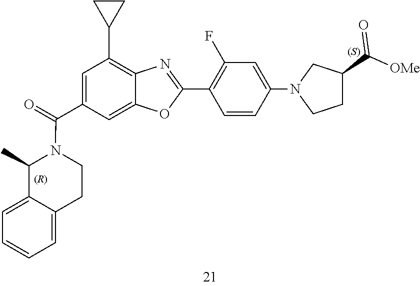

### Intermediate M1

### Methyl 4-bromo-2-(4-bromo-2-fluorophenyl)-1,3-benzoxazole-6-carboxylate

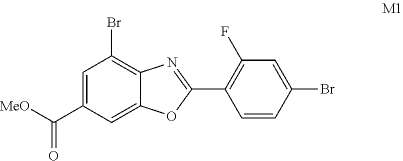 [SMILES: COC(=O)c1cc(Br)c2nc(-c3ccc(Br)cc3F)oc2c1]

To a mixture of methyl 4-amino-3-bromo-5-hydroxybenzoate [1246759-65-8] (1.30 g, 5.28 mmol), 4-bromo-2-fluorobenzoic acid [112704-79-7] (1.74 g, 7.93 mmol) and triphenylphosphine (4.16 g, 15.9 mmol) in MeCN (14 mL) at 0° C. was added trichloroacetonitrile (1.06 mL, 10.6 mmol) dropwise. The reaction mixture was heated at 150° C. using a single mode microwave (Anton Paar Monowave 300) with a power output ranging from 0 to 850 W for 15 min. The reaction mixture was cooled with an ice bath. The precipitate was filtered off and dried under vacuum to afford intermediate M1 (1.68 g, 74%) as a beige solid.

### Intermediate M2

### 4-Bromo-2-(4-bromo-2-fluorophenyl)-1,3-benzoxazole-6-carboxylic acid

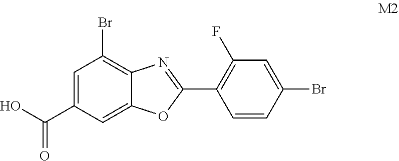 [SMILES: O=C(O)c1cc(Br)c2nc(-c3ccc(Br)cc3F)oc2c1]

A mixture of intermediate M1 (1.00 g, 2.33 mmol) and lithium hydroxide monohydrate (0.44 g, 10.5 mmol) in THE (23 mL) and H₂O (6 mL) was stirred at rt overnight. An aqueous solution of citric acid (2.0 g in 20 mL of H₂O) was added. The precipitate was filtered off, washed with H₂O and Et₂O and dried under vacuum to afford intermediate M2 (0.75 g, used like this in the next step) as a yellow solid.

### Intermediate M3

### (1R)-2-[4-Bromo-2-(4-bromo-2-fluorophenyl)-1,3-benzoxazole-6-carbonyl]-1-methyl-1,2,3,4-tetrahydroisoquinoline

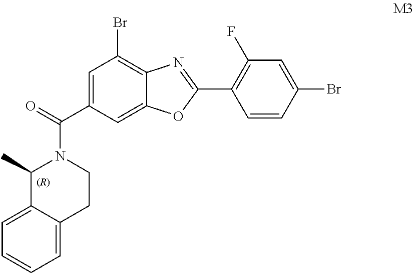

To a mixture of intermediate M2 (0.75 g, 1.82 mmol), (1R)-1-methyl-1,2,3,4-tetrahydroisoquinoline [84010-66-2] (321 mg, 2.18 mmol) and DIPEA (1.27 mL, 7.27 mmol) in DMF (30 mL) was added HATU (829 mg, 2.18 mmol). The reaction mixture was stirred at rt for 2 h. The mixture was poured out slowly into water and the aqueous phase was extracted with EtOAc. The combined organic extracts were washed with brine, dried over MgSO₄, filtered and evaporated to dryness. The crude mixture was purified by flash chromatography over silica gel (Puriflash Interchim® 40 g, 30 μm, Mobile phase gradient: heptane/EtOAc from 100:0 to 70:30) to afford intermediate M3 (0.49 g, 39% over 2 steps) as a white solid.

### Intermediate M4

### Methyl (3S)-1-(4-{4-bromo-6-[(1R)-1-methyl-1,2,3,4-tetrahydroisoquinoline-2-carbonyl]-1,3-benzoxazol-2-yl}-3-fluorophenyl)pyrrolidine-3-carboxylate

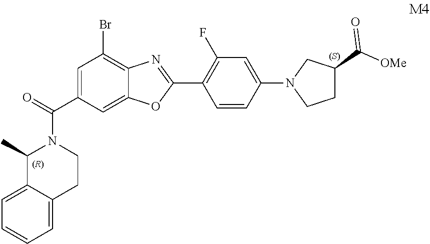

A mixture of intermediate M3 (0.49 g, 0.90 mmol), (S)-methyl pyrrolidine-3-carboxylate hydrochloride [1099646-61-3] (149 mg, 0.90 mmol), cesium carbonate (0.88 g, 2.70 mmol) and XantPhos (52.1 mg, 0.09 mmol) in 1,4-dioxane (11.5 mL) was purged with nitrogen. Palladium acetate (20.2 mg, 0.09 mmol) was added. The reaction mixture was purged again with nitrogen and stirred at 100° C. for 4 h. The reaction mixture was diluted with EtOAc and H₂O. The layers were separated and the aqueous phase was extracted with EtOAc (twice). The combined organic extracts were washed with brine, dried over MgSO₄, filtered and the solvent was evaporated in vacuo. The crude mixture was purified by flash chromatography over silica gel (Puriflash Interchim® 40 g, 30 μm, mobile phase gradient: heptane/EtOAc from 100:0 to 60:40) to afford intermediate M4 (0.26 g, 79%) as a beige solid.

### Intermediate M5

### Methyl (3S)-1-(4-{4-cyclopropyl-6-[(1R)-1-methyl-1,2,3,4-tetrahydroisoquinoline-2-carbonyl]-1,3-benzoxazol-2-yl}-3-fluorophenyl)pyrrolidine-3-carboxylate

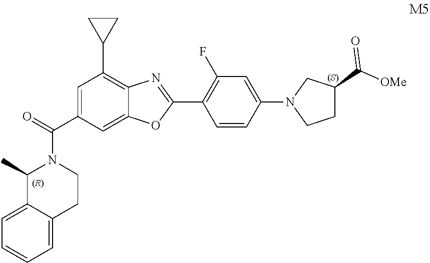

A mixture of intermediate M4 (0.52 g, 0.88 mmol), cyclopropylboronic acid [411235-57-9] (113 mg, 1.32 mmol) and potassium carbonate (607 mg, 4.39 mmol) in THE (10 mL) was purged with nitrogen for 5 min. PdCl₂(dppf).DCM (71.7 mg, 87.8 μmol) was added and the mixture was purged again with nitrogen for 2 min. The reaction mixture was heated at 120° C. using a single mode microwave (Anton Paar Monowave 300) with a power output ranging from 0 to 850 W for 20 min. The reaction mixture was diluted with H₂O and EtOAc. The mixture was filtered through a pad of Celite® and washed with EtOAc. The layers were separated and the organic phase was washed with H₂O, brine, dried over MgSO₄, filtered and evaporated to dryness. The crude mixture was purified by flash chromatography over silica gel (Puriflash Interchim® 25 g, 30 μm, mobile phase gradient: heptane/EtOAc from 100:0 to 60:40) to afford intermediate M5 (0.44 g, 91%) as a beige solid.

### Compound 21

### (3S)-1-(4-{4-Cyclopropyl-6-[(1R)-1-methyl-1,2,3,4-tetrahydroisoquinoline-2-carbonyl]-1,3-benzoxazol-2-yl}-3-fluorophenyl)pyrrolidine-3-carboxylic acid

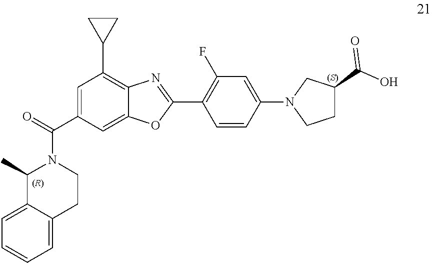

A mixture of intermediate M5 (0.43 g, 0.78 mmol) and lithium hydroxide monohydrate (147 mg, 3.50 mmol) in THE (7 mL) and H₂O (2 mL) was stirred at rt overnight. An aqueous solution of citric acid (0.67 g in 5 mL of H₂O) was added. The precipitate was filtered off, washed with H₂O and dried under vacuum. The residue (0.39 g) was taken up in Et₂O. The solid was filtered off and dried under vacuum to give compound 21 (0.37 g, 88%) as a beige solid.

9.4 Synthesis of Compound 22

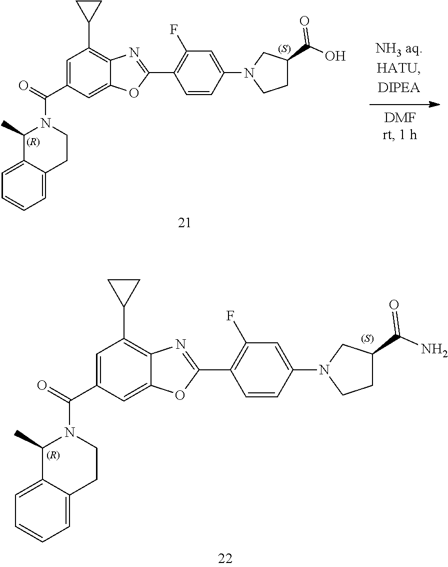

### Compound 22

### (3S)-1-(4-{4-Cyclopropyl-6-[(1R)-1-methyl-1,2,3,4-tetrahydroisoquinoline-2-carbonyl]-1,3-benzoxazol-2-yl}-3-fluorophenyl)pyrrolidine-3-carboxamide

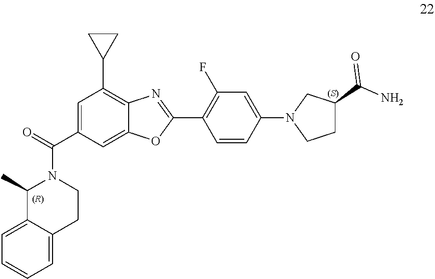

A mixture of compound 21 (0.25 g, 0.46 mmol), HATU (247 mg, 0.65 mmol) and DIPEA (0.32 mL, 1.85 mmol) in DMF (10 mL) was stirred at rt for 15 min. Ammonia (30% in H₂O, 0.19 mL, 2.78 mmol) was added. The reaction mixture was stirred at rt for 1 h. The reaction mixture was diluted with EtOAc and H₂O. The layers were separated and the aqueous phase was extracted with EtOAc. The combined organic extracts were washed with H₂O and brine, dried over MgSO₄, filtered and evaporated to dryness. The crude mixture was purified by flash chromatography over silica gel (Puriflash Interchim® 25 g, 30 μm, mobile phase gradient: DCM/MeOH from 100:0 to 98:2) to give compound 22 (140 mg, 56%) as a beige solid.

9.5 Synthesis of Compound 23

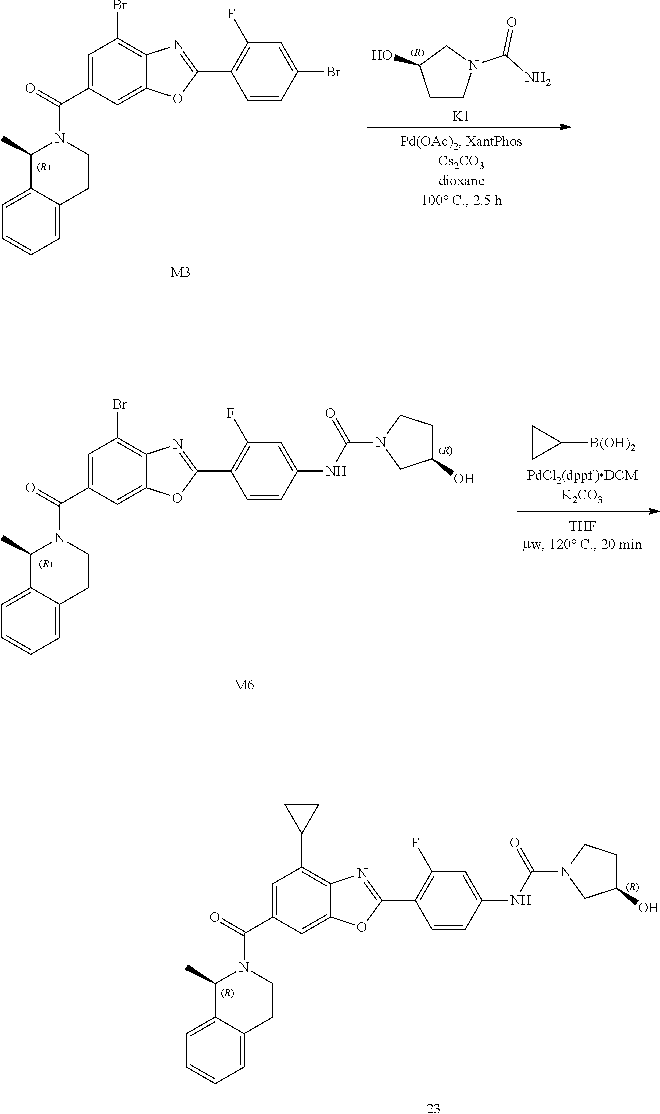

### Intermediate M6

### (R)-N-(4-(4-bromo-6-((R)-1-methyl-1,2,3,4-tetrahydroisoquinoline-2-carbonyl)benzo[d]oxazol-2-yl)-3-fluorophenyl)-3-hydroxypyrrolidine-1-carboxamide

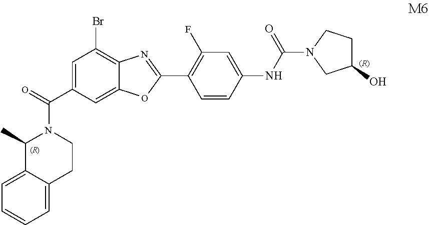

A mixture of intermediate M3 (0.30 g, 0.55 mmol), intermediate K1 (72 mg, 0.55 mmol), cesium carbonate (720 mg, 2.2 mmol) and XantPhos (32 mg, 0.055 mmol) in 1,4-dioxane (9 mL) was purged with nitrogen. Palladium acetate (12 mg, 0.055 mmol) was added. The reaction mixture was purged again with nitrogen and stirred at 100° C. for 4 h. The reaction mixture was diluted with EtOAc and H₂O. The layers were separated and the aqueous phase was extracted with EtOAc (twice). The combined organic extracts were washed with brine, dried over MgSO₄, filtered and the solvent was evaporated in vacuo. The crude mixture was purified by flash chromatography over silica gel (Puriflash Interchim® 40 g, 30 μm, mobile phase gradient: DCM/MeOH from 100:0 to 97:3) to afford intermediate M6 (0.10 g, 30%) as a yellow solid.

### Compound 23

### (R)-N-(4-(4-cyclopropyl-6-((R)-1-methyl-1,2,3,4-tetrahydroisoquinoline-2-carbonyl)benzo[d]oxazol-2-yl)-3-fluorophenyl)-3-hydroxypyrrolidine-1-carboxamide

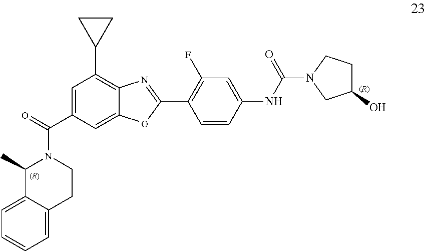

A mixture of intermediate M6 (85 mg, 0.14 mmol), cyclopropylboronic acid [411235-57-9] (18 mg, 0.22 mmol) and potassium carbonate (99 mg, 0.72 mmol) in THE (1.8 mL) was purged with nitrogen for 5 min. PdCl₂(dppf).DCM (12 mg, 14 μmol) was added and the mixture was purged again with nitrogen for 2 min. The reaction mixture was heated at 120° C. using a single mode microwave (Anton Paar Monowave 300) with a power output ranging from 0 to 850 W for 20 min. The reaction mixture was diluted with H₂O and EtOAc. The mixture was filtered through a pad of Celite® and washed with EtOAc. The layers were separated and the organic phase was washed with H₂O, brine, dried over MgSO₄, filtered and evaporated to dryness. The crude mixture was purified by flash chromatography over silica gel (Puriflash Interchim® 12 g, 30 μm, mobile phase gradient: DCM/MeOH from 100:0 to 98:2) to afford after evaporation of the pure fraction a residue which was triturated in Et₂O to give after filtration compound 23 (52 mg, 65%) as a beige solid.

### 10. Synthesis of Benzothiazoles

### 10.1 Synthesis of Compound 24

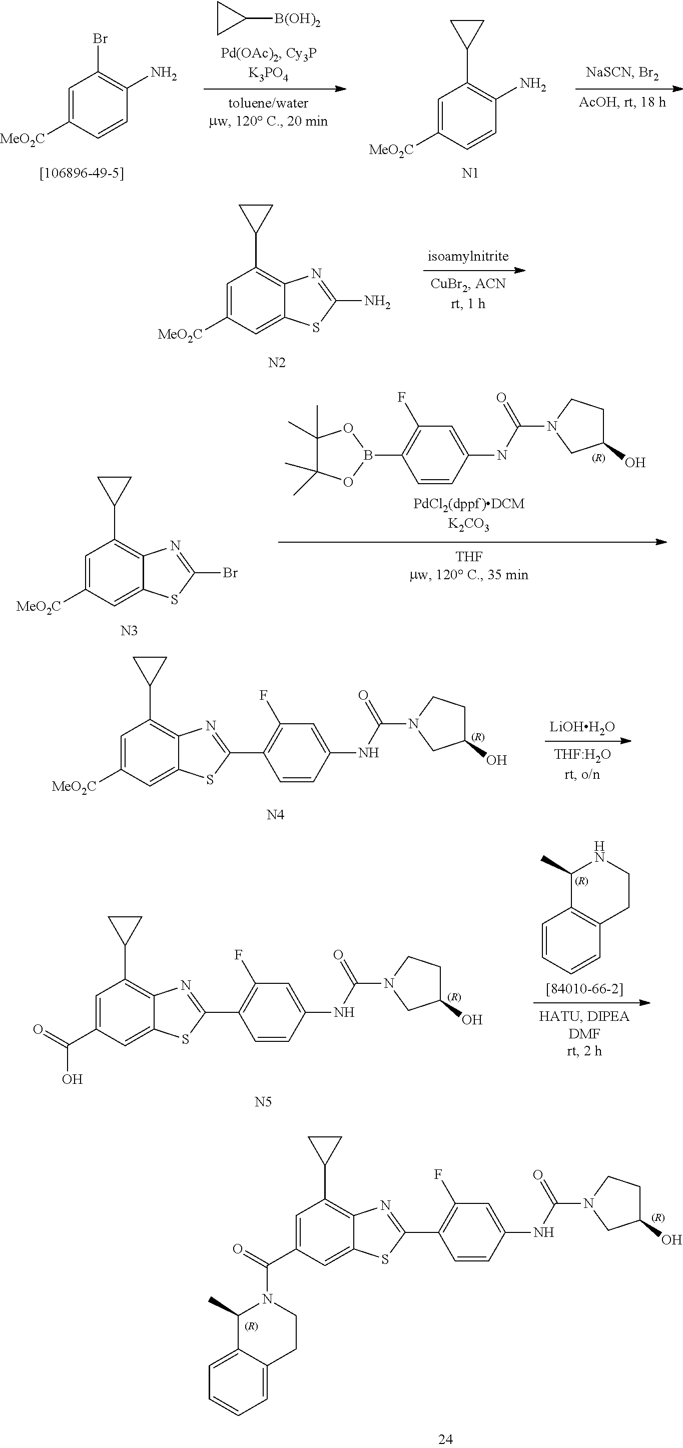

### Intermediate N1

### Methyl 4-amino-3-cyclopropylbenzoate

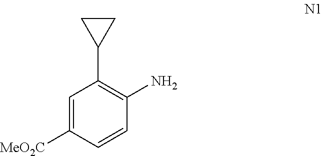 [SMILES: COC(=O)c1ccc(N)c(C2CC2)c1]

To a solution of 4-amino-3-bromobenzoic acid methyl ester (1.0 g, 4.4 mmol) in toluene (15 mL), were added cyclopropylboronic acid (0.56 g, 6.5 mmol), K₃PO₄ (2.8 g, 13 mol), tricyclohexylphoshine (0.12 g, 0.44 mmol) and H₂O (2 mL). The reaction mixture was purged again for 2 min then was heated at 120° C. using a single mode microwave (Anton Paar Monowave 300) with a power output ranging from 0 to 850 W for 45 min. The reaction mixture was filtered through a pad of Celite® and washed with EtOAc and H₂O. The filtrate obtained was extracted with EtOAc and the organic layer was washed with brine, dried over MgSO₄, filtered and evaporated till dryness. The residue was purified by preparative LC (regular SiOH 30 μm, 40 g Interchim®, mobile phase gradient: from heptane/EtOAc from 100:0 to 70:30). The pure fractions were combined and evaporated under vacuum to give intermediate N1 as a yellow oil (0.77 g, 93%).

### Intermediate N2

### Methyl 2-amino-4-cyclopropylbenzo[d]thiazole-6-carboxylate

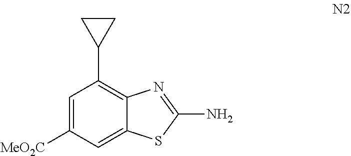 [SMILES: COC(=O)c1cc(C2CC2)c2nc(N)sc2c1]

To a mixture of sodium thiocyanate (1.3 g, 16 mmol) in HOAc (15 mL) at 0° C. was added dropwise a solution of intermediate N1 (0.77 g, 4.0 mmol) in HOAc (15 mL) followed by the addition of Bromine (0.25 mL, 4.83 mmol) dropwise. The reaction mixture was stirred at rt for overnight. Water (50 mL) was added and stirred at rt for 2 h. The yellow precipitate was filtered. The solid obtained was diluted in DCM/MeOH (9/1) and basified with NH₃ aq until pH 8. The resulting mixture was filtered through a short pad of Celite®. The organic layer was evaporated till dryness and the residue was taken up in MeOH and stirred overnight at rt. The solid was filtered and rinsed with MeOH and dried in vacuo to give intermediate N₂ (0.55 g, 55%) as a yellow solid.

### Intermediate N3

### Methyl 2-bromo-4-cyclopropylbenzo[d]thiazole-6-carboxylate

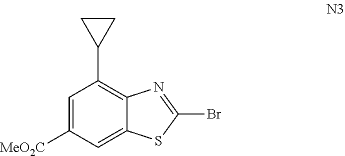 [SMILES: COC(=O)c1cc(C2CC2)c2nc(Br)sc2c1]

To a solution of isoamyl nitrite (0.43 mL, 3.06 mmol) and CuBr₂ (0.55 g, 2.45 mmol) in ACN (8 mL) under N₂ and cooled in ice bath was added dropwise a solution of intermediate N₂ (0.55 g, 2.04 mmol) in ACN (2.1 mL). The reaction mixture was stirred at rt for 1 h. H₂O and HCl (1N) were added. The mixture was extracted twice with DCM. The organic layers were dried over MgSO₄, filtered and evaporated till dryness. The residue was purified by flash chromatography over silica gel (Puriflash Interchim® 40 g, 30 μm, dry loading Celite®, Mobile phase gradient: Hept/EtOAc, from 100/0 to 80/20). The pure fractions were collected and evaporated to dryness to give intermediate N₃ (0.41 g, 64%) as a white powder.

### Intermediate N4

### Methyl (R)-4-cyclopropyl-2-(2-fluoro-4-(3-hydroxypyrrolidine-1-carboxamido)phenyl) benzo[d]thiazole-6-carboxylate

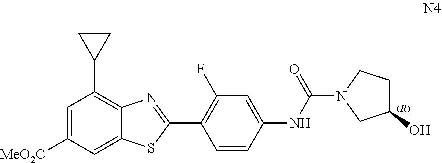

The solution of intermediate N₃ (0.30 g, 0.96 mmol), (R)-N-(3-fluoro-4-(4,4,5,5-tetramethyl-1,3,2-dioxaborolan-2-yl)phenyl)-3-hydroxypyrrolidine-1-carboxamide (1.0 g, 1.15 mmol) and K₂CO₃ 2M in water (1.44 mL, 2.88 mmol) in THF (9.6 mL) was purged under N₂ for 5 min then to this solution was added PdCl₂dppf·DCM (79 mg, 0.10 mmol). The reaction mixture was purged again for 2 min then was heated at 120° C. using a single mode microwave (Anton Paar Monowave 300) with a power output ranging from 0 to 850 W for 35 min. H₂O and EtOAc were added and separated. The aqueous layer was extracted with EtOAc. the combined organic layers were washed with H2o, brine, dried over MgSO₄, filtered and evaporated till dryness. The crude was purified by flash chromatography over silica gel (Puriflash Interchim® 25 g, 30 μm, dry loading (Celite®), Mobile phase gradient: Hept/EtOAc, from 80/20 to 0/100). The pure fractions were collected and evaporated to dryness to give intermediate N₄ (0.17 g, 34%) as a beige solid.

### Intermediate N5

### (R)-4-cyclopropyl-2-(2-fluoro-4-(3-hydroxypyrrolidine-1-carboxamido)phenyl)benzo[d]-thiazole-6-carboxylic acid

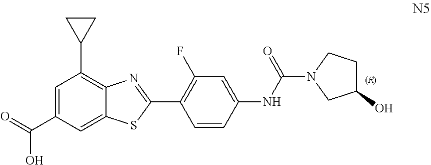

A mixture of intermediate N₄ (0.17 g, 0.29 mmol) and LiOH·H₂O (86 mg, 2.04 mmol) in THF (7 mL) and water (1.5 mL) was stirred and refluxed for 5 h. An aqueous solution of citric acid was added (390 mg in 10 mL of H₂O). The mixture was extracted with EtOAc, washed with brine, dried over MgSO₄, filtered and evaporated to dryness to afford intermediate N₅ (0.14 g, quant.) as a yellow solid.

### Compound 24

### (R)-N-(4-(4-cyclopropyl-6-((R)-1-methyl-1,2,3,4-tetrahydroisoquinoline-2-carbonyl)benzo[d]thiazol-2-yl)-3-fluorophenyl)-3-hydroxypyrrolidine-1-carboxamide

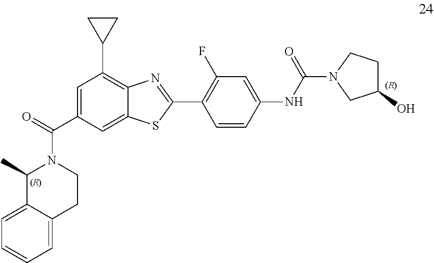

To a mixture of intermediate N5 (0.14 g, 0.30 mmol), (1R)-1-methyl-1,2,3,4-tetrahydroisoquinoline [84010-66-2] (56 mg, 0.38 mmol) and DIPEA (0.16 mL, 0.89 mmol) in DMF (3.7 mL) was added HATU (0.12 g, 0.32 mmol). The reaction mixture was stirred at rt for 2 h. The mixture was poured out slowly into water and the aqueous phase was extracted with EtOAc. The combined organic extracts were washed with brine, dried over MgSO₄, filtered and evaporated to dryness. The crude mixture was purified by flash chromatography over silica gel (Puriflash Interchim® 25 g, 30 μm, Mobile phase gradient: DCM/MeOH from 100:0 to 98:2) to afford after trituration in Et₂O, filtration, compound 24 (90 mg, 53%) as a beige solid.

### 11. Synthesis of Indazole

### 11.1 Synthesis of Compound 25

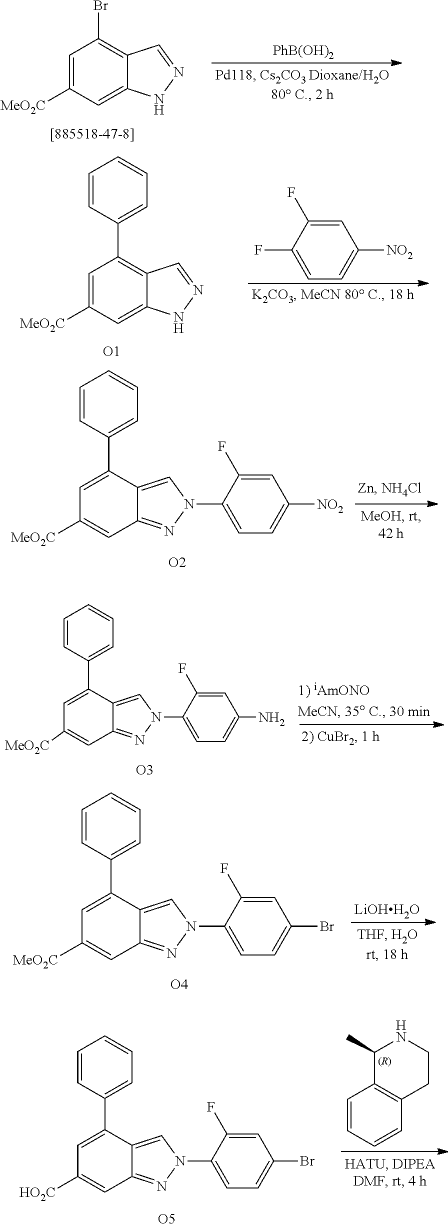

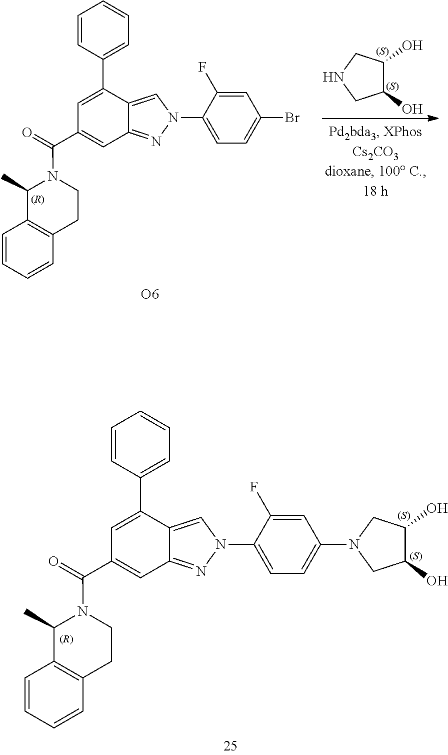

### Intermediate O1

### Methyl 4-phenyl-1H-indazole-6-carboxylate

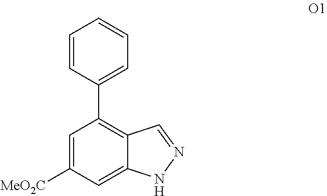 [SMILES: COC(=O)c1cc(-c2ccccc2)c2cn[nH]c2c1]

A mixture of methyl 4-bromo-1H-indazole-6-carboxylate (1 g, 3.92 mmol), phenylboronic acid (1.2 g, 5.88 mmol) and Cs₂CO₃ (3.8 g, 11.8 mmol) in H₂O (5 mL) and Dioxane (15 mL) was purged with N₂ then Pd118 (256 mg, 0.392 mmol) was added and the mixture was purged with N₂ again. The resulting mixture was stirred at 80° C. for 2 h. The mixture was cooled down to rt then EtOAc and water were added. The organic layer was washed with brine (once), dried over MgSO₄, filtered, evaporated and purified by preparative LC (Irregular SiOH, 15-40 μm, 120 g GraceResolv®, mobile phase gradient: from DCM/MeOH 100/0 to 95/5). The fractions containing product were evaporated to give intermediate O1 (830 mg, 84%).

### Intermediate O2

### Methyl 2-(2-fluoro-4-nitrophenyl)-4-phenyl-2H-indazole-6-carboxylate

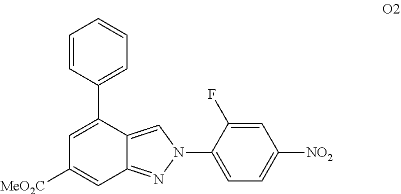 [SMILES: COC(=O)c1cc(-c2ccccc2)c2cn(-c3ccc([N+](=O)[O-])cc3F)nc2c1]

Intermediate O1 (630 mg, 2.50 mmol), 3.4-difluoronitrobenzene (304 μL, 2.75 mmol), K₂CO₃ (1.04 g, 7.49 mmol) in MeCN (19 mL) were stirred at 80° C. for 18 h. The mixture was cooled down to rt then water and EtOAc were added to the reaction mixture. The layers were separated. The aqueous layer was extracted twice with EtOAc. The combined organic layers were washed with brine, dried over MgSO₄, filtered, evaporated and purified by preparative LC (irregular SiOH 15-40 μm, 12 g GraceResolv®, mobile phase: DCM 100%). The fractions containing product were evaporated to give intermediate O2 as yellow foam (150 mg, 15%).

### Intermediate O3

### Methyl 2-(4-amino-2-fluorophenyl)-4-phenyl-2H-indazole-6-carboxylate

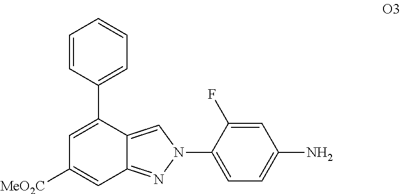 [SMILES: COC(=O)c1cc(-c2ccccc2)c2cn(-c3ccc(N)cc3F)nc2c1]

A mixture of intermediate O2 (140 mg, 0.358 mmol), MeOH (3.5 mL), zinc dust (351 mg, 5.37 mmol) and NH₄Cl (134 mg, 2.50 mmol) was stirred at rt for 18 h. Zinc dust (351 mg, 5.37 mmol) and NH₄Cl (134 mg, 2.50 mmol) were added and the mixture was stirred at rt for 24 h. The mixture was filtered on a Celite® pad, DCM and saturated aqueous solution of NaHCO₃ were added. The mixture as stirred at rt for 2 h. The layer was separated. The organic layer was dried over MgSO₄, filtered and evaporated to give intermediate O3 as yellow solid (129 mg, Quant.).

### Intermediate O4

### Methyl 2-(4-bromo-2-fluorophenyl)-4-phenyl-2H-indazole-6-carboxylate

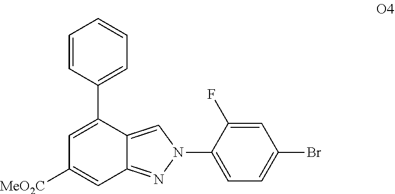 [SMILES: COC(=O)c1cc(-c2ccccc2)c2cn(-c3ccc(Br)cc3F)nc2c1]

To a solution of intermediate O3 (129 mg, 0.357 mmol) in MeCN (2.2 mL) was added isoamylnitrite (72 μL, 0.54 mmol) dropwise then warmed at 35° C. and stirred for 30 minutes. The reaction mixture was then allowed to cool down to room temperature and purged with nitrogen. CuBr₂ (100 mg, 0.446 mmol) was added in one portion. The reaction mixture was purged again with nitrogen, warmed to 35° C. and stirred for 1 hour. The reaction mixture was cooled down to room temperature, diluted with EtOAc and water. The organic layer was separated, washed with water, then with brine, dried over MgSO₄, filtered and evaporated and purified by preparative LC (regular SiOH 40 μm, 24 g Buchi®, mobile phase gradient: from heptane/EtOAc 100:0 to 20:80). The fractions containing product were combined and evaporated under vacuum to give intermediate O4 as a white solid (26 mg, 17%).

### Intermediate O5

### 2-(4-bromo-2-fluorophenyl)-4-phenyl-2H-indazole-6-carboxylic acid

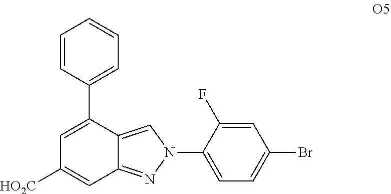 [SMILES: O=C(O)c1cc(-c2ccccc2)c2cn(-c3ccc(Br)cc3F)nc2c1]

A mixture of intermediate O4 (26 mg; 0.061 mmol) and lithium hydroxide monohydrate (5 mg; 0.12 mmol) in THE (1.4 mL) and H₂O (0.1 mL) was stirred at room temperature for 18 h. EtOAc and 10% aq. KHSO₄ were added to the mixture and an extraction was performed. The aqueous layer was extracted with EtOAc. The organic layer was combined, washed with brine, dried over MgSO₄, filtered and evaporated to give intermediate O5 as white solid (26 mg, quant.).

### Intermediate O6

### (R)-(2-(4-bromo-2-fluorophenyl)-4-phenyl-2H-indazol-6-yl)(1-methyl-3,4-dihydroisoquinolin-2(1H)-yl)methanone

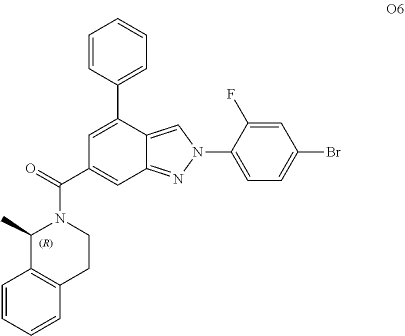

A mixture of intermediate O5 (26 mg, 0.0632 mmol), (1R)-methyl-(1,2,3,4)-tetrahydroisoquinoline (10 mg, 0.0695 mmol), HATU (36 mg, 0.0948 mmol) and DIPEA (33 μL, 0.190 mmol) in DMF (0.5 mL) was stirred at rt for 4 h. Water and EtOAc were added to the reaction mixture. The layers were separated. The aqueous layer was extracted twice with EtOAc. The combined organic layers were washed with brine, dried over MgSO₄, filtered, evaporated and purified by preparative LC (irregular SiOH 15-40 μm, 12 g GraceResolv®, mobile phase gradient: from Heptane/EtOAc 75/25 to 0/100). The fractions containing product were evaporated to give intermediate O6 as a white solid (23 mg, 67%).

### Compound 25

### (2-(4-((3S,4S)-3,4-dihydroxypyrrolidin-1-yl)-2-fluorophenyl)-4-phenyl-2H-indazol-6-yl)((R)-1-methyl-3,4-dihydroisoquinolin-2 (1H)-yl)methanone

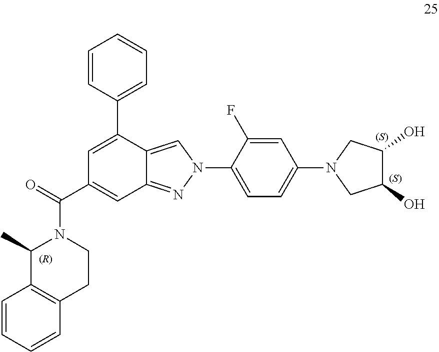

A mixture of intermediate O6 (23 mg, 0.0426 mmol), (3S,4S)-Pyrrolidine-3,4-diol (5 mg, 0.051 mmol) and Cs₂CO₃ (69 mg, 0.21 mmol) was charged in a sealed tube and purged with N₂. Dioxane (0.5 mL) was added and the mixture was degassed with N₂, then XPhos (4 mg, 8.51 μmol) and Pd₂(dba)₃ (2.0 mg, 2.1 μmol) were added. The reaction mixture was purged with N₂ then was stirred and heated at 100° C. for 18 h. The mixture was cooled down to rt then water and EtOAc were added. The aqueous layer was extracted with EtOAc, the combined organic layers were dried over MgSO₄, filtered, concentrated in vacuo and purified by preparative LC (irregular SiOH 15-40 μm, 40 g Buchi®, mobile phase gradient: from DCM/MeOH 100:0 to 88:12). The fractions containing product were evaporated then purified by preparative LC (spherical C18 25 μm, 40 g YMC-ODS-25, mobile phase gradient 0.2% aq. NH⁴⁺HCO₃/MeCN from 75:25 to 0:100). The fractions containing product were freeze-dried to give compound 25 as a pale yellow solid (9 mg, 38%).

### C. Compound Identification

### ¹H-NMR

¹H-NMR spectra were recorded on a Bruker Avance DRX 400 spectrometer using internal deuterium lock and equipped with reverse double-resonance (¹H, ¹³C, SEI) probe head with z gradients and operating at 400 MHz for proton and 100 MHz for carbon and a Bruker Avance 500 MHz spectrometer equipped with a Bruker 5 mm BBFO probe head with z gradients and operating at 500 MHz for proton and 125 MHz for carbon.

NMR spectra were recorded at ambient temperature unless otherwise stated.

Data are reported as follow: chemical shift in parts per million (ppm) relative to TMS (8=0 ppm) which was used as internal standard, integration, multiplicity (s=singulet, d=doublet, t=triplet, q=quartet, quin=quintuplet, sex=sextuplet, m=multiplet, b=broad, or a combination of these), coupling constant(s) J in Hertz (Hz).

### Compound 1

Major rotamer (65%)¹H NMR (500 MHz, DMSO-d₆) δ ppm 8.79 (s, 1H), 8.13 (t, J=8.5 Hz, 1H), 7.83 (br d, J=13.2 Hz, 1H), 7.52-7.62 (m, 2H), 7.03-7.34 (m, 4H), 5.56-5.64 (m, 1H), 4.93-5.02 (m, 1H), 4.32 (br s, 1H), 3.86 (br d, J=10.1 Hz, 1H), 3.31-3.55 (m, 5H), 2.81-3.22 (m, 3H), 2.74 (br d, J=16.1 Hz, 1H), 1.95 (br dd, J=8.4, 4.3 Hz, 1H), 1.84 (br s, 1H), 1.49-1.60 (m, 3H), 1.30-1.42 (m, 3H).

Minor rotamer (35%)¹H NMR (500 MHz, DMSO-d₆) δ ppm 8.79 (s, 1H), 8.13 (t, J=8.5 Hz, 1H), 7.83 (br d, J=13.2 Hz, 1H), 7.52-7.62 (m, 2H), 7.03-7.34 (m, 4H), 4.93-5.02 (m, 2H), 4.58 (br dd, J=13.1, 4.6 Hz, 1H), 4.32 (br s, 1H), 3.31-3.55 (m, 5H), 2.81-3.22 (m, 4H), 1.95 (br dd, J=8.4, 4.3 Hz, 1H), 1.84 (br s, 1H), 1.49-1.60 (m, 3H), 1.30-1.42 (m, 3H).

### Compound 2

Major rotamer (65%)¹H NMR (500 MHz, DMSO-d₆) δ ppm 7.99 (t, J=8.7 Hz, 1H), 7.04-7.32 (m, 5H), 6.58 (br d, J=8.8 Hz, 1H), 6.51 (br d, J=14.8 Hz, 1H), 5.59 (q, J=6.8 Hz, 1H), 5.22 (d, J=2.5 Hz, 2H), 4.09 (br s, 2H), 3.83 (br dd, J=13.4, 3.9 Hz, 1H), 3.58 (dd, J=10.7, 3.5 Hz, 2H), 3.37-3.51 (m, 1H), 3.22-3.27 (m, 2H), 2.80-3.07 (m, 1H), 2.64-2.77 (m, 1H), 2.37-2.47 (m, 1H), 1.48-1.58 (m, 3H), 1.19-1.31 (m, 4H).

Minor rotamer (35%)¹H NMR (500 MHz, DMSO-d₆) δ ppm 7.99 (t, J=8.7 Hz, 1H), 7.04-7.32 (m, 5H), 6.58 (br d, J=8.8 Hz, 1H), 6.51 (br d, J=14.8 Hz, 1H), 5.22 (d, J=2.5 Hz, 2H), 4.97 (br d, J=6.6 Hz, 1H), 4.56 (br dd, J=12.8, 5.2 Hz, 1H), 4.09 (br s, 2H), 3.58 (dd, J=10.7, 3.5 Hz, 2H), 3.22-3.27 (m, 3H), 2.80-3.07 (m, 2H), 2.37-2.47 (m, 1H), 1.48-1.58 (m, 3H), 1.19-1.31 (m, 4H).

### Compound 3

¹H NMR (500 MHz, DMSO-d₆, 77° C.) 8 ppm 7.75 (t, J=8.8 Hz, 1H), 7.45 (s, 1H), 7.13-7.25 (m, 4H), 7.10 (s, 1H), 6.97 (d, J=3.2 Hz, 1H), 6.50 (dd, J=8.7, 2.4 Hz, 1H), 6.44 (dd, J=14.7, 2.0 Hz, 1H), 5.24-5.48 (m, 1H), 4.89-5.01 (m, 2H), 4.09 (br s, 2H), 3.99 (br s, 1H), 3.56 (dd, J=10.6, 3.9 Hz, 2H), 3.37 (br t, J=11.2 Hz, 1H), 3.18 (d, J=10.4 Hz, 2H), 2.91-3.01 (m, 3H), 2.75 (br d, J=16.7 Hz, 1H), 1.50 (d, J=6.9 Hz, 3H), 1.36 (t, J=7.6 Hz, 3H).

### Compound 4

Major rotamer (65%)¹H NMR (400 MHz, DMSO-d₆) δ ppm 7.87-7.98 (m, 1H) 7.68-7.80 (m, 1H) 6.99-7.37 (m, 5H) 6.41-6.57 (m, 2H) 5.59 (q, J=6.8 Hz, 1H) 5.21 (d, J=3.3 Hz, 2H) 4.07 (br s, 2H) 3.72-3.83 (m, 1H) 3.54 (dd, J=10.5, 3.5 Hz, 2H) 3.39-3.49 (m, 1H) 3.18 (d, J=10.8 Hz, 2H) 2.67-3.09 (m, 3H) 1.49-1.60 (m, 3H) 1.13-1.26 (m, 2H) 0.93-1.04 (m, 2H).

Minor rotamer (35%)¹H NMR (400 MHz, DMSO-d₆) δ ppm 7.87-7.98 (m, 1H) 7.68-7.80 (m, 1H) 6.99-7.37 (m, 5H) 6.41-6.57 (m, 2H) 5.21 (d, J=3.3 Hz, 2H) 4.84-5.00 (m, 1H) 4.51-4.62 (m, 1H) 4.07 (br s, 2H) 3.54 (dd, J=10.5, 3.5 Hz, 2H) 3.23-3.29 (m, 1H) 3.18 (d, J=10.8 Hz, 2H) 2.67-3.09 (m, 3H) 1.49-1.60 (m, 3H) 1.13-1.26 (m, 2H) 0.93-1.04 (m, 2H).

### Compound 5

Major rotamer (65%)¹H NMR (400 MHz, DMSO-d₆) δ ppm 8.06-8.22 (m, 1H) 6.94-7.35 (m, 5H) 6.43-6.64 (m, 2H) 5.57 (q, J=6.8 Hz, 1H) 5.19 (d, J=3.1 Hz, 2H) 3.96-4.12 (m, 2H) 3.72 (br dd, J=13.4, 4.0 Hz, 1H) 3.47-3.60 (m, 2H) 3.35-3.45 (m, 1H) 3.18 (br d, J=11.2 Hz, 2H) 2.64-3.03 (m, 2H) 2.05-2.18 (m, 1H) 1.42-1.56 (m, 3H) 1.08-1.24 (m, 2H) 0.95-1.08 (m, 2H).

Minor rotamer (35%)¹H NMR (400 MHz, DMSO-d₆) δ ppm 8.06-8.22 (m, 1H) 6.94-7.35 (m, 5H) 6.43-6.64 (m, 2H) 5.19 (d, J=3.1 Hz, 2H) 4.82-4.94 (m, 1H) 4.48-4.61 (m, 1H) 3.96-4.12 (m, 2H) 3.47-3.60 (m, 2H) 3.35-3.45 (m, 1H) 3.18 (br d, J=11.2 Hz, 2H) 2.64-3.03 (m, 2H) 2.05-2.18 (m, 1H) 1.42-1.56 (m, 3H) 1.08-1.24 (m, 2H) 0.95-1.08 (m, 2H).

### Compound 6

Major rotamer (65%)¹H NMR (400 MHz, DMSO-d₆) δ ppm 8.67 (s, 1H) 7.95 (t, J=8.8 Hz, 1H) 7.80 (br d, J=14.8 Hz, 1H) 7.54 (br d, J=8.8 Hz, 1H) 7.44 (s, 1H) 6.98-7.37 (m, 5H) 5.53-5.68 (m, 1H) 4.91-5.06 (m, 1H) 4.32 (br s, 1H) 3.82 (br dd, J=13.1, 3.7 Hz, 1H) 3.34-3.58 (m, 5H) 2.69-3.12 (m, 4H) 1.77-2.03 (m, 2H) 1.50-1.61 (m, 3H) 1.33-1.45 (m, 3H).

Minor rotamer (35%)¹H NMR (400 MHz, DMSO-d₆) δ ppm 8.67 (s, 1H) 7.95 (t, J=8.8 Hz, 1H) 7.80 (br d, J=14.8 Hz, 1H) 7.54 (br d, J=8.8 Hz, 1H) 7.39 (s, 1H) 6.98-7.37 (m, 5H) 4.91-5.06 (m, 2H) 4.55-4.68 (m, 1H) 4.32 (br s, 1H) 3.34-3.58 (m, 4H) 3.22-3.28 (m, 1H) 2.69-3.12 (m, 4H) 1.77-2.03 (m, 2H) 1.50-1.61 (m, 3H) 1.33-1.45 (m, 3H).

### Compound 7

Major rotamer (65%)¹H NMR (400 MHz, DMSO-d₆) δ ppm 8.98-9.10 (m, 1H) 8.66 (s, 1H) 7.75-7.93 (m, 2H) 7.56 (br d, J=8.9 Hz, 1H) 7.02-7.38 (m, 5H) 5.57-5.68 (m, 1H) 4.95-5.07 (m, 1H) 4.32 (br s, 1H) 3.83 (br dd, J=13.4, 3.8 Hz, 1H) 3.34-3.57 (m, 5H) 2.68-3.11 (m, 2H) 2.42-2.48 (m, 1H) 1.89-2.03 (m, 1H) 1.83 (m, 1.7 Hz, 1H) 1.53 (d, J=6.7 Hz, 3H) 1.29-1.37 (m, 2H) 1.14-1.22 (m, 2H).

Minor rotamer (35%)¹H NMR (400 MHz, DMSO-d₆) δ ppm 8.98-9.10 (m, 1H) 8.66 (s, 1H) 7.75-7.93 (m, 2H) 7.56 (br d, J=8.9 Hz, 1H) 7.02-7.38 (m, 5H) 4.95-5.07 (m, 2H) 4.55-4.65 (m, 1H) 4.32 (br s, 1H) 3.34-3.57 (m, 4H) 3.20-3.29 (m, 1H) 2.68-3.11 (m, 2H) 2.42-2.48 (m, 1H) 1.89-2.03 (m, 1H) 1.83 (m, 1.7 Hz, 1H) 1.53 (d, J=6.7 Hz, 3H) 1.29-1.37 (m, 2H) 1.14-1.22 (m, 2H).

### Compound 8

Major rotamer (70%)¹H NMR (500 MHz, DMSO-d₆) δ ppm 11.61 (s, 1H), 7.83 (t, J=8.2 Hz, 1H), 7.07-7.29 (m, 8H), 6.59 (br s, 1H), 5.56 (br s, 1H), 3.78 (br s, 1H), 3.17-3.52 (m, 2H), 2.87-3.03 (m, 1H), 2.68-2.84 (m, 1H), 2.24-2.33 (m, 1H), 1.88-1.96 (m, 1H), 1.50 (d, J=6.9 Hz, 3H), 1.37-1.53 (m, 3H), 0.98-1.07 (m, 2H), 0.80 (br s, 2H).

Minor rotamer (30%)¹H NMR (500 MHz, DMSO-d₆) δ ppm 11.61 (s, 1H), 7.83 (t, J=8.2 Hz, 1H), 7.07-7.29 (m, 8H), 6.59 (br s, 1H), 4.91 (br s, 1H), 4.52 (br s, 1H), 3.17-3.52 (m, 2H), 2.87-3.03 (m, 1H), 2.68-2.84 (m, 1H), 2.24-2.33 (m, 1H), 1.88-1.96 (m, 1H), 1.50 (d, J=6.9 Hz, 3H), 1.37-1.53 (m, 3H), 0.98-1.07 (m, 2H), 0.80 (br s, 2H).

### Compound 9

Major rotamer (70%)¹H NMR (500 MHz, DMSO-d₆) δ ppm 12.42 (br s, 1H), 7.13-7.51 (m, 8H), 6.76 (s, 1H), 6.64 (br s, 1H), 5.58 (br s, 1H), 3.76 (br s, 1H), 3.62 (s, 3H), 3.19-3.46 (m, 2H), 2.92-3.04 (m, 1H), 2.74 (br s, 1H), 2.24-2.33 (m, 1H), 1.92-2.00 (m, 1H), 1.52 (d, J=6.6 Hz, 3H), 1.43-1.54 (m, 2H), 0.98-1.06 (m, 2H), 0.81 (br s, 2H).

Minor rotamer (30%)¹H NMR (500 MHz, DMSO-d₆) δ ppm 12.42 (br s, 1H), 7.13-7.51 (m, 8H), 6.76 (s, 1H), 6.64 (br s, 1H), 4.87 (br s, 1H), 4.54 (br s, 1H), 3.62 (s, 3H), 3.19-3.46 (m, 2H), 2.92-3.04 (m, 1H), 2.74 (br s, 1H), 2.24-2.33 (m, 1H), 1.92-2.00 (m, 1H), 1.52 (d, J=6.6 Hz, 3H), 1.43-1.54 (m, 2H), 0.98-1.06 (m, 2H), 0.81 (br s, 2H).

### Compound 10

Major rotamer (70%)¹H NMR (500 MHz, DMSO-d₆) δ ppm 6.95-7.38 (m, 6H), 6.64 (s, 1H), 6.62 (br s, 1H), 6.48-6.54 (m, 2H), 5.57 (br s, 1H), 3.77 (br s, 1H), 3.61 (br s, 3H), 3.35-3.55 (m, 5H) 3.16-3.23 (m, 2H), 2.93-3.05 (m, 1H), 2.66-2.83 (m, 1H), 2.13-2.32 (m, 3H), 1.51 (d, J=6.9 Hz, 3H), 0.98-1.05 (m, 2H), 0.80 (br s, 2H).

Minor rotamer (30%)¹H NMR (500 MHz, DMSO-d₆) δ ppm 6.95-7.38 (m, 6H), 6.64 (s, 1H), 6.62 (br s, 1H), 6.48-6.54 (m, 2H), 4.88 (br s, 1H), 4.53 (br s, 1H), 3.61 (br s, 3H), 3.35-3.55 (m, 5H), 3.16-3.23 (m, 2H), 2.93-3.05 (m, 1H), 2.66-2.83 (m, 1H), 2.13-2.32 (m, 3H), 1.51 (d, J=6.9 Hz, 3H), 0.98-1.05 (m, 2H), 0.80 (br s, 2H).

### Compound 11

Major rotamer (65%)¹H NMR (500 MHz, DMSO-d₆) δ ppm 12.45 (br s, 1H), 7.66 (t, J=7.9 Hz, 1H), 7.35 (br d, J=11.7 Hz, 1H), 7.32 (br d, J=7.6 Hz, 1H), 7.29 (br d, J=8.2 Hz, 1H), 7.05-7.25 (m, 4H), 5.59 (q, J=6.8 Hz, 1H), 3.87 (br dd, J=13.2, 4.1 Hz, 1H), 3.71 (s, 3H), 3.24-3.45 (m, 1H), 3.11-3.20 (m, 1H), 2.73 (br d, J=15.8 Hz, 1H), 2.55-2.60 (m, 2H), 2.01 (dt, J=8.4, 4.5 Hz, 1H), 1.53 (br d, J=6.9 Hz, 3H), 1.47-1.53 (m, 2H), 1.22-1.30 (m, 2H), 1.13-1.22 (m, 2H).

Minor rotamer (35%)¹H NMR (500 MHz, DMSO-d₆) δ ppm 12.45 (br s, 1H), 7.66 (t, J=7.9 Hz, 1H), 7.35 (br d, J=11.7 Hz, 1H), 7.29 (br d, J=8.2 Hz, 1H), 7.05-7.25 (m, 5H), 5.05 (q, J=6.6 Hz, 1H), 4.57 (br dd, J=13.1, 4.9 Hz, 1H), 3.71 (s, 3H), 3.24-3.45 (m, 1H), 2.89-2.98 (m, 1H), 2.82-2.89 (m, 1H), 2.55-2.60 (m, 2H), 2.01 (dt, J=8.4, 4.5 Hz, 1H), 1.65 (d, J=6.6 Hz, 3H), 1.47-1.53 (m, 2H), 1.22-1.30 (m, 2H), 1.13-1.22 (m, 2H).

### Compound 12

Major rotamer (60%)¹H NMR (400 MHz, DMSO-d₆) δ ppm 12.57 (br s, 1H), 7.54 (t, J=8.8 Hz, 1H), 7.32 (d, J=7.6 Hz, 1H), 7.04-7.25 (m, 4H), 6.52-6.62 (m, 2H), 5.58 (q, J=7.1 Hz, 1H), 3.89 (br dd, J=13.6, 3.5 Hz, 1H), 3.71 (s, 3H), 3.48-3.62 (m, 3H), 3.34-3.46 (m, 3H), 3.21-3.30 (m, 1H), 3.10-3.21 (m, 1H), 2.73 (br d, J=16.2 Hz, 1H), 2.14-2.31 (m, 2H), 1.53 (d, J=6.6 Hz, 3H), 1.13-1.30 (m, 4H).

Minor rotamer (40%)¹H NMR (400 MHz, DMSO-d₆) δ ppm 12.57 (br s, 1H), 7.54 (t, J=8.8 Hz, 1H), 7.04-7.25 (m, 5H), 6.52-6.62 (m, 2H), 5.06 (q, J=7.1 Hz, 1H), 4.56 (br dd, J=12.6, 3.5 Hz, 1H), 3.71 (s, 3H), 3.48-3.62 (m, 3H), 3.34-3.46 (m, 3H), 3.21-3.30 (m, 1H), 2.89-2.99 (m, 1H), 2.80-2.88 (m, 1H), 2.14-2.31 (m, 2H), 1.65 (d, J=6.6 Hz, 3H), 1.13-1.30 (m, 4H).

### Compound 13

Trans major rotamer (55%)¹H NMR (500 MHz, DMSO-d₆) δ ppm 12.50 (br s, 1H), 8.12-8.25 (m, 1H), 6.94-7.58 (m, 7H), 5.60 (q, J=6.6 Hz, 1H), 3.48-4.64 (m, 5H), 2.67-3.17 (m, 3H), 2.55-2.61 (m, 1H), 1.99-2.09 (m, 1H), 1.23-1.70 (m, 9H).

Trans minor rotamer (20%)¹H NMR (500 MHz, DMSO-d₆) δ ppm 12.50 (br s, 1H), 8.12-8.25 (m, 1H), 6.94-7.58 (m, 7H), 4.73 (q, J=6.9 Hz, 1H), 3.48-4.64 (m, 5H), 2.67-3.17 (m, 3H), 2.55-2.61 (m, 1H), 1.99-2.09 (m, 1H), 1.23-1.70 (m, 9H).

Cis major rotamer (20%)¹H NMR (500 MHz, DMSO-d₆) δ ppm 12.50 (br s, 1H), 8.12-8.25 (m, 1H), 6.94-7.58 (m, 7H), 5.69 (q, J=6.6 Hz, 1H), 3.48-4.64 (m, 5H), 2.67-3.17 (m, 3H), 2.55-2.61 (m, 1H), 1.99-2.09 (m, 1H), 1.23-1.70 (m, 9H).

Cis minor rotamer (5%)¹H NMR (500 MHz, DMSO-d₆) δ ppm 12.50 (br s, 1H), 8.12-8.25 (m, 1H), 6.94-7.58 (m, 7H), 4.83-4.91 (m, 1H), 3.48-4.64 (m, 5H), 2.67-3.17 (m, 3H), 2.55-2.61 (m, 1H), 1.99-2.09 (m, 1H), 1.23-1.70 (m, 9H).

### Compound 14

Major rotamer (70%)¹H NMR (500 MHz, DMSO-d₆) δ ppm 12.08 (br s, 1H), 7.61 (t, J=7.9 Hz, 1H), 7.47 (br s, 1H), 7.25-7.37 (m, 3H), 7.12-7.25 (m, 3H), 6.83 (br s, 1H), 5.60 (br s, 1H), 3.69 (br s, 3H), 3.41 (br s, 1H), 2.93-3.03 (m, 1H), 2.61-2.85 (m, 3H), 2.52-2.56 (m, 1H), 2.13 (q, J=7.6 Hz, 1H), 1.63 (q, J=6.0 Hz, 1H), 1.52 (br d, J=6.6 Hz, 3H), 1.40 (td, J=8.0, 5.0 Hz, 1H), 0.97-1.12 (m, 4H).

Minor rotamer (30%)¹H NMR (500 MHz, DMSO-d₆) δ ppm 12.08 (br s, 1H), 7.61 (t, J=7.9 Hz, 1H), 7.47 (br s, 1H), 7.25-7.37 (m, 3H), 7.12-7.25 (m, 3H), 6.83 (br s, 1H), 4.83 (br s, 1H), 4.56 (br s, 1H), 3.69 (br s, 3H), 2.93-3.03 (m, 1H), 2.61-2.85 (m, 3H), 2.52-2.56 (m, 1H), 2.13 (q, J=7.6 Hz, 1H), 1.63 (q, J=6.0 Hz, 1H), 1.52 (br d, J=6.6 Hz, 3H), 1.40 (td, J=8.0, 5.0 Hz, 1H), 0.97-1.12 (m, 4H).

### Compound 15

Major rotamer (70%)¹H NMR (500 MHz, DMSO-d₆) δ ppm 7.56 (t, J=7.9 Hz, 1H), 7.50 (br s, 1H), 7.46 (br s, 1H), 7.06-7.35 (m, 6H), 6.83 (br s, 1H), 6.71 (br s, 1H), 5.60 (br s, 1H), 3.69 (br s, 4H), 3.16-3.49 (m, 2H), 2.91-3.06 (m, 1H), 2.65-2.85 (m, 1H), 2.52-2.57 (m, 1H), 2.11 (q, J=7.4 Hz, 1H), 1.56 (q, J=5.6 Hz, 1H), 1.52 (br d, J=6.6 Hz, 3H), 1.27 (td, J=8.1, 4.6 Hz, 1H), 0.98-1.13 (m, 4H).

Minor rotamer (30%)¹H NMR (500 MHz, DMSO-d₆) δ ppm 7.56 (t, J=7.9 Hz, 1H), 7.50 (br s, 1H), 7.46 (br s, 1H), 7.06-7.35 (m, 6H), 6.83 (br s, 1H), 6.71 (br s, 1H), 4.85 (br s, 1H), 4.56 (br s, 1H), 3.69 (br s, 3H), 3.16-3.49 (m, 2H), 2.91-3.06 (m, 1H), 2.65-2.85 (m, 1H), 2.52-2.57 (m, 1H), 2.11 (q, J=7.4 Hz, 1H), 1.56 (q, J=5.6 Hz, 1H), 1.52 (br d, J=6.6 Hz, 3H), 1.27 (td, 4.6 Hz, 1H), 0.98-1.13 (m, 4H).

### Compound 16

Major rotamer (65%)¹H NMR (500 MHz, DMSO-d₆) δ ppm 12.55 (br s, 1H), 7.49 (t, J=8.5 Hz, 1H), 7.45 (br s, 1H), 7.31 (br s, 1H), 7.22 (br s, 1H), 7.17 (br s, 2H), 6.81 (br s, 1H), 6.50-6.61 (m, 2H), 5.59 (br s, 1H), 3.69 (br s, 4H), 3.48-3.59 (m, 2H), 3.30-3.47 (m, 3H), 3.25 (br quin, J=6.9 Hz, 1H), 2.92-3.05 (m, 1H), 2.67-2.88 (m, 1H), 2.52-2.60 (m, 1H), 2.15-2.31 (m, 2H), 1.52 (br d, J=6.6 Hz, 3H), 0.96-1.07 (m, 4H).

Minor rotamer (35%)¹H NMR (500 MHz, DMSO-d₆) δ ppm 12.55 (br s, 1H), 7.49 (t, J=8.5 Hz, 1H), 7.45 (br s, 1H), 7.17 (br s, 2H), 6.97-7.13 (m, 2H), 6.81 (br s, 1H), 6.50-6.61 (m, 2H), 4.84 (br s, 1H), 4.55 (br s, 1H), 3.69 (br s, 3H), 3.48-3.59 (m, 2H), 3.30-3.47 (m, 3H), 3.25 (br quin, J=6.9 Hz, 1H), 2.92-3.05 (m, 1H), 2.67-2.88 (m, 1H), 2.52-2.60 (m, 1H), 2.15-2.31 (m, 2H), 1.52 (br d, J=6.6 Hz, 3H), 0.96-1.07 (m, 4H).

### Compound 17

¹H NMR (500 MHz, DMSO-d₆, 77° C.) 8 ppm 7.45 (t, J=8.7 Hz, 1H), 7.35 (s, 1H), 7.21-7.32 (br s, 1H), 7.13-7.21 (m, 4H), 6.79 (s, 1H), 6.64-6.76 (br s, 1H), 6.54 (dd, J=8.7, 2.0 Hz, 1H), 6.47 (dd, J=13.7, 1.7 Hz, 1H), 5.36 (br s, 1H), 4.01 (br s, 1H), 3.66 (d, J=1.3 Hz, 3H), 3.53 (t, J=8.8 Hz, 1H), 3.41-3.47 (m, 2H), 3.31-3.41 (m, 2H), 3.12 (quin, J=7.5 Hz, 1H), 2.92-3.01 (m, 1H), 2.74 (br d, J=15.8 Hz, 1H), 2.50-2.56 (m, 1H), 2.10-2.26 (m, 2H), 1.51 (d, J=6.6 Hz, 3H), 0.99-1.08 (m, 4H).

### Compound 18

Major rotamer (70%)¹H NMR (500 MHz, DMSO-d₆) δ ppm 8.62 (s, 1H), 7.77 (dd, J=13.4, 1.7 Hz, 1H), 7.51-7.60 (m, 2H), 7.45 (br s, 1H), 7.09-7.34 (m, 4H), 6.81 (br s, 1H), 5.59 (br s, 1H), 4.99 (d, J=3.8 Hz, 1H), 4.33 (br s, 1H), 3.70 (br s, 3H), 3.45-3.54 (m, 3H), 3.41 (br s, 1H), 3.35 (br d, J=10.7 Hz, 1H), 2.94-3.04 (m, 1H), 2.67-2.80 (m, 1H), 2.51-2.57 (m, 1H), 1.91-1.98 (m, 1H), 1.80-1.87 (m, 1H), 1.52 (d, J=6.9 Hz, 3H), 0.99-1.11 (m, 4H).

Minor rotamer (30%)¹H NMR (500 MHz, DMSO-d₆) δ ppm 8.62 (s, 1H), 7.77 (dd, J=13.4, 1.7 Hz, 1H), 7.51-7.60 (m, 2H), 7.45 (br s, 1H), 7.09-7.34 (m, 4H), 6.81 (br s, 1H), 4.99 (d, J=3.8 Hz, 1H), 4.84 (br s, 1H), 4.55 (br s, 1H), 4.33 (br s, 1H), 3.70 (br s, 3H), 3.45-3.54 (m, 3H), 3.35 (br d, J=10.7 Hz, 1H), 2.94-3.04 (m, 1H), 2.67-2.80 (m, 1H), 2.51-2.57 (m, 1H), 1.91-1.98 (m, 1H), 1.80-1.87 (m, 1H), 1.52 (d, J=6.9 Hz, 3H), 0.99-1.11 (m, 4H).

### Compound 19

Major rotamer (70%)¹H NMR (400 MHz, DMSO-d₆) δ ppm 8.63 (d, J=2.1 Hz, 1H), 7.60 (s, 1H), 7.52 (t, J=8.9 Hz, 1H), 7.29 (br s, 1H), 7.11-7.25 (br s, 3H), 6.84 (br s, 1H), 6.55-6.58 (m, 1H), 6.54 (s, 1H), 5.86 (s, 2H), 5.57 (br s, 1H), 3.76 (br s, 1H), 3.35-3.49 (m, 1H), 2.90-3.03 (m, 1H), 2.70-2.81 (m, 1H), 2.39-2.47 (m, 1H), 1.50 (d, J=6.7 Hz, 3H), 0.98-1.13 (m, 4H).

Minor rotamer (30%)¹H NMR (400 MHz, DMSO-d₆) δ ppm 8.63 (d, J=2.1 Hz, 1H), 7.60 (s, 1H), 7.52 (t, J=8.9 Hz, 1H), 7.29 (br s, 1H), 7.11-7.25 (br s, 3H), 6.84 (br s, 1H), 6.55-6.58 (m, 1H), 6.54 (s, 1H), 5.86 (s, 2H), 4.80-4.99 (m, 1H), 4.44-4.64 (m, 1H), 3.35-3.49 (m, 1H), 2.90-3.03 (m, 1H), 2.70-2.81 (m, 1H), 2.39-2.47 (m, 1H), 1.50 (d, J=6.7 Hz, 3H), 0.98-1.13 (m, 4H).

### Compound 20

Major rotamer (70%)¹H NMR (400 MHz, DMSO-d₆) δ ppm 8.79 (d, J=2.2 Hz, 1H), 8.64 (s, 1H), 7.78-7.90 (m, 2H), 7.64 (br s, 1H), 7.54 (dd, J=9.0, 1.4 Hz, 1H), 7.02-7.40 (m, 4H), 6.88 (br s, 1H), 5.58 (br s, 1H), 5.02 (d, J=3.3 Hz, 1H), 4.33 (br s, 1H), 3.76 (br s, 1H), 3.43-3.55 (m, 3H), 3.35 (br s, 1H), 2.91-3.03 (m, 1H), 2.70-2.84 (m, 1H), 2.40-2.47 (m, 1H), 1.90-2.01 (m, 1H), 1.78-1.88 (m, 1H), 1.51 (d, J=6.6 Hz, 3H), 1.00-1.14 (m, 4H).

Minor rotamer (30%)¹H NMR (400 MHz, DMSO-d₆) δ ppm 8.79 (d, J=2.2 Hz, 1H), 8.64 (s, 1H), 7.78-7.90 (m, 2H), 7.64 (br s, 1H), 7.54 (dd, J=9.0, 1.4 Hz, 1H), 7.02-7.25 (m, 4H), 6.88 (br s, 1H), 5.02 (d, J=3.3 Hz, 1H), 4.82-4.95 (br s, 1H), 4.46-4.63 (br s, 1H), 4.33 (br s, 1H), 3.43-3.55 (m, 3H), 3.35 (br s, 1H), 2.91-3.03 (m, 1H), 2.70-2.84 (m, 1H), 2.40-2.47 (m, 1H), 1.90-2.01 (m, 1H), 1.78-1.88 (m, 1H), 1.51 (d, J=6.6 Hz, 3H), 1.00-1.14 (m, 4H).

### Compound 21

Major rotamer (75%)¹H NMR (500 MHz, DMSO-d₆, 38° C.) 8 ppm 12.45 (br s, 1H), 7.98 (t, J=8.7 Hz, 1H), 7.51 (br s, 1H), 7.05-7.36 (m, 4H), 6.95 (s, 1H), 6.59 (br d, J=8.8 Hz, 1H), 6.52 (br d, J=14.2 Hz, 1H), 5.58 (br s, 1H), 3.63-3.76 (m, 1H), 3.50-3.62 (m, 2H), 3.31-3.49 (m, 3H), 2.89-3.05 (m, 1H), 2.68-2.85 (m, 1H), 2.38-2.47 (m, 2H), 2.15-2.32 (m, 2H), 1.50 (br d, J=6.3 Hz, 3H), 1.04-1.15 (m, 4H).

Minor rotamer (25%)¹H NMR (500 MHz, DMSO-d₆, 38° C.) 8 ppm 12.45 (br s, 1H), 7.98 (t, J=8.7 Hz, 1H), 7.51 (br s, 1H), 7.05-7.36 (m, 4H), 6.95 (s, 1H), 6.59 (br d, J=8.8 Hz, 1H), 6.52 (br d, J=14.2 Hz, 1H), 4.80 (br s, 1H), 4.53 (br, 1H), 3.50-3.62 (m, 2H), 3.31-3.49 (m, 3H), 2.89-3.05 (m, 1H), 2.68-2.85 (m, 1H), 2.38-2.47 (m, 2H), 2.15-2.32 (m, 2H), 1.50 (br d, J=6.3 Hz, 3H), 1.04-1.15 (m, 4H).

### Compound 22

Major rotamer (70%)¹H NMR (500 MHz, DMSO-d₆, 35° C.) 8 ppm 7.98 (br t, J=8.7 Hz, 1H), 7.52 (br s, 1H), 7.47 (br s, 1H), 7.29 (br s, 1H), 7.10-7.23 (m, 3H), 6.96 (br s, 2H), 6.57 (br d, J=8.8 Hz, 1H), 6.50 (br d, J=14.5 Hz, 1H), 5.58 (br s, 1H), 3.67 (br s, 1H), 3.55 (br t, J=9.0 Hz, 1H), 3.32-3.51 (m, 4H), 3.11 (quin, J=7.3 Hz, 1H), 2.91-3.03 (m, 1H), 2.74 (br s, 1H), 2.39-2.48 (m, 1H), 2.18-2.27 (m, 1H), 2.07-2.18 (m, 1H), 1.51 (br d, J=6.0 Hz, 3H), 1.04-1.15 (m, 4H).

Minor rotamer (30%)¹H NMR (500 MHz, DMSO-d₆, 35° C.) 8 ppm 7.98 (br t, J=8.7 Hz, 1H), 7.52 (br s, 1H), 7.47 (br s, 1H), 7.29 (br s, 1H), 7.10-7.23 (m, 3H), 6.96 (br s, 2H), 6.57 (br d, J=8.8 Hz, 1H), 6.50 (br d, J=14.5 Hz, 1H), 4.80 (br s, 1H), 4.55 (br s, 1H), 3.55 (br t, J=9.0 Hz, 1H), 3.32-3.51 (m, 4H), 3.11 (quin, J=7.3 Hz, 1H), 2.91-3.03 (m, 1H), 2.74 (br s, 1H), 2.39-2.48 (m, 1H), 2.18-2.27 (m, 1H), 2.07-2.18 (m, 1H), 1.51 (br d, J=6.0 Hz, 3H), 1.04-1.15 (m, 4H).

### Compound 23

¹H NMR (500 MHz, DMSO-d₆) δ ppm 8.76 (s, 1H), 8.08 (t, J=8.5 Hz, 1H), 7.81 (dd, J=14.5, 1.9 Hz, 1H), 7.49-7.66 (m, 2H), 6.93-7.26 (m, 5H), 5.34-5.70 (m, 0.7H), 4.99 (d, J=3.5 Hz, 1H), 4.68-4.89 (m, 0.3H), 4.45-4.63 (m, 0.3H), 4.32 (br s, 1H), 3.32-3.77 (m, 5.7H), 2.91-3.02 (m, 1H), 2.65-2.89 (m, 1H), 2.42-2.47 (m, 1H), 1.89-2.03 (m, 1H), 1.79-1.90 (m, 1H), 1.51 (br d, J=5.7 Hz, 3H), 1.12 (br d, J=5.0 Hz, 4H).

### Compound 24

¹H NMR (500 MHz, DMSO-d₆) δ ppm 8.73 (s, 1H), 8.27 (t, J=8.83 Hz, 1H), 7.98 (br s, 1H), 7.84 (dd, J=15.1, 1.9 Hz, 1H), 7.56 (dd, J=8.8, 1.9 Hz, 1H), 6.92-7.26 (m, 5H), 5.51-5.65 (m, 0.70H), 4.98 (d, J=3.5 Hz, 1H), 4.69-4.84 (m, 0.3H), 4.50-4.67 (m, 0.3H), 4.26-4.38 (m, 1H), 3.32-3.72 (m, 5.7H), 2.92-3.05 (m, 1H), 2.88 (br qt, J=5.0 Hz, 1H), 2.66-2.80 (m, 1H) 1.90-2.01 (m, 1H), 1.78-1.89 (m, 1H), 1.40-1.65 (m, 3H), 1.11-1.19 (m, 2H), 0.96-1.08 (m, 2H).

### Compound 25

Major rotamer (65%)¹H NMR (400 MHz, DMSO-d₆) δ ppm 8.80 (s, 1H) 7.67-7.94 (m, 4H) 7.43-7.63 (m, 3H) 7.04-7.39 (m, 5H) 6.45-6.69 (m, 2H) 5.63-5.73 (m, 1H) 5.24 (d, J=3.2 Hz, 2H) 4.10 (br s, 2H) 3.77-3.94 (m, 1H) 3.44-3.63 (m, 3H) 3.20 (br d, J=10.5 Hz, 2H) 2.72-3.12 (m, 2H) 1.57 (t, J=6.7 Hz, 3H)

Minor rotamer (35%)¹H NMR (400 MHz, DMSO-d₆) δ ppm 8.80 (s, 1H) 7.67-7.94 (m, 4H) 7.43-7.63 (m, 3H) 7.04-7.39 (m, 5H) 6.45-6.69 (m, 2H) 5.24 (d, J=3.2 Hz, 2H) 4.90-5.11 (m, 1H) 5.55-5.73 (m, 1H) 4.10 (br s, 2H) 3.44-3.63 (m, 3H) 3.20 (br d, J=10.5 Hz, 2H) 2.72-3.12 (m, 2H) 1.57 (t, J=6.7 Hz, 3H)

LC-MS Data

The High Performance Liquid Chromatography (HPLC) measurement was performed using a LC pump, a diode-array (DAD) or a UV detector and a column as specified in the respective methods. If necessary, additional detectors were included (see table of methods below). Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

Table: LCMS Method codes (Flow expressed in mL/min; column temperature (T) in ° C.; Run time in minutes).

| Method | | | | | Flow | |
|---|---|---|---|---|---|---|
| code | Instrument | Column | Mobile phase | gradient | Column T | Run time |
| 1 | Waters: | Waters: | A: 95% | 84.2% A for | 0.343 | 6.2 |
| | Acquity | BEH C18 | CH₃COONH₄ | 0.49 min, to 10.5% | 40 | |
| | UPLC ® - DAD | (1.7 μm, | 7 mM/5% | A in 2.18 min, held | | |
| | and Quattro | 2.1 × | CH₃CN, | for 1.94 min, back | | |
| | Micro ™ | 100 mm) | B: CH₃CN | to 84.2% A in 0.73 | | |
| | | | | min, held for 0.73 | | |
| | | | | min. | | |
| 2 | Waters: | Waters: | A: 95% | 84.2% A for | 0.343 | 6.07 |
| | Acquity | BEH C18 | CH₃COONH₄ | 0.49 min, to 10.5% | 40 | |
| | UPLC ® H- | (1.7 μm, | 7 mM/5% | A in | | |
| | Class - DAD | 2.1 × | CH₃CN, | 2.18 min, held | | |
| | and SQD 2 | 100 mm) | B: CH₃CN | for 1.94 min, back | | |
| | | | | to 84.2% A in | | |
| | | | | 0.73 min, held for | | |
| | | | | 0.73 min. | | |
| | | | | | 40 | |
| 3 | | | | | | |
| | Waters: | Waters | A: 95% | From 95% A to 5% | 0.5 | 3.3 |
| | Acquity | BEH ®C18 | CH₃COONH₄ | A in 1 min, held for | 40 | |
| | UPLC ® H- | (1.7 μm, | 7 mM/5% | 1.6 min, back to | | |
| | Class - DAD | 2.1 × | CH₃CN, | 95% A in 0.2 min, | | |
| | and SQD 2 | 50 mm) | B: CH₃CN | held for 0.5 min. | | |

| Co. No. | Rt | MW (theor) | BPM1 [M + H]+ | LC/GC/MS Method |
|---|---|---|---|---|
| 1 | 2.86 | 543.2 | 544.5 | 1 |
| 2 | 2.9 | 528.2 | 529.3 | 1 |
| 3 | 3.17 | 514.2 | 515.3 | 1 |
| 4 | 3.11 | 543.2 | 544.3 | 1 |
| 5 | 2.95 | 544.2 | 545.4 | 1 |
| 6 | 2.87 | 542.2 | 543.5 | 1 |
| 7 | 2.7 | 554.2 | 555.3 | 1 |
| 8 | 2.66 | 508.22 | 509.1 | 1 |
| 9 | 2.73 | 522.2 | 523.4 | 1 |
| 10 | 2.79 | 551.2 | 552.5 | 1 |
| 11 | 2.38 | 524.2 | 525.3 | 1 |
| 12 | 2.43 | 553.2 | 554.4 | 1 |
| 13 | 2.23 | 524.2 | 525.4 | 1 |
| 14 | 2.39 | 523.2 | 524.3 | 1 |
| 15 | 2.77 | 522.2 | 523.3 | 1 |
| 16 | 1.21 | 552.2 | 553.4 | 3 |
| 17 | 2.91 | 551.3 | 552.4 | 1 |
| 18 | 2.66 | 567.3 | 568.4 | 1 |
| 19 | 3.11 | 440.2 | 441.2 | 1 |
| 20 | 2.84 | 553.2 | 554.4 | 1 |
| 21 | 2.57 | 539.2 | 540.3 | 1 |
| 22 | 3.1 | 538.2 | 539.4 | 1 |
| 23 | 2.82 | 554.2 | 555.5 | 2 |
| 24 | 3.08 | 570.2 | 571.5 | 1 |
| 25 | 3.06 | 562.2 | 563.4 | 1 |

Optical Rotation

The optical rotation was measured using a polarimeter with light at the wavelength of the D-line of sodium (589 nm) at a temperature of 20° C. in DMF as solvent. Specific optical rotation of compounds (1), (3) and (10) was measured at 436 nm in DMF at 20° C. as solvent.

| Co. No. | OR |
|---|---|
| 1 | +67.96° (436 nm, c 0.309 w/v %, DMF, 20° C.) |
| 2 | +50.69° (589 nm, c 0.29 w/v %, DMF, 20° C.) |
| 3 | +5.68° (436 nm, c 0.25 w/v %, DMF, 20° C.) |
| 4 | +5.56° (589 nm, c 0.27 w/v %, DMF, 20° C.) |
| 5 | +6.67° (589 nm, c 0.27 w/v %, DMF, 20° C.) |
| 6 | −21.29° (589 nm, c 0.31 w/v %, DMF, 20° C.) |
| 7 | −25.33° (589 nm, c 0.3 w/v %, DMF, 20° C.) |
| 8 | +93.13° (589 nm, c 0.2212 w/v %, DMF, 20° C.) |
| 9 | +163° (589 nm, c 0.3 w/v %, DMF, 20° C.) |
| 10 | +14.62° (436 nm, c 0.26 w/v %, DMF, 20° C.) |
| 11 | +17.86° (589 nm, c 0.28 w/v %, DMF, 20° C.) |
| 12 | +57.69° (589 nm, c 0.26 w/v %, DMF, 20° C.) |
| 16 | +7.93° (589 nm, c 0.2522 w/v %, DMF, 20° C.) |
| 17 | −28.37° (589 nm, c 0.208 w/v %, DMF, 20° C.) |
| 18 | −27.67° (589 nm, c 0.3 w/v %, DMF, 20° C.) |
| 20 | −24.69° (589 nm, c 0.32 w/v %, DMF, 20° C.) |
| 21 | −23.4° (589 nm, c 0.282 w/v %, DMF, 20° C.) |
| 22 | −24.03° (589 nm, c 0.258 w/v %, DMF, 20° C.) |
| 23 | −26.29° (589 nm, c 0.232 w/v %, DMF, 20° C.) |
| 24 | −27.85° (589 nm, c 0.219 w/v %, DMF, 20° C.) |

### E. Pharmacological Examples

E.1 Antiviral Activity

Black 384-well clear-bottom microtiter plates (Corning, Amsterdam, The Netherlands) were filled via acoustic drop ejection using the echo liquid handler (Labcyte, Sunnyvale, California). 200 nL of compound stock solutions (100% DMSO) were transferred to the assay plates. 9 serial 4-fold dilutions of compound were made, creating per quadrant the same compound concentration. The assay was initiated by adding 10 μL of culture medium to each well (RPMI medium without phenol red, 10% FBS-heat inactivated, 0.04% gentamycin (50 mg/mL). All addition steps are done by using a multidrop dispenser (Thermo Scientific, Erembodegem, Belgium). Next, rgRSV224 virus (MOI=1) diluted in culture medium was added to the plates. rgRSV224 virus is an engineered virus that includes an additional GFP gene (Hallak LK, Spillmann D, Collins PL, Peeples ME. Glycosaminoglycan sulfation requirements for respiratory syncytial virus infection; Journal of virology (2000), 74(22), 10508-13) and was in-licensed from the NIH (Bethesda, MD, USA). Finally, 20 μL of a HeLa cell suspension (3,000 cells/well) were plated. Medium, virus- and mock-infected controls were included in each test. The wells contain 0.05% DMSO per volume. Cells were incubated at 37° C. in a 5% CO₂ atmosphere. Three days post-virus exposure, viral replication was quantified by measuring GFP expression in the cells by an in house developed MSM laser microscope (Tibotec, Beerse, Belgium). The EC₅₀ was defined as the 50% inhibitory concentration for GFP expression. In parallel, compounds were incubated for three days in a set of white 384-well microtiter plates (Corning) and the cytotoxicity of compounds in HeLa cells was determined by measuring the ATP content of the cells using the ATPlite kit (Perkin Elmer, Zaventem, Belgium) according to the manufacturer's instructions. The CC₅₀ was defined as the 50% concentration for cytotoxicity.

TABLE
antiviral data (averaged data of several repeat experiments)

| | RSV HELA | TOX HELA |
|---|---|---|
| Co. No. | EC₅₀ (μM) | CC₅₀ (μM) |
| 1 | 0.01 | >25 |
| 2 | 0.02 | 26.01 |
| 3 | 0.25 | 24.15 |
| 4 | 0.14 | 14.35 |
| 5 | 0.73 | 33.26 |
| 6 | 0.07 | >100 |
| 7 | 0.05 | 47.61 |
| 8 | 2.04 | 20.94 |
| 9 | 2.31 | 31.43 |
| 10 | 2.64 | 21.07 |
| 11 | 0.52 | 76.78 |
| 12 | 0.43 | 63.02 |
| 13 | 22.36 | >100 |
| 14 | 9.05 | >100 |
| 15 | 0.69 | 50.35 |
| 16 | 0.61 | 65.32 |
| 17 | 0.17 | 39.56 |
| 18 | 0.14 | 51.65 |
| 19 | 0.16 | 55.25 |
| 20 | 0.06 | 54.35 |
| 21 | 0.01 | 51.46 |
| 22 | 0.01 | 54.77 |
| 23 | 0.05 | 47.75 |
| 24 | 0.17 | 54.03 |
| 25 | 0.47 | 19.40 |

### F. Prophetic Composition Examples

“Active ingredient” as used throughout these examples relates to a final compound of Formula (I), the pharmaceutically acceptable salts thereof, the solvates and the stereochemically isomeric forms and the tautomers thereof.

Typical examples of recipes for the formulation of the invention are as follows:

F.1. Tablets

| | |
|---|---|
| Active ingredient | 5 to 50 mg |
| Di calcium phosphate | 20 mg |
| Lactose | 30 mg |
| Talcum | 10 mg |
| Magnesium stearate | 5 mg |
| Potato starch | ad 200 mg |

In this Example, active ingredient can be replaced with the same amount of any of the compounds according to the present invention, in particular by the same amount of any of the exemplified compounds.

F.2. Suspension

An aqueous suspension is prepared for oral administration so that each 1 milliliter contains 1 to 5 mg of one of the active compounds, 50 mg of sodium carboxymethyl cellulose, 1 mg of sodium benzoate, 500 mg of sorbitol and water ad 1 ml.

F.3. Injectable

A parenteral composition is prepared by stirring 1.5% by weight of active ingredient of the invention in 10% by volume propylene glycol in water.

F.4. Ointment

| | |
|---|---|
| Active ingredient | 5 to 1000 mg |
| Stearyl alcohol | 3 g |
| Lanoline | 5 g |
| White petroleum | 15 g |
| Water | ad 100 g |

In this Example, active ingredient can be replaced with the same amount of any of the compounds according to the present invention, in particular by the same amount of any of the exemplified compounds.

1. A compound of formula (I) 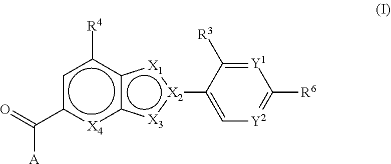
or a stereochemically isomeric form thereof, wherein
A is 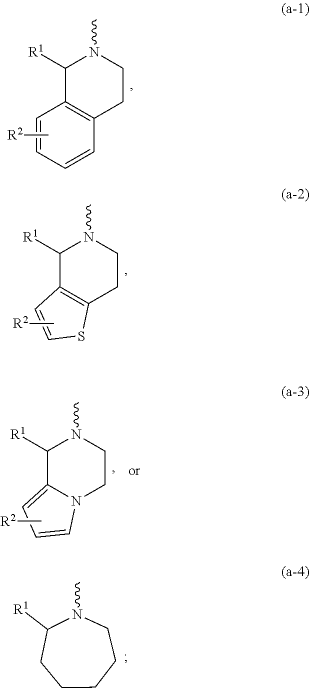
X₁, X₂, X₃, and X₄ are each independently selected from C, CH, N, NR⁵, O or S with the proviso that none of X₁, X₂, X₃, and X₄ are all C or CH;
Y¹ and Y² are each independently selected from CH, CF and N;
R¹ is CH₃ or CH₂CH₃;
R² is hydrogen, halo or C₁₋₄alkyl;
R³ is halo;
R⁴ is C₁₋₆alkyl; C₃₋₆cycloalkyl; di(C₁₋₄alkyl)amino; pyrrolidinyl; phenyl; pyridine; or
phenyl or pyridine substituted with 1, 2 or 3 substituents each individually selected from halo, hydroxy, cyano, C₁₋₄alkyl, polyhaloC₁₋₄alkyl, and C₁₋₄alkyloxy;
R⁵ is hydrogen or C₁₋₄alkyl;
R⁶ is NH₂ or a substituent selected from substituent (a) or (b); wherein
(a) is —NR⁷—(CO)-Heterocycle wherein said Heterocycle is substituted with one, two or three substituents each independently selected from halo, hydroxy, C₁₋₄alkyl of C₁₋₄alkyloxy; or
(b) is C₃₋₆cycloalkyl or Heterocycle, wherein said C₃₋₆cycloalkyl and Heterocycle is substituted with one, two or three substituents each independently selected from C₁₋₆alkyl;
C₁₋₆alkyl substituted with one, two or three substituents each independently selected from halo, hydroxy, hydroxycarbonyl, and aminocarbonyl;
hydroxy;
halo;
—(CO)—OH;
—(CO)—NR¹⁰R¹¹;
—(CO)—NR⁸—SO₂—R⁹;
—NR⁸R⁹;
—NR⁸—(CO)—C₁₋₄alkyl;
—NR⁸—(CO)—C₃₋₆cycloalkyl;
—NR⁸—SO₂—R⁹;
—SO₂—NR¹⁰R¹¹; or
—SO₂—NR⁸—(CO)—R⁹;
wherein
R⁷ is hydrogen or C₁₋₄alkyl;
each R⁸ is independently selected from hydrogen, C₁₋₄alkyl, or hydroxyC₁₋₄alkyl;
R⁹ is C₁₋₄alkyl, polyhaloC₁₋₄alkyl, or C₃₋₆cycloalkyl;
R¹⁰ and R¹¹ are each independently selected from hydrogen; C₁₋₄alkyl;
polyhaloC₁₋₄alkyl; C₃₋₆cycloalkyl; C₃₋₆cycloalkyl substituted with C₁₋₄alkyl; or C₁₋₄alkyl substituted with hydroxy or cyano;
Heterocycle is azetidinyl, pyrrolidinyl, piperidinyl, or homopiperidinyl;
or a pharmaceutically acceptable acid addition salt thereof.

2. A compound as claimed in claim 1, wherein X₁, X₂, X₃, and X₄ are selected from

| X₁ | X₂ | X₃ | X₄ | |
|---|---|---|---|---|
| CH | C | NR⁵ | CH | (b-1) |
| N | C | NR⁵ | CH | (b-2) |
| N | C | NR⁵ | N | (b-3) |
| NR⁵ | C | N | N | (b-4) |
| CH | N | N | CH | (b-5) |
| N | N | CH | CH | (b-6) |
| N | C | O | CH | (b-7) |
| N | N | CH | N | (b-8) |
| N | C | S | CH | (b-9) |
| O | C | CH | N | (b-10) |
| N | C | O | N | (b-11) |
| O | C | CH | CH | (b-12) |
| CH | C | S | N | (b-13) |
| S | C | N | N | (b-14). |

3. The compound as claimed in claim 1, wherein
radical A is (a-1);
Y¹ and Y² are each independently selected from CH;
R¹ is CH₃;
R² is hydrogen;
R³ is halo;
R⁴ is C₁₋₆alkyl, C₃₋₆cycloalkyl, or phenyl;
R⁵ is hydrogen or C₁₋₄alkyl;
R⁶ is NH₂ or a substituent selected from substituent (a) or (b); wherein
(a) is —NR⁷—(CO)-Heterocycle wherein said Heterocycle is substituted with hydroxy and R⁷ is hydrogen; or
(b) is C₃₋₆cycloalkyl or Heterocycle, wherein said C₃₋₆cycloalkyl and Heterocycle is substituted with one or two substituents each independently selected from hydroxy, —(CO)—OH or —(CO)—NR¹⁰R¹¹, wherein R¹⁰ and R¹¹ are each hydrogen; and
Heterocycle is pyrrolidinyl.

4. The compound as claimed in claim 2, wherein
radical A is (a-1);
Y¹ and Y² are each independently selected from CH;
R¹ is CH₃;
R² is hydrogen;
R³ is halo;
R⁴ is C₁₋₆alkyl, C₃₋₆cycloalkyl, or phenyl;
R⁵ is hydrogen or C₁₋₄alkyl;
R⁶ is NH₂ or a substituent selected from substituent (a) or (b); wherein
(a) is —NR⁷—(CO)-Heterocycle wherein said Heterocycle is substituted with hydroxy; or
(b) is C₃₋₆cycloalkyl or Heterocycle, wherein said C₃₋₆cycloalkyl and Heterocycle is substituted with one or two substituents each independently selected from hydroxy, —(CO)—OH or —(CO)—NR¹⁰R¹¹, wherein R¹⁰ and R¹¹ are each hydrogen; and
Heterocycle is pyrrolidinyl.

5. A pharmaceutical composition comprising a pharmaceutically acceptable carrier and a therapeutically active amount of a compound as claimed in claim 1.

6. The pharmaceutical composition according to claim 5, which further comprises another antiviral agent.

7. The pharmaceutical composition according to claim 6, wherein the another antiviral agent is a RSV inhibiting compound.

8. A process for preparing a pharmaceutical composition according to claim 5, comprising mixing the compound of formula (I) with a pharmaceutically acceptable carrier.

9. (canceled)

10. A method of treating a respiratory syncytial virus infection in a subject in need thereof, comprising administering to the subject the compound according to claim 1.

11. A method of treating a respiratory syncytial virus infection in a subject in need thereof, comprising administering to the subject the pharmaceutical composition according to claim 5.
